(19) United States
(12) Patent Application Publication
(10) Pub. No.: US 2023/0136595 A1
(43) Pub. Date: May 4, 2023

(54) METHOD FOR THE PRODUCTION AND PURIFICATION OF MULTIVALENT IMMUNOGLOBULIN SINGLE VARIABLE DOMAINS

(71) Applicant: Ablynx NV, Zwijnaarde (BE)

(72) Inventors: Florian Madura, Zwijnaarde (BE); Sonia Letestu, Zwijnaarde (BE); Ann Brigé, Ertvelde (BE); Tom Merchiers, Oudenaarde (BE); Ellen Van Horen, Zwijnaarde (BE); Chakib Borsali, Zwijnaarde (BE)

(73) Assignee: Ablynx NV, Zwijnaarde (BE)

(21) Appl. No.: 17/912,953

(22) PCT Filed: Mar. 30, 2021
(86) PCT No.: PCT/EP2021/058302

§ 371 Date: Sep. 20, 2022

(30) Foreign Application Priority Data
Mar. 30, 2020 (EP) 20166803.5

(51) Int. Cl. C07K 16/06; C07K 16/28; C07K 16/24; C07K 16/18; G01N 30/88; G01N 30/86; G01N 30/96

(52) U.S. Cl. CPC C07K 16/065; C07K 16/2875; C07K 16/241; C07K 16/18; C07K 16/248; G01N 30/88; G01N 30/8624; G01N 30/96; C07K 2317/569; C07K 2317/14; C07K 2317/31; C07K 2317/35; G01N 2030/027

(57) ABSTRACT

The present disclosure relates to an improved method for the manufacture of polypeptides comprising at least three or at least four immunoglobulin single variable domains (ISVDs). More specifically, an improved method is provided of producing, purifying and isolating polypeptides comprising at least three or at least four ISVDs in which an undesired product-related conformational variant is reduced or absent.

### 1 FIELD OF THE TECHNOLOGY

The present application relates to the field of the production and purification of immunoglobulin single variable domains (ISVDs).

The application provides a method for the manufacturing of polypeptides comprising at least three or at least four ISVDs. More specifically, an improved method is provided for producing, purifying and isolating polypeptides comprising at least three or at least four ISVDs in which a product-related conformational variant is reduced or absent. The polypeptides comprising at least three or at least four ISVDs produced/purified according to the method are superior in terms of product homogeneity because the product-related conformational variant is reduced or absent. This is beneficial e.g. in the context of a therapeutic application of the polypeptide comprising at least three or at least four ISVDs. The method thus provides for the manufacturing of homogeneous polypeptides comprising at least three or at least four ISVDs, wherein increased homogeneity and/or potencies are obtained. Therefore, the present application also describes improved compositions comprising polypeptides comprising at least three or at least four ISVDs for therapeutic use, obtainable by the present methods.

### 2 BACKGROUND ART

For therapeutic applications, immunoglobulins must be of very high product quality. This requires, amongst others, homogeneity in structural terms. Moreover, the production costs are strongly influenced by difficulties encountered during the production process. Low yields or lack of homogeneity will impact the economics of the production process, and hence, the costs for the therapeutic, overall. For example, difficulties to separate structural variants of a desired protein from the desired protein will necessitate complex and costly purification strategies.

Amongst other requirements, therapeutic proteins must be fully functional. Protein function depends, amongst other factors, on the chemical and physical stability of the protein during fermentation, purification and storage. Chemical instability may be caused, amongst others, by deamidation, isomerization, racemization, hydrolysis, oxidation, pyroglutamate formation, carbamylation, beta elimination and/or disulfide exchange. Physical instability may be caused by antibody denaturation, aggregation, precipitation or adsorption. Among those, aggregation, deamidation and oxidation are known to be the most common causes of the antibody degradation (Cleland et al., 1993, Critical Reviews in Therapeutic Drug Carrier Systems 10: 307-377).

The limitation of obtaining adequate yields of functional product has been reported for conventional immunoglobulins and their fragments across a broad range of expression systems, including, amongst others, in vitro translation, E. coli, Saccharomyces cerevisiae, Chinese hamster ovary cells, baculovirus systems in insect cells and Pichia pastoris (Ryabova et al., Nature Biotechnology 15: 79, 1997; Humphreys et al., FEES Letters 380: 194, 1996; Shusta et al., Nature Biotech. 16: 773, 1998; Hsu et al., Protein Expr.& Purif. 7: 281, 1996; Mohan et al., Biotechnol. & Bioeng. 98: 611, 2007; Xu et al., Metabol. Engineer. 7: 269, 2005; Merk et al., J. Biochem. 125: 328, 1999; Whiteley et al., J. Biol. Chem. 272: 22556, 1997; Gasser et al., Biotechnol. Bioeng. 94: 353, 2006; Demarest and Glaser, Curr. Opin. Drug Discov. Devel. 11(5): 675-87, 2008; Honegger, Handb. Exp. Pharmacol. 181: 47-68, 2008; Wang et al., J. Pharm. Sci. 96(1): 1-26, 2007).

In contrast to these difficulties observed, immunoglobulin single variable domains (ISVDs) can be readily expressed in a fully functional form in different host cells, like prokaryotic organisms such as E. coli, lower eukaryotes such as P. pastoris, or higher eukaryotes such as CHO cells at a sufficient rate and level. Biopharmaceutical production of ISVDs in higher eukaryotes such as mammalian cells (e.g. CHO cells) as for example described in WO 2010/056550, often requires virus clearance/inactivation in the downstream purification process by low pH treatment. In lower eukaryotes such as yeast the problem of virus inactivation does not exit. Immunoglobulin single variable domains are characterized by formation of the antigen binding site by a single variable domain, which does not require interaction with a further domain (e.g. in the form of VH/VL interaction) for antigen recognition. Production of NANOBODY® ISVDs, as one specific example of an immunoglobulin single variable domain, has been extensively described e.g. in WO 94/25591.

Despite these supposed advantages, problems in producing structurally homogeneous ISVD product have been reported. For instance, in WO 2010/125187 it was shown that the production of ISVDs may be accompanied by product-related variants lacking at least one disulfide bridge. Moreover, WO2012/05600 describes the presence of a structural variant of the produced ISVD that comprises at least one carbamylated amino acid residue.

However, further specific problems for obtaining structurally homogeneous and functional ISVD products comprising at least three or at least four ISVDs have not been reported.

### 3 SUMMARY

A product-related conformational variant was observed during the production process of a multivalent polypeptide product comprising at least three or at least four ISVDs. The product-related conformational variant was observed upon production of the multivalent polypeptide product comprising at least three or at least four ISVDs in a host, in particular in a host that is a lower eukaryotic host such as yeast. It could be revealed that the conformational variant of the multivalent polypeptide product comprising at least three or at least four ISVDs results from expression of the polypeptide in a host, in particular in a host that is a lower eukaryotic host such as yeast. The present inventors could identify the product-related conformation variant by specific analytical chromatographic techniques such as analytical SE-HPLC and/or analytical IEX-HPLC as provided herein. The present technology relates to methods of producing, purifying, and isolating multivalent polypeptides comprising at least three or at least four ISVDs, characterized by the reduction or absence of the product-related conformational variant.

The present application provides a method of isolating or purifying a polypeptide that comprises or consists of at least three or at least four immunoglobulin single variable domains (ISVDs) from a composition comprising the polypeptide and a conformational variant thereof, wherein the method comprises:
a) applying conditions that convert the conformational variant into the (desired) polypeptide;
b) removing the conformational variant; or
c) a combination of (a) and (b).

The polypeptide to be isolated/purified by the methods provided in the present application is obtainable by expression in a host. The polypeptide to be isolated/purified by the methods provided in the present application is obtainable by expression in a host that is not a CHO cell. The polypeptide to be isolated/purified by the methods provided in the present application is obtainable by expression in a lower eukaryotic host such as yeast. The conformational variant results from expression of the polypeptide in a host, in particular in a host that is a lower eukaryotic host such as yeast. Without being limiting, the yeast can be Pichia (Komagataella), Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis. In one aspect, the polypeptide to be isolated/purified by the methods provided in the present application is obtainable by expression in Pichia, in particular in Pichia pastoris.

In one embodiment, the percentage (%) of conformational variant in the compositions is reduced to 5% or less. In another embodiment, the percentage (%) of conformational variant in the compositions is reduced to 4% or less, 3% or less, 2% or less, 1% or less, such as 0.5%, 0.1% or even 0% conformational variant.

The conformational variant to be converted and/or to be removed by the methods described herein is characterized by a more compact form. The conformational variant that is to be converted and/or to be removed by the methods described herein is also characterized by a decreased hydrodynamic volume. The compact form of the conformational variant can be due to a decreased hydrodynamic volume. The conformational variant can also be characterized by an altered surface charge and/or surface hydrophobicity. The conformational variant can thus be characterized by a decreased hydrodynamic volume, an altered surface charge, and/or altered surface hydrophobicity. Without being bound by any hypothesis, the conformational variant to be converted and/or to be removed by the methods described herein might be characterized by weak intra-molecular interactions between ISVD building blocks present in the polypeptide, resulting in a decreased hydrodynamic volume, an altered surface charge, and/or altered surface hydrophobicity of the conformational variant compared to the (desired) polypeptide.

Due to said differences in biophysical parameters, the conformational variant to be converted and/or to be removed by the methods provided herein is distinguishable by chromatographic techniques such as analytical SE-HPLC and/or analytical IEX-HPLC. Accordingly, in one embodiment the conformational variant to be converted and/or to be removed by the methods provided herein is characterized by an increased retention time in SE-HPLC compared to the polypeptide. In another embodiment, the conformational variant is characterized by an altered retention time in IEX-HPLC compared to the polypeptide. In still another embodiment, the conformational variant is characterized by an increased retention time in SE-HPLC and an altered retention time in IEX-HPLC compared to the polypeptide.

In one aspect, the conformational variant is converted into the polypeptide by applying suitable conditions, wherein the conditions that convert the conformational variant into the polypeptide are selected from:
i) applying a low pH treatment in a step of the isolation and/or purification process;
ii) applying a chaotropic agent in a step of the isolation and/or purification process;
iii) applying a heat stress in a step of the isolation and/or purification process; or
iv) a combination of any of i) to iii).

The low pH treatment to convert the conformational variant into the polypeptide comprises decreasing the pH of a composition that comprises the conformational variant to about pH 3.2 or less, or to about pH 3.0 or less. In one aspect, the pH is decreased to between about pH 3.2 and about pH 2.1, to between about 3.0 and about pH 2.1, to between about pH 2.9 and about pH 2.1, to between about pH 2.7 and about pH 2.1, or to between about pH 2.6 and about pH 2.3. The pH treatment is applied for a sufficient amount of time to convert the conformational variant into the polypeptide. In view of the teaching provided in the present application the skilled person recognizes that the conversion of the conformational variant into the polypeptide increases over time. The conversion of the conformational variant into the polypeptide to a practical useful level is, however, already achieved after low pH treatment for at least 0.5 hours, such as for at least about 1 hour. Accordingly, in one aspect, the low pH treatment is applied for at least about 0.5 hours, for at least about 1 hour, for at least about 2 hours, or for at least about 4 hours. In a specific aspect, the pH is decreased to between about pH 3.2 and about pH 2.1, such as to about pH 3.2, 3.0, 2.9, 2.7, 2.5, 2.3, or 2.1. In another specific aspect, the pH is decreased to between about pH 3.0 and about pH 2.1, such as to about pH 3.0, 2.9, 2.7, 2.5, 2.3, or 2.1. In another specific aspect, the pH is decreased to between about pH 2.9 and about pH 2.1, such as to about pH, 2.9 2.7, 2.5, 2.3, or 2.1. In another specific aspect the pH is decreased to between about pH 2.5 and about pH 2.1, such as pH 2.5, pH 2.3, or pH 2.1. In another specific aspect, the pH is decreased to about pH 3.2 or less for at least 0.5 hours, such as for at least 1 hour. For instance, the pH is decreased to between about pH 3.2 and about 2.1 for at least about 0.5 hours, such as for at least about 1.0 hour. In still another aspect, the pH is decreased to about pH 3.0 or less for at least 0.5 hours, such as for at least 1 hour. For instance, the pH is decreased to between about pH 3.0 and about 2.1 for at least about 0.5 hours, such as for at least 1.0 hour. In still another aspect, the pH is decreased to about pH 2.9 or less for at least 0.5 hours, such as for at least 1 hour. For instance, the pH is decreased to between about pH 2.9 and about 2.1 for at least about 0.5 hours, such as for at least 1.0 hour. In still another aspect, the pH is decreased to about pH 2.7 or less for at least 0.5 hours, such as for at least 1 hour. For instance, the pH is decreased to between about pH 2.7 and about 2.1 for at least about 0.5 hours, such as for at least about 1.0 hour. In another aspect, the low pH treatment is terminated by increasing the pH used in the low pH treatment with at least one pH unit. In one embodiment, the polypeptide to be isolated/purified is obtainable by expression in Pichia, in particular P. pastoris.

In another specific aspect, the pH is decreased to about pH 2.5 or less for at least about 1 hour, or for at least about 2 hours. In another specific aspect, the pH is decreased to about pH 2.3 or less for at least about 1 hour. In another aspect, the low pH treatment is terminated by increasing the pH used in the low pH treatment with at least one pH unit. In one embodiment, the polypeptide to be isolated/purified is obtainable from expression in Pichia, in particular P. pastoris.

The low pH treatment used to convert the conformational variant into the polypeptide can be applied before or after a purification step based on a chromatographic technique. Before a purification step based on a chromatographic technique means that the low pH treatment is applied before the composition with the polypeptide to be purified is applied to the stationary phase of a chromatographic technique. After a purification step based on a chromatographic technique means that the low pH treatment is applied after the polypeptide to be purified is eluted from the stationary phase of a chromatographic technique. The stationary phase of a chromatographic technique is the chromatographic material used such as a chromatographic column comprising a resin or membrane. Accordingly, the low pH treatment can be applied after eluting the polypeptide from the stationary phase of the chromatographic technique used. The low pH treatment can be applied to the eluate obtained by a purification step based on a chromatographic technique. In this embodiment, the polypeptide is not bound to or eluting from (i.e., still in contact with) the stationary phase/chromatographic material of a chromatographic technique. After elution, the obtained eluate is then adjusted to the low pH treatment for a sufficient amount of time to convert the conformational variant into the polypeptide, as described herein. Accordingly, in an embodiment the low pH treatment is applied after elution of the polypeptide from the stationary phase of a purification step based on a chromatographic technique, i.e. to the eluate. In one embodiment, the polypeptide to be isolated/purified is obtainable by expression in Pichia, in particular P. pastoris.

The low pH treatment to convert the conformational variant into the polypeptide can be also applied during a purification step based on a chromatographic technique. During a purification step means that the low pH treatment is applied while the composition with the polypeptide to be purified is applied to the stationary phase of a chromatographic technique (i.e., the composition comprising the polypeptide to be purified is in contact with the stationary phase/chromatographic material of a chromatographic technique). During a purification step the composition with the polypeptide to be purified can be in contact with the stationary phase/chromatographic material (e.g., as in size exclusion chromatography) or can be (reversibly) bound to the stationary phase/chromatographic material (e.g., as in affinity chromatography). In one aspect, the elution buffer has a pH of equal to or less than pH 2.5. It is generally known, that the actual pH of an eluate is always higher than the initial pH of the low pH elution buffer. For instance, an elution with an elution buffer of pH 3.0 may result in an eluate pH of pH 3.8. The reason may be that remaining fluid present on the stationary phase of the chromatographic technique used and having a higher pH (e.g. buffer fluid used for storage, equilibration or recovery of the stationery phase or buffers used for binding the polypeptide to the stationary phase) mixes with the low pH buffer used in the low pH treatment during the purification step based on a chromatographic technique. Thus, alternatively, the elution buffer has a pH such that the resulting eluate containing the polypeptide has a pH of equal to or less than pH 2.9. In these aspects, the resulting eluate is optionally adjusted to a pH of equal to or less than pH 3.2, such as pH 2.7 for at least about 0.5 hours, such as for at least 1 hour. In one embodiment, the polypeptide to be isolated/purified is obtainable by expression in Pichia, in particular P. pastoris.

In view of the teaching provided in the present application, the skilled person recognizes that the conversion of the conformational variant into the polypeptide increases over time. The conversion of the conformational variant into the polypeptide to a practical useful level is, however, already achieved after low pH treatment for at least 0.5 hours, such as for at least about 1 hour. In one aspect, the pH of the eluate is decreased to about pH 3.2 or less for at least 0.5 hours, such as for at least 1 hour. For instance, the pH is decreased to between about pH 3.2 and about pH 2.1 for at least about 0.5 hours, such as for at least about 1.0 hour. In another aspect, the pH of the eluate is decreased to about pH 3.0 or less for at least 0.5 hours, such as for at least 1 hour. For instance, the pH is decreased to between about pH 3.0 and about pH 2.1 for at least about 0.5 hours, such as for at least about 1.0 hour. In still another aspect, the pH of the resulting eluate is decreased to about pH 2.9 or less for at least 0.5 hours, such as for at least 1 hour. For instance, the pH is decreased to between about pH 2.9 and about pH 2.1 for at least about 0.5 hours, such as for at least 1.0 hour. In still another aspect, the pH of the resulting eluate is decreased to about pH 2.7 or less for at least 0.5 hours, such as for at least 1 hour. For instance, the pH is decreased to between about pH 2.7 and about pH 2.1 for at least about 0.5 hours, such as for at least about 1.0 hour. Alternatively, the pH of the resulting eluate containing the polypeptide is decreased to a pH of equal to or less than pH 2.5. For instance, the pH is decreased to pH 2.7 or less for at least 0.5 hours, such as for at least 1 hour. In one embodiment, the polypeptide to be isolated/purified is obtainable by expression in Pichia, in particular P. pastoris.

In another aspect, the low pH treatment is terminated by increasing the pH used in the low pH treatment with at least one pH unit.

In another aspect, the low pH treatment to convert the conformational variant into the polypeptide is applied during a purification step based on Protein A-based affinity chromatography. In one embodiment, the polypeptide to be isolated/purified is obtainable by expression in Pichia, in particular P. Pastoris. In a specific aspect, the chromatographic technique is a Protein A-based affinity chromatography, wherein the elution buffer has a pH of about pH 2.2, and wherein the pH of the resulting eluate is adjusted to a pH of about pH 2.5 for at least about 1.5 hour.

In one aspect, the low pH treatment is terminated by increasing the pH to about pH 5.5 or higher. Moreover, in one aspect, the low pH treatment is applied after a purification step based on a chromatographic technique. Further, in one aspect, the low pH treatment is applied at room temperature.

In another aspect, a chaotropic agent is used to convert the conformational variant into the polypeptide. In one aspect, the chaotropic agent is guanidinium hydrochloride (GuHCl). In one aspect, the GuHCl is in a final concentration of least about 1 M, such as between about 1M and about 2M. In one aspect, the GuHCl is in a final concentration of at least about 2 M. The chaotropic agent treatment is applied for a sufficient amount of time to convert the conformational variant into the polypeptide. In one aspect, the GuHCl is applied for at least 0.5 hour, or for at least 1 hour. The chaotropic agent treatment is terminated by transferring the ISVD polypeptide product to a new buffer system lacking the chaotropic agent. In one aspect, the chaotropic agent treatment is applied after a purification step based on a chromatographic technique. In one aspect, the chaotropic agent is applied at room temperature. In one embodiment, the polypeptide to be isolated/purified is obtainable by expression in Pichia, in particular P. pastoris.

The heat stress applied to convert the conformational variant into the polypeptide comprises incubating a composition comprising the conformational variant between about 40° C. to about 60° C., between about 45° C. to about 60° C., or between to about 50° C. to about 60° C. The heat stress is applied for a sufficient amount of time to convert the conformational variant into the polypeptide. In one aspect, the heat stress is applied for at least about 1 hour. The heat stress is terminated by decreasing the temperature to room temperature. In one aspect, the heat stress is applied after a purification step based on a chromatographic technique. In one embodiment, the polypeptide to be isolated/purified is obtainable by expression in Pichia, in particular P. pastoris.

In another aspect, the conformational variant is converted into the polypeptide using a combination of the above conditions.

In another aspect, the conformational variant is removed from a composition comprising the multivalent polypeptide comprising at least three or at least four ISVDs by one or more chromatographic techniques. In one aspect, the chromatographic technique is a chromatographic technique based on hydrodynamic volume, surface charge or surface hydrophobicity. In one aspect, the chromatographic technique is size exclusion chromatography (SEC), ion-exchange chromatography (IEX), cation-exchange chromatography (CEX), mixed-mode chromatography (MMC), and/or hydrophobic interaction chromatography (HIC). In one embodiment, the polypeptide to be isolated/purified is obtainable by expression in Pichia, in particular P. pastoris.

In a further aspect, the conformational variant is removed by applying a composition comprising the multivalent polypeptide comprising at least three or at least four ISVDs to a chromatography column using a load factor of at least 20 mg protein/ml resin, at least 30 mg protein/ml resin, or at least 45 mg protein/ml resin. In one embodiment of this aspect, the chromatography column is a Protein A column. In one embodiment, the polypeptide to be isolated/purified is obtainable by expression in Pichia, in particular P. pastoris.

In another aspect, one or more of the conditions that convert the conformational variant into the polypeptide are applied alone, or in combination with one or more techniques that remove the conformational variant.

Also provided is a method of producing a polypeptide that comprises at least three or at least four immunoglobulin single variable domains (ISVDs), wherein the method comprises:

a) converting the conformational variant into the polypeptide by:
i) applying a low pH treatment in a step of the isolation and/or purification process;
ii) applying a chaotropic agent in a step of the isolation and/or purification process;
iii) applying a heat stress in a step of the isolation and/or purification process; or
iv) a combination of any of i) to iii),
   wherein the conditions are as further described herein;
   b) removing the conformational variant as further described herein; or
   c) a combination of a) and b).

In particular, following embodiments are provided:

Embodiment 1. A method of isolating or purifying a polypeptide that comprises or consists of at least three or at least four immunoglobulin single variable domains (ISVDs) from a composition comprising the polypeptide and a conformational variant thereof, the method comprising:
a) applying conditions that convert the conformational variant into the polypeptide;
b) removing the conformational variant; or
c) a combination of (a) and (b),
   optionally wherein the polypeptide to be isolated or purified is obtainable by expression in a host that is not a CHO cell.

Embodiment 2. The method according to embodiment 1, wherein the conformational variant results from expression of the polypeptide in a host that is not a CHO cell such as a lower eukaryotic host.

Embodiment 3: The method according to embodiment 1, wherein the polypeptide to be isolated or purified is obtainable by expression in a host that is a lower eukaryotic host.

Embodiment 4: The method according to embodiment 2 or embodiment 3, wherein the lower eukaryotic host is yeast such as Pichia, Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis.

Embodiment 5: The method according to embodiment 4, wherein the yeast is Pichia such as Pichia pastoris.

Embodiment 6. The method according to any one of embodiments 1 to 5, wherein the conformational variant is characterized by a more compact form compared to the polypeptide.

Embodiment 7. The method according to any one of embodiments 1 to 6, wherein the conformational variant has a decreased hydrodynamic volume compared to the polypeptide.

Embodiment 8. The method according to any of embodiments 1 to 7, wherein the conformational variant is characterized by an increased retention time in SE-HPLC compared to the polypeptide.

Embodiment 9. The method according to any of embodiments 1 to 8, wherein the conformational variant is characterized by an altered retention time in IEX-HPLC compared to the polypeptide.

Embodiment 10. The method according to embodiment 9, wherein the conformational variant is characterized by a decreased retention time in IEX-HPLC compared to the polypeptide.

Embodiment 11. The method according to embodiment 9, wherein the conformational variant is characterized by an increased retention time in IEX-HPLC compared to the polypeptide.

Embodiment 12. The method according to any one of embodiments 1 to 11, wherein the polypeptide comprises or consists of at least three ISVDs.

Embodiment 13. The method according to any one of embodiments 1 to 12, wherein the polypeptide comprises or consists of at least four ISVDs.

Embodiment 14. The method according to any of embodiments 1 to 11, wherein the polypeptide comprises or consists of three ISVDs, four ISVDs, or five ISVDs.

Embodiment 15. The method according to any of embodiments 1 to 14, wherein the conditions that convert the conformational variant into the polypeptide are selected from:
i) applying a low pH treatment in a step of the isolation and/or purification process, optionally wherein the low pH treatment comprises decreasing the pH of the composition to about pH 3.2 or less, or to about pH 3.0 or less;
ii) applying a chaotropic agent in a step of the isolation and/or purification process, optionally wherein the chaotropic agent is guanidinium hydrochloride (GuHCl);
iii) applying a heat stress in a step of the isolation and/or purification process, optionally comprising incubating the conformational variant at 40° C. to about 60° C.; or
iv) a combination of any of i) to iii),
   wherein any of the conditions is applied for a sufficient amount of time to convert the conformational variant into the polypeptide.

Embodiment 16: The method according to embodiment 15, wherein the polypeptide comprises or consists of at least four ISVDs, and the low pH treatment comprises decreasing the pH of the composition to about pH 3.0 or less.

Embodiment 17. The method according to embodiment 15 or embodiment 16, wherein the pH is decreased to between about pH 3.2 and about pH 2.1, to between about pH 3.0 and about pH 2.1, to between about pH 2.9 and about pH 2.1, to between about pH 2.7 and about pH 2.1, or to between about pH 2.6 and about pH 2.3.

Embodiment 18. The method according to embodiment 17, wherein the pH is decreased to about pH 3.0, to about pH 2.9, to about pH 2.8, to about pH 2.7, to about pH 2.6, to about pH 2.5, to about pH 2.4, to about pH 2.3, to about pH 2.2, or to about pH 2.1.

Embodiment 19. The method according to any of embodiments 15 to 18, wherein the low pH treatment is applied for at least about 0.5 hours, for at least about 1 hour, for at least about 2 hours, or for at least about 4 hours.

Embodiment 20. The method according to any of embodiments 15 to 19, wherein the pH is decreased to about pH 2.5 or less.

Embodiment 21. The method according to embodiments 15 to 19, wherein the pH is decreased to between about pH 3.0 and about pH 2.1 for at least 0.5 hours, for at least 1 hour, optionally for at least 2 hours.

Embodiment 22. The method according to embodiment 21, wherein the pH is decreased to between about pH 2.7 and about pH 2.1.

Embodiment 23. The method according to any of embodiments 15 to 19, wherein the pH is decreased to between about pH 2.7 and about pH 2.1 for at least 1 hour, optionally for at least 2 hours.

Embodiment 24. The method according embodiment 23, wherein the pH is decreased to between about pH 2.6 and about pH 2.3 for at least 1 hour, optionally for at least 2 hours.

Embodiment 25. The method according to any of embodiments 15 to 24, wherein the multivalent polypeptide comprises or consists of five ISVDs.

Embodiment 26. The method according to embodiment 25, wherein the pH is decreased to about pH 2.6 or less.

Embodiment 27. The method according to embodiment 25 or 26, wherein the low pH treatment is applied between 1 and 2 hours.

Embodiment 28. The method according to embodiment 27, wherein the polypeptide consists of SEQ ID NO: 1.

Embodiment 29. The method according to any of embodiments 15 to 24, wherein the multivalent polypeptide comprises or consists of four ISVDs.

Embodiment 30. The method according to embodiment 29, wherein the pH is decreased to about pH 2.9 or less, such as about pH 2.5.

Embodiment 31. The method according to embodiment 29 or 30, wherein the low pH treatment is applied between 1 and 2 hours.

Embodiment 32. The method according to embodiment 31, wherein the polypeptide consists of SEQ ID NO: 2.

Embodiment 33. The method according to embodiment 31, wherein the polypeptide consists of SEQ ID NO: 70 or SEQ ID NO:71.

Embodiment 34. The method according to any of embodiments 15 to 24, wherein the multivalent polypeptide comprises or consists of three ISVDs.

Embodiment 35. The method according to embodiment 34, wherein the pH is decreased to about pH 3.0 or less, such as about pH 2.5.

Embodiment 36. The method according to embodiment 34 or 35, wherein the low pH treatment is applied between 2 and 4 hours.

Embodiment 37. The method according to embodiment 36, wherein the polypeptide consists of SEQ ID NO: 69.

Embodiment 38. The method according to any of embodiments 15 to 37, wherein the low pH treatment is terminated by increasing the pH with at least one pH unit, with at least 2 pH units, or to about pH 5.5 or higher.

Embodiment 39. The method according to any of embodiments 15 to 38, wherein the low pH treatment is applied before or after a purification step based on a chromatographic technique.

Embodiment 40. The method according to embodiment 39, wherein the low pH treatment is applied before applying the composition to the stationary phase of a chromatographic technique.

Embodiment 41. The method according to embodiment 39, wherein the low pH treatment is applied after eluting the composition from the stationary phase of a chromatographic technique.

Embodiment 42. The method according to any of embodiments 15 to 38, wherein the low pH treatment is applied during a purification step based on a chromatographic technique, wherein the composition comprising the polypeptide to be purified is in contact with the stationary phase of a chromatographic technique.

Embodiment 43. The method according to embodiment 39 to 42, wherein the chromatographic technique is a Protein A-based affinity chromatography.

Embodiment 44. The method according to embodiment 43, wherein the chromatographic technique is a Protein A-based affinity chromatography, and wherein the elution buffer has a pH of equal to or less than pH 2.5.

Embodiment 45. The method according to embodiment 43, wherein the chromatographic technique is a Protein A-based affinity chromatography, and wherein the elution buffer has a pH such that the resulting eluate containing the polypeptide has a pH of equal to or less than pH 2.9.

Embodiment 46. The method according to any of embodiments 43 to 45, wherein the pH of the eluate containing the polypeptide is adjusted to a pH of equal to or less than pH 3.2, such as a pH of equal to or less than pH 3.0 or a pH equal to or less than pH 2.7, optionally for at least about 1 hour.

Embodiment 47. The method according to any of embodiments 43 to 45, wherein the pH of the eluate containing the polypeptide is adjusted to a pH of equal to or less than pH 2.5, optionally for at least about 1 hour.

Embodiment 48. The method according to embodiment 42, wherein the chromatographic technique is a Protein A-based affinity chromatography, wherein the elution buffer has a pH of about pH 2.2, and wherein the pH of the eluate containing the polypeptide is adjusted to a pH of about pH 2.5 for at least about 1.5 hour.

Embodiment 49. The method according to any of embodiments 42 to 48, wherein the pH of the eluate after the low pH treatment is increased with at least one pH unit, with at least two pH units, or to a pH of about pH 5.5 or higher.

Embodiment 50. The method according to any one of embodiments 15 to 49, wherein the low pH treatment is applied at room temperature.

Embodiment 51. The method according to any of embodiments 15 to 50, wherein the low pH treatment is followed by the steps of:
a) adding an appropriate amount of 1M sodium acetate pH 5.5 to the composition/eluate to obtain a final concentration of about 50 mM sodium acetate;
b) adjusting the pH of the composition/eluate to pH 5.5; and
c) adjusting the conductivity of the composition/eluate to about 6 mS/cm or lower using water.

Embodiment 52. The method according to embodiment 51, wherein the pH in b) is adjusted with NaOH.

Embodiment 53. The method according to embodiment 51 or 52, wherein the polypeptide comprises or consists of five ISVDs.

Embodiment 54. The method according to embodiment 51 or 52, wherein the polypeptide comprises or consists of four ISVDs.

Embodiment 55. The method according to embodiment 54, wherein the polypeptide consists of SEQ ID NO: 2.

Embodiment 56. The method according to any of embodiments 15 to 55, wherein GuHCl is applied in a final concentration of at least about 1 M, or at least about 2 M.

Embodiment 57. The method according to embodiments 15 to 56, wherein GuHCl is applied for at least 0.5 hours, or for at least 1 hour.

Embodiment 58. The method according to embodiment 56 or 57, wherein the GuHCl is applied in a final concentration of at least about 1M for at least 0.5 hours.

Embodiment 59. The method according to embodiment 58, wherein the GuHCl is applied in a final concentration of at least about 1M for 0.5 hours to 1 hour.

Embodiment 60. The method according to embodiment 56 or 57, wherein the GuHCl is applied in a final concentration of about 2M for at least 0.5 hours.

Embodiment 61. The method according to embodiment 60, wherein the GuHCl is applied in a final concentration of at least about 2M for 0.5 hours to 1 hour.

Embodiment 62. The method according to any one of embodiments 56 to 61, wherein the polypeptide comprises or consists of at least four ISVDs.

Embodiment 63. The method according to embodiment 61, wherein the polypeptide consists of SEQ ID NO: 1.

Embodiment 64. The method according to embodiment 61, wherein the polypeptide consists of SEQ ID NO: 2.

Embodiment 65. The method according to any one of embodiments 15 or 56 to 64, wherein the chaotropic agent treatment is applied at room temperature.

Embodiment 66. The method according to any of embodiments 15 or 56 to 65, wherein the chaotropic agent treatment is applied before or after a purification step based on a chromatographic technique.

Embodiment 67. The method according to embodiment 66, wherein the polypeptide is eluted from the stationary phase of the chromatographic technique and the chaotropic agent treatment is applied to the resulting eluate.

Embodiment 68. The method according to any of embodiments 15 to 67, wherein heat stress is applied for at least about 1 hour, or for about 1 to 4 hours.

Embodiment 69. The method according to embodiment 68, wherein the heat stress is applied at about 40° C. to about 60° C., at about 45° C. to about 60° C., or at about 50° C. to about 60° C.

Embodiment 70. The method according to embodiment 68, wherein the heat stress is applied at about 40° C. to about 55° C., at about 45° C. to 55° C., or at about 48° C. to about 52° C.

Embodiment 71. The method according to embodiment 68, wherein the heat stress is applied at about 50° C.

Embodiment 72. The method according to embodiment 71, wherein the heat stress is applied at about 50° C. for 1 hour.

Embodiment 73. The method according to embodiment 72, wherein the polypeptide comprises or consists of at least four ISVDs.

Embodiment 74. The method according to embodiment 72, wherein the polypeptide consists of SEQ ID NO: 1.

Embodiment 75. The method according to embodiment 72, wherein the polypeptide consists of SEQ ID NO: 2.

Embodiment 76. The method according to any of embodiments 15, or 68 to 75, wherein the heat stress is applied before or after a purification step based on a chromatographic technique.

Embodiment 77. The method according to embodiment 76, wherein the heat stress treatment is applied before applying the composition to the stationary phase of a chromatographic technique or after eluting the composition from the stationary phase of a chromatographic technique.

Embodiment 78. The method according to any of embodiments 1 to 14, wherein the conformational variant is removed by one or more chromatographic techniques.

Embodiment 79. The method according to embodiment 78, wherein the conformational variant has been identified by analytical chromatographic techniques such as SE-HPLC and IEX-HPLC before being removed by the one or more chromatographic techniques.

Embodiment 80. The method according to embodiment 78 or 79, wherein the chromatographic technique is a chromatographic technique based on hydrodynamic volume, surface charge or surface hydrophobicity.

Embodiment 81. The method according to embodiment 80, wherein the chromatographic technique is selected from any of size exclusion chromatography (SEC), ion-exchange chromatography (IEX), mixed-mode chromatography (MMC), and hydrophobic interaction chromatography (HIC).

Embodiment 82. The method according to embodiment 81, wherein the ion-exchange chromatography (IEX) is cation-exchange chromatography (CEX).

Embodiment 83. The method according to embodiment 81, wherein the HIC is based on a HIC column resin.

Embodiment 84. The method according to embodiment 83 wherein the HIC resin is selected from any of Capto Phenyl ImpRes, Capto Butyl ImpRes, Phenyl HP, and Capto Butyl.

Embodiment 85. The method according to embodiment 81, wherein the HIC is based on a HIC membrane.

Embodiment 86. The method according to any one of embodiments 1 to 85, wherein the composition is applied to a chromatography column using a load factor of at least 20 mg protein/ml resin, at least 30 mg protein/ml resin, at least 45 mg protein/ml resin, optionally wherein the chromatographic column is a Protein A column.

Embodiment 87. The method according to embodiment 86, wherein the composition is applied to a Protein A column using a load factor of at least 45 mg protein/ml resin.

Embodiment 88. The method according to embodiment 87, wherein the polypeptide consists of SEQ ID NO: 2.

Embodiment 89. The method according to any one of embodiments 1 to 88, wherein one or more of the conditions that convert the conformational variant into the polypeptide are applied alone, or in combination with one or more techniques that remove the conformational variant.

Embodiment 90. A method of isolating or purifying a polypeptide that comprises or consists of at least three or at least four immunoglobulin single variable domains (ISVDs), the method comprising one or more of the following:
i) applying a low pH treatment to a composition comprising the polypeptide in a step of the isolation or purification process, optionally wherein the low pH treatment comprises decreasing the pH of the composition to about pH 3.2 or less, or pH 3.0 or less;
ii) applying a chaotropic agent to a composition comprising the polypeptide in a step of the isolation or purification process, optionally wherein the chaotropic agent is GuHCl;
iii) applying a heat stress to a composition comprising the polypeptide in a step of the isolation or purification process, optionally comprising incubating the conformational variant at 40° C. to about 60° C.;
iv) applying the composition comprising the polypeptide to a chromatography column using a load factor of at least 20 mg/ml, at least 30 mg/ml, at least 45 mg/ml, optionally wherein the chromatographic column is Protein A column; or
v) a combination of any of i) to iv),
   optionally wherein the polypeptide to be isolated or purified is obtainable by expression in a host that is not a CHO cell.

Embodiment 91: The method according to embodiment 90, wherein the conformational variant results from expression of the polypeptide in a host that is not a CHO cell such as a lower eukaryotic host.

Embodiment 92. The method according to embodiment 90, wherein the polypeptide to be isolated or purified is obtainable by expression in a host that is a lower eukaryotic host.

Embodiment 93. The method according to embodiment 91 or embodiment 92, wherein the lower eukaryotic host is a yeast such as Pichia, Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis.

Embodiment 94. The method according to embodiment 93, wherein the yeast is Pichia such as Pichia pastoris.

Embodiment 95. The method according to any one of embodiments 90 to 94, wherein the pH is decreased to between about pH 3.2 and about pH 2.1, to between about pH 3.0 and about pH 2.1, between about pH 2.9 and about pH 2.1, to between about pH 2.7 and about pH 2.1, or to between about pH 2.6 and about pH 2.3.

Embodiment 96. The method according to embodiment 95, wherein the pH is decreased to about pH 3.0, to about pH 2.9, to about pH 2.8, to about pH 2.7, to about pH 2.6, to about pH 2.5, to about pH 2.4, to about pH 2.3, to about pH 2.2, or to about pH 2.1.

Embodiment 97. The method according to any of embodiments 90 to 96, wherein the low pH treatment is applied for at least about 0.5 hours, for at least about 1 hour, for at least about 2 hours, or for at least about 4 hours.

Embodiment 98. The method according to any of embodiments 90 to 97, wherein the pH is decreased to about pH 2.5 or less.

Embodiment 99. The method according to embodiments 90 to 97, wherein the pH is decreased to between about pH 3.0 and about pH 2.1 for at least 0.5 hours, for at least 1 hour or for at least 2 hours.

Embodiment 100. The method according to embodiment 99, wherein the pH is decreased to between about pH 2.7 and about pH 2.1.

Embodiment 101. The method according to any of embodiments 90 to 97, wherein the pH is decreased to between about pH 2.7 and about pH 2.1 for at least 1 hour, optionally for at least 2 hours.

Embodiment 102. The method according embodiment 101, wherein the pH is decreased to between about pH 2.6 and about pH 2.3 for at least 1 hour, optionally for at least 2 hours.

Embodiment 103. The method according to any of embodiments 90 to 102, wherein the multivalent polypeptide comprises or consists of three ISVDs, four ISVDs, or five ISVDs.

Embodiment 104. The method according to any one of embodiments 90 to 103, wherein the polypeptide comprises or consists of at least four ISVDs.

Embodiment 105. The method according to any one of embodiments 90 to 104, wherein the polypeptide comprises or consists of five ISVDs.

Embodiment 106. The method according to embodiment 105, wherein the pH is decreased to about pH 2.6 or less.

Embodiment 107. The method according to embodiment 103 to 106, wherein the low pH treatment is applied between 1 and 2 hours.

Embodiment 108. The method according to embodiment 107, wherein the polypeptide consists of SEQ ID NO: 1.

Embodiment 109. The method according to any of embodiments 90 to 104, wherein the multivalent polypeptide comprises or consists of four ISVDs.

Embodiment 110. The method according to embodiment 109, wherein the pH is decreased to about pH 2.9 or less, such as about pH 2.5.

Embodiment 111. The method according to embodiment 109 or 110, wherein the low pH treatment is applied between 1 and 2 hours.

Embodiment 112. The method according to embodiment 111, wherein the polypeptide consists of SEQ ID NO: 2.

Embodiment 113. The method according to embodiment 111, wherein the polypeptide consists of SEQ ID NO: 70 or SEQ ID NO 71.

Embodiment 114. The method according to any of embodiments 90 to 103, wherein the multivalent polypeptide comprises or consists of three ISVDs.

Embodiment 115. The method according to embodiment 114, wherein the pH is decreased to about pH 3.0 or less, such as about pH 2.5.

Embodiment 116. The method according to embodiment 114 or 115, wherein the low pH treatment is applied between 2 and 4 hours.

Embodiment 117. The method according to embodiment 116, wherein the polypeptide consists of SEQ ID NO: 69.

Embodiment 118. The method according to any of embodiments 90 to 117, wherein the low pH treatment is terminated by increasing the pH with at least one pH unit, with at least 2 pH units, or to about pH 5.5 or higher.

Embodiment 119. The method according to any of embodiments 90 to 118, wherein the low pH treatment is applied before or after a purification step based on a chromatographic technique.

Embodiment 120. The method according to embodiment 119, wherein the low pH treatment is applied before applying the composition to the stationary phase of a chromatographic technique.

Embodiment 121. The method according to embodiment 119, wherein the low pH treatment is applied after eluting the composition from the stationary phase of a chromatographic technique.

Embodiment 122. The method according to any of embodiments 90 to 118, wherein the low pH treatment is applied during a purification step based on a chromatographic technique, wherein the composition comprising the polypeptide to be purified is in contact with the stationary phase of a chromatographic technique.

Embodiment 123. The method according to embodiment 119 to 122, wherein the chromatographic technique is a Protein A-based affinity chromatography.

Embodiment 124. The method according to embodiment 123, wherein the chromatographic technique is a Protein A-based affinity chromatography, and wherein the elution buffer has a pH of equal to or less than pH 2.5.

Embodiment 125. The method according to embodiment 123, wherein the chromatographic technique is a Protein A-based affinity chromatography, and wherein the elution buffer has a pH such that the resulting eluate containing the polypeptide has a pH of equal to or less than pH 2.9.

Embodiment 126. The method according to any of embodiments 123 to 125, wherein the pH of the eluate containing the polypeptide is adjusted to a pH of equal to or less than 3.0, optionally for at least 1 hour, such as to a pH of equal to or less than pH 2.7, optionally for at least 0.5 hours or about 1 hour.

Embodiment 127. The method according to any of embodiments 123 to 125, wherein the pH of the eluate containing the polypeptide is adjusted to a pH of equal to or less than pH 2.5, optionally for at least about 0.5 hours or 1 hour.

Embodiment 128. The method according to embodiment 122, wherein the chromatographic technique is a Protein A-based affinity chromatography, wherein the elution buffer has a pH of about pH 2.2, and wherein the pH of the eluate containing the polypeptide is adjusted to a pH of about pH 2.5 for at least about 1.5 hour.

Embodiment 129. The method according to any of embodiments 119 to 128, wherein the pH of the eluate after the low pH treatment is increased for at least one pH unit, for at least two pH units, or to a pH of about pH 5.5 or higher.

Embodiment 130. The method according to any one of embodiments 90 to 129, wherein the low pH treatment is applied at room temperature.

Embodiment 131. The method according to any of embodiments 90 to 130, wherein the low pH treatment is followed by the steps of:
a) adding an appropriate amount of 1M sodium acetate pH 5.5 to the composition/eluate to obtain a final concentration of about 50 mM sodium acetate;
b) adjusting the pH of the composition/eluate to pH 5.5; and
c) adjusting the conductivity of the composition/eluate to about 6 mS/cm or lower using water.

Embodiment 132. The method according to embodiment 131, wherein the pH in b) is adjusted with NaOH.

Embodiment 133. The method according to embodiment 131 or 132, wherein the polypeptide comprises or consists of five ISVDs.

Embodiment 134. The method according to embodiment 131 or 132, wherein the polypeptide comprises or consists of four ISVDs.

Embodiment 135. The method according to embodiment 134, wherein the polypeptide consists of SEQ ID NO: 2.

Embodiment 136. The method according to any of embodiments 90 to 135, wherein GuHCl is applied in a final concentration of at least about 1 M, or at least about 2 M.

Embodiment 137. The method according to embodiments 90 or 136, wherein the GuHCl is applied for at least 0.5 hours, or for at least 1 hour.

Embodiment 138. The method according to embodiment 136 or 137, wherein the GuHCl is applied in a final concentration of at least about 1M for at least 0.5 hours.

Embodiment 139. The method according to embodiment 138, wherein the GuHCl is applied in a final concentration of at least about 1M for 0.5 hours to 1 hour.

Embodiment 140. The method according to embodiment 136 or 137, wherein the GuHCl is applied in a final concentration of about 2M for at least 0.5 hours.

Embodiment 141. The method according to embodiment 140, wherein the GuHCl is applied in a final concentration of at least about 2M for 0.5 hours to 1 hour.

Embodiment 142. The method according to any one of embodiments 90 or 136 to 141, wherein the polypeptide comprises or consists of at least four ISVDs.

Embodiment 143. The method according to embodiment 142, wherein the polypeptide consists of SEQ ID NO: 1.

Embodiment 144. The method according to embodiment 142, wherein the polypeptide consists of SEQ ID NO: 2.

Embodiment 145. The method according to any one of embodiments 90 or 136 to 144, wherein the chaotropic agent treatment is applied at room temperature.

Embodiment 146. The method according to any of embodiments 90 or 136 to 145, wherein the chaotropic agent treatment is applied before or after a purification step based on a chromatographic technique.

Embodiment 147. The method according to embodiment 146, wherein the polypeptide is eluted from the stationary phase of the chromatographic technique and the chaotropic agent treatment is applied to the resulting eluate.

Embodiment 148. The method according to embodiment 90 to 147, wherein heat stress is applied for at least about 1 hour, or for about 1 to 4 hours.

Embodiment 149. The method according to embodiment 148, wherein the heat stress is applied at about 40° C. to about 60° C., at about 45° C. to about 60° C., or at about 50° C. to about 60° C.

Embodiment 150. The method according to embodiment 148, wherein the heat stress is applied at about 40° C. to about 55° C., at about 45° C. to 55° C., or at about 48° C. to about 52° C.

Embodiment 151. The method according to embodiment 148, wherein the heat stress is applied at about 50° C.

Embodiment 152. The method according to embodiment 151, wherein the heat stress is applied at about 50° C. for 1 hour.

Embodiment 153. The method according to any one of embodiments 148 to 152, wherein the polypeptide comprises or consists of at least four ISVDs.

Embodiment 154. The method according to embodiment 152, wherein the polypeptide consists of SEQ ID NO: 1.

Embodiment 155. The method according to embodiment 152, wherein the polypeptide consists of SEQ ID NO: 2.

Embodiment 156. The method according to any of embodiments 90 or 148 to 155, wherein the heat stress is applied before or after a purification step based on a chromatographic technique.

Embodiment 157. The method according to embodiment 156, wherein the heat stress treatment is applied before applying the composition to the stationary phase of a chromatographic technique or after eluting the composition from the stationary phase of a chromatographic technique.

Embodiment 158. A method of producing a polypeptide that comprises at least three or at least four immunoglobulin single variable domains (ISVDs), wherein the method comprises the purification and/or isolation of the polypeptide according to any of the methods of embodiments 1 to 154.

Embodiment 159. The method according to embodiment 158, at least comprising the following steps:
a) optionally cultivating a host or host cell under conditions that are such that the host or host cell will multiply;
b) maintaining the host or host cell under conditions that are such that the host or host cell expresses and/or produces said polypeptide; and
c) isolating and/or purifying the secreted polypeptide from the medium comprising one or more of the isolation or purification methods according to any of embodiments 1 to 154,
optionally wherein the host is not a CHO cell.

Embodiment 160. The method according to embodiment 158 or 159, wherein the host is a lower eukaryotic host.

Embodiment 161. The method according to embodiment 160, wherein the lower eukaryotic host is a yeast such as Pichia, Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis.

Embodiment 162. The method according to embodiment 161, wherein the yeast is Pichia such as Pichia pastoris.

Embodiment 163. A method for isolating or purifying a polypeptide that comprises or consists of at least three or at least four immunoglobulin single variable domains (ISVDs) from a composition comprising the polypeptide and a conformational variant thereof, the method comprising:
(1) Identifying the conformational variant by analytical chromatographic techniques such as SE-HPLC and IEX-HPLC;
(2) Adjusting the chromatographic conditions to allow specific removal of the conformational variant; and
(3) Removing the conformational variant from the composition comprising the polypeptide and the conformational variant thereof by one or more chromatographic techniques,
   optionally wherein the polypeptide to be isolated or purified is obtainable by expression in a host that is not a CHO cell.

Embodiment 164. A method for optimizing one or more chromatographic techniques to allow isolation or purification of a polypeptide that comprises or consists of at least three or at least four immunoglobulin single variable domains (ISVDs) from a composition comprising the polypeptide and a conformational variant thereof, the method comprising:
(1) Identifying the conformational variant by analytical chromatographic techniques such as SE-HPLC and IEX-HPLC;
(2) Optimizing the chromatographic conditions to allow specific removal of the conformational variant,
   optionally wherein the polypeptide to be isolated or purified is obtainable by expression in a host that is not a CHO cell.

Embodiment 165. The method according to embodiment 163 or 164, wherein the conformational variant results from expression of the polypeptide in a host that is not a CHO cell such as a lower eukaryotic host.

Embodiment 166: The method according to embodiment 163 or 164, wherein the polypeptide to be isolated or purified is obtainable by expression in a host that is a lower eukaryotic host.

Embodiment 167: The method according to embodiment 165 or embodiment 166, wherein the lower eukaryotic host is yeast such as Pichia, Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis.

Embodiment 168: The method according to embodiment 167, wherein the yeast is Pichia such as Pichia pastoris.

Embodiment 169. The method according to any one of embodiments 163 to 168, wherein the conformational variant is characterized by a more compact form compared to the polypeptide.

Embodiment 170. The method according to any one of embodiments 163 to 169, wherein the conformational variant has a decreased hydrodynamic volume compared to the polypeptide.

Embodiment 171. The method according to any of embodiments 163 to 170, wherein the conformational variant is characterized by an increased retention time in SE-HPLC compared to the polypeptide.

Embodiment 172. The method according to any of embodiments 163 to 171, wherein the conformational variant is characterized by an altered retention time in IEX-HPLC compared to the polypeptide.

Embodiment 173. The method according to embodiment 172, wherein the conformational variant is characterized by a decreased retention time in IEX-HPLC compared to the polypeptide.

Embodiment 174. The method according to embodiment 172, wherein the conformational variant is characterized by an increased retention time in IEX-HPLC compared to the polypeptide.

Embodiment 175. The method according to any one of embodiments 163 to 174, wherein the polypeptide comprises or consists of at least three ISVDs.

Embodiment 176. The method according to any one of embodiments 163 to 175, wherein the polypeptide comprises or consists of at least four ISVDs.

Embodiment 177. The method according to any of embodiments 163 to 176, wherein the polypeptide comprises or consists of three ISVDs, four ISVDs, or five ISVDs.

Embodiment 178. The method according to any one of embodiments 163 to 177, wherein the chromatographic technique is a chromatographic technique based on hydrodynamic volume, surface charge or surface hydrophobicity.

Embodiment 179. The method according to embodiment 178, wherein the chromatographic technique is selected from any of size exclusion chromatography (SEC), ion-exchange chromatography (IEX), mixed-mode chromatography (MMC), and hydrophobic interaction chromatography (HIC).

Embodiment 180. The method according to embodiment 179, wherein the ion-exchange chromatography (IEX) is cation-exchange chromatography (CEX).

Embodiment 181. The method according to embodiment 179, wherein the HIC is based on a HIC column resin.

Embodiment 182. The method according to embodiment 181 wherein the HIC resin is selected from any of Capto Phenyl ImpRes, Capto Butyl ImpRes, Phenyl HP, and Capto Butyl.

Embodiment 183. The method according to embodiment 179, wherein the HIC is based on a HIC membrane.

### 4 DESCRIPTION OF THE FIGURES

FIG. 1: SE-HPLC chromatograms (incl. zoom, lower panel) of eluates post capture using protein A or non-protein A capture resins.

FIG. 2: SE-HPLC chromatograms (incl. zoom, lower panel) of eluates post protein A capture with elution buffer A, B, C and D as described in Table 2.

FIG. 3: SE-HPLC chromatograms (incl. zoom, lower panel) of eluates post protein A capture with elution buffer A in (1) and elution buffer B in (2) with or without pH neutralization.

FIG. 4: Chromatographic profile of compound A on a cation exchange resin used for polish development.

FIG. 5: SE-HPLC chromatograms (incl. zoom, lower panel) of load, side and top fraction obtained in the preparative CEX as described in Example 1 and FIG. 4.

FIG. 6: IEX-HPLC chromatograms (incl. zoom, lower panel) of conformational variant-enriched side fraction and conformational variant-depleted top fraction obtained in the preparative CEX as described in Example 1 and FIG. 4.

FIG. 7: SE-HPLC chromatograms (incl. zooms, lower panel) after low pH treatment (pH 2.5) of conformational variant-enriched (1) and -depleted material (2).

FIG. 8: IEX-HPLC chromatogram (incl. zoom, lower panel) after low pH treatment (pH 2.5) of conformational variant-enriched material.

FIG. 9: SE-HPLC chromatograms (incl. zoom, lower panel) of conformational variant-enriched material treated with 2M or 3M GuHCl chaotropic agent for 0.5 hours at RT.

FIG. 10: IEX-HPLC chromatograms (incl. zoom, lower panel) of conformational variant-enriched material treated with 2M or 3M GuHCl chaotropic agent treatment for 0.5 hours at RT.

FIG. 11: SE-HPLC chromatogram (incl. zoom, lower panel) of conformational variant-enriched material treated at 50° C. for 1 hour.

FIG. 12: IEX-HPLC chromatogram (incl. zoom, lower panel) of conformational variant-enriched material treated at 50° C. for 1 hour.

FIG. 13: SE-HPLC chromatograms (incl. zoom, lower panel) of the capture eluates using different elution conditions, as described in Example 4.

FIG. 14: IEX-HPLC chromatograms (incl. zoom, lower panel) of the capture eluates using different elution conditions, as described in Example 4.

FIG. 15: SE-HPLC chromatograms (incl. zoom, lower panel) of the capture eluate after low pH incubation and immediate pH adjustment post low pH (T0) in (1) and (2); and after low pH incubation and pH adjustment after 1 h incubation at low pH (T1 h) in (3) and (4).

FIG. 16A: SE-HPLC chromatograms (incl. zoom, lower panel) of the samples after application of two different sets of pH adjustment stock solutions.

FIG. 16B: Influence of pH on the product quality of compound A analyzed by IEX-HPLC as described in Example 4 (first experiment).

FIG. 16C: Influence of pH on the product quality of compound A analyzed by IEX-HPLC as described in Example 4 (second experiment).

FIG. 17: SE-HPLC chromatograms (incl. zoom, lower panel) of capture eluate and capture filtrate from 10 L scale (1) and 100 L scale (2).

FIG. 18: IEX-HPLC chromatograms (incl. zoom, lower panel) of capture eluate and capture filtrate from 10 L scale.

FIG. 19: IEX-HPLC chromatograms of capture eluate and capture filtrate from 100 L scale.

FIG. 20: Chromatographic MMC profile used for the removal of the conformational variant of compound A. In grey boxes: fractions F8 and F11 selected for analysis.

FIG. 21: SE-HPLC chromatograms (incl. zoom, lower panel) of load and fraction F8 in (1) and of load and fraction F11 in (2) obtained in MMC as described in Example 6.

FIG. 22: IEX-HPLC chromatograms (incl. zoom, lower panel) of load and fraction F8 in (1) and of load and fraction F11 in (2) obtained in MMC as described in Example 6.

FIG. 23: Chromatographic HIC profile on TSK Phenyl gel 5 PW(30) resin used for removal of the conformational variant of compound A. In grey boxes: fractions F26 and F41 selected for analysis.

FIG. 24: SE-HPLC chromatograms (incl. zoom, lower panel) of load and fraction F26 in (1) and of load and fraction F41 in (2) obtained in HIC with TSK Phenyl gel 5 PW(30) resin.

FIG. 25: SE-HPLC chromatograms (incl. zoom, lower panel) of the top fraction and load obtained in HIC with Capto Butyl Impres resin used with an ammonium sulphate gradient.

FIG. 26: Chromatographic HIC profile on Capto Butyl ImpRes resin used for removal of the conformational variant of compound A. In grey boxes: fractions F15, F20, and F29 selected for analysis.

FIG. 27: SE-HPLC chromatograms (incl. zoom, lower panel) of load and fractions F15, F20 and F29 obtained in HIC with Capto Butyl ImpRes resin.

FIG. 28: SE-HPLC chromatograms (incl. zoom, lower panel) of capture eluate after membrane-based HIC on Sartobind Phenyl membrane (filter plate).

FIG. 29: Chromatographic HIC profile on Sartobind Phenyl membrane used for removal of the conformational variant of compound A.

FIG. 30: SE-HPLC chromatograms (incl. zoom, lower panel) of the load, fraction pool 2, and strip fraction obtained in HIC on Sartobind Phenyl membrane.

FIG. 31: IEX-HPLC chromatogram (incl. zoom, lower panel) of compound B.

FIG. 32: Chromatographic CEX profile of compound B during the polish process step. In grey boxes: fractions selected for analysis.

FIG. 33: IEX-HPLC chromatograms (incl. zoom, lower panel) of fraction 2C4 and pool fractions 2C7-2C11 obtained in CEX as described in Example 7.

FIG. 34: SE-HPLC chromatograms (incl. zoom, lower panel) of fraction 2C4 and pool fractions 2C7-2C11 obtained in CEX as described in Example 7.

FIG. 35: IEX-HPLC chromatograms (incl. zoom, lower panel) of capture eluate of compound B after low pH treatment at pH 2.3 for 1 hour and subsequent adjustment to pH 5.5 with 1M sodium acetate. Capture eluate directly adjusted to pH 5.5 with 1M sodium acetate was used as control.

FIG. 36: SE-HPLC chromatograms (incl. zoom, lower panel) of capture eluate of compound B after low pH treatment at pH 2.3 for 1 hour and subsequent adjustment to pH 5.5 with 1M sodium acetate. Capture eluate directly adjusted to pH 5.5 with 1M sodium acetate was used as control.

FIG. 37: IEX-HPLC chromatogram (incl. zoom, lower panel) of the capture eluate of compound B following low pH 2.5 treatment for 4 h.

FIG. 38: SE-HPLC chromatogram (incl. zoom, lower panel) of the capture eluate of compound B following low pH 2.5 treatment for 4 h.

FIG. 39: IEX-HPLC chromatograms (incl. zoom, lower panel) of the capture eluate of compound B following 0.5 h GuHCl chaotropic agent treatment at RT.

FIG. 40: IEX-HPLC chromatograms (incl. zoom, lower panel) of the capture eluate of compound B following 1 h heat treatment at 50° C.

FIG. 41: SE-HPLC chromatograms (incl. zoom, lower panel) of the capture eluate of compound B following 1 h heat treatment at 50° C.

FIG. 42A: SE-HPLC chromatograms (incl. zoom, lower panel) of the capture eluate of compound B following treatment at pH 2.3 and subsequent adjustment to pH 5.5 directly or after 1 h.

FIG. 42B: SE-HPLC chromatograms (incl. zoom, lower panel) of the capture eluate of compound B following treatment at pH 2.5 and subsequent adjustment to pH 5.5 directly or after 1 h.

FIG. 43: Influence of the low pH treatment on the product quality analysed in function of time by IEX-HPLC. (A) initial experiment with low pH treatment at pH 2.3 and pH 2.5 for 2 and 4 hours; (B) additional experiment with low pH treatment at pH 2.7, pH 2.9, pH 3.1, pH 3.3, pH 3.5 and pH 2.7; for 2 and 4 hours.

FIG. 44: SE-HPLC chromatograms (incl. zoom, lower panel) of the capture eluate of compound B following treatment at pH 2.4 and pH 2.6 for 2 h and subsequent adjustment to pH 5.5.

FIG. 45: SE-HPLC chromatogram (incl. zoom, lower panel) of capture eluate of compound B following treatment at pH 2.6 for 2 h and subsequent adjustment to pH 5.5.

FIG. 46 Chromatographic CEX profile used for removal of the conformational variant of compound B. In grey boxes: fractions selected for analysis.

FIG. 47: Chromatographic HIC profile on Capto Butyl ImpRes resin used for the removal of the conformational variant of compound B. In grey boxes: fractions selected for analysis.

FIG. 48: SDS-PAGE analysis of selected fractions of a HIC run on Capto Butyl ImpRes as depicted in FIG. 47.

FIG. 49: Prediction profiler of the DOE model representing the impact of the load factor on product quality as assessed by IEX-HPLC analysis.

FIG. 50: SE-HPLC chromatograms (incl. zoom, lower panel) of representative capture eluate of cycle 1 and representative capture filtrate of cycle 1 from the 10 L scale-up.

FIG. 51: SE-HPLC chromatograms (incl. zoom, lower panel) of representative capture eluate of cycle 1 and representative capture filtrate of cycle 1 from the 100 L scale-up.

FIG. 52: Schematic representation of the hypothesized model.

FIG. 53: (A) SE-HPLC chromatograms (incl. zoom, lower panel) of the capture eluate of compound C produced in P. pastoris following low pH 3.0 treatment for 0 h, 2 h, and 4 h. (B) SE-HPLC chromatograms (incl. zoom, lower panel) of the capture eluate of compound C following low pH 2.5 treatment for 0 h, 2 h, and 4 h.

FIG. 54: Influence of pH on the product quality of compound C analyzed by SE-HPLC as described in Example 14.

FIG. 55: SE-HPLC chromatograms (incl. zoom, lower panel) of the capture eluate of compound C produced in CHO cells following low pH treatment at pH 2.6 and pH 3.0 compared to treatment at pH 5.5 after 2 h incubation.

FIG. 56: The influence of pH on the product quality of compound D analyzed by SE-HPLC as described in Example 16.

FIG. 57: The influence of pH on the product quality of compound E analyzed by SE-HPLC as described in Example 17.

### 5 DETAILED DESCRIPTION

The present disclosure describes the surprising observation of a conformational variant of a polypeptide comprising or consisting of at least three or at least four immunoglobulin single variable domains (ISVDs). A conformational variant of said polypeptide was observed during the production of the polypeptide in a host. In particular, the conformational variant was observed upon production of the polypeptide comprising or consisting of at least three or at least four ISVDs in a host, such as in a lower eukaryotic host as described herein. It could be revealed that the conformational variant of the multivalent polypeptide product comprising at least three or at least four ISVDs results from expression of the polypeptide in a host, in particular in a host that is a lower eukaryotic host such as yeast. The molecular weight of the polypeptide and its conformational variant are the same, but the conformational variant displayed a change in charge/surface characteristics leading to a different physico-chemical behaviour e.g., different retention time on analytical size exclusion chromatography and/or analytical ion exchange chromatography. Accordingly, the conformational variant of the polypeptide comprising or consisting of at least three or at least four ISVDs could be observed as a shoulder post peak or a resolved post peak of the polypeptide-containing main peak on analytical size exclusion chromatography (SE-HPLC post peak 1) and/or as a post peak shoulder or a resolved post peak of the polypeptide-containing main peak in analytical ion exchange chromatography (IEX-HPLC post peak 1). Such different physico-chemical behaviour was not due to scrambled disulfide bridges.

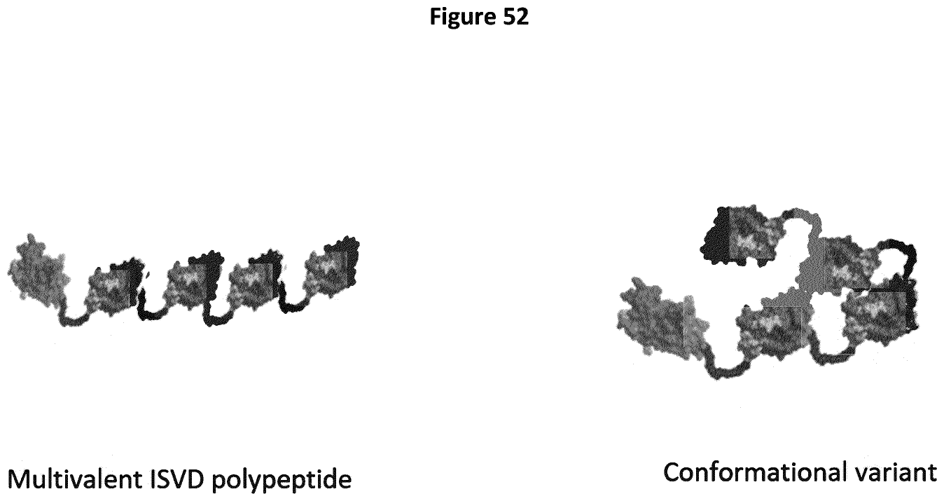

Based on these observations, it was hypothesized that a polypeptide comprising or consisting of at least three or at least four ISVDs allows a certain structural flexibility leading to intramolecular interactions such that the polypeptide can occur as a conformational variant that has a conformational arrangement of the ISVD building blocks that results in a more compact form compared to the arrangement of the ISVD building blocks of the polypeptide (see FIG. 52). Although an ISVD per se is a very stable molecule, it was surprisingly observed that increasing the valency of a polypeptide to at least three or at least four ISVDs (i.e., increasing the number of ISVD building blocks to three, four or more) may render the polypeptide more prone to intramolecular interactions. Without being bound by hypothesis, it was concluded that a polypeptide comprising or consisting of at least three or at least four ISVDs can allow the intramolecular interaction between at least two ISVDs within said polypeptide forming a conformational variant of the polypeptide having a compact form. The compact form is characterized by a decreased hydrodynamic volume compared to the polypeptide. Moreover, it was found that the compact form can be characterized by an altered surface charge and/or an altered surface hydrophobicity/hydrophobicity exposure. Accordingly, the polypeptide comprising or consisting of at least three or at least four ISVDs and the conformational variant thereof can be distinguished based on analytical chromatographic techniques. In particular, the polypeptide comprising or consisting of at least three or at least four ISVDs and the conformational variant thereof can be distinguished based on shifts in hydrodynamic volume and/or surface charge by analytical chromatographic techniques such as size exclusion high-performance liquid chromatography (SE-HPLC), and/or ion-exchange high-performance liquid chromatography (IEX-HPLC).

It was further demonstrated that the conformational variant can be converted into the (desired) polypeptide using the treatment conditions revealed in this application. Moreover, it was found that based on the observed biochemical/biophysical differences between the polypeptide and the conformational variant thereof, the conformational variant can be removed from a composition comprising the polypeptide and the conformational variant thereof using known preparative chromatographic techniques based on hydrodynamic volume, surface charge and/or surface hydrophobicity, as described herein.

### 5.1 Definitions

Unless indicated or defined otherwise, all terms used have their usual meaning in the art, which will be clear to the skilled person. Reference is for example made to the standard handbooks, such as Sambrook et al. 1989 (Molecular Cloning: A Laboratory Manual, 2nd Ed., Vols. 1-3, Cold Spring Harbor Laboratory Press), Ausubel et al. 1987 (Current protocols in molecular biology, Green Publishing and Wiley Interscience, New York), Lewin 1985 (Genes II, John Wiley & Sons, New York, N.Y.), Old et al. 1981 (Principles of Gene Manipulation: An Introduction to Genetic Engineering, 2nd Ed., University of California Press, Berkeley, Calif.), Roitt et al. 2001 (Immunology, 6th Ed., Mosby/Elsevier, Edinburgh), Roitt et al. 2001 (Roitt's Essential Immunology, 10th Ed., Blackwell Publishing, UK), and Janeway et al. 2005 (Immunobiology, 6th Ed., Garland Science Publishing/Churchill Livingstone, N.Y.), as well as to the general background art cited herein.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews: Presta 2006 (Adv. Drug Deliv. Rev. 58: 640), Levin and Weiss 2006 (Mol. Biosyst. 2: 49), Irving et al. 2001 (J. Immunol. Methods 248: 31), Schmitz et al. 2000 (Placenta 21 Suppl. A: S106), Gonzales et al. 2005 (Tumour Biol. 26: 31), which describe techniques for protein engineering, such as affinity maturation and other techniques for improving the specificity and other desired properties of proteins such as immunoglobulins.

The term “about” used in the context of the parameters or parameter ranges provided herein shall have the following meanings. Unless indicated otherwise, where the term “about” is applied to a particular value or to a range, the value or range is interpreted as being as accurate as the method used to measure it. If no error margins are specified in the application, the last decimal place of a numerical value indicates its degree of accuracy. Where no other error margins are given, the maximum margin is ascertained by applying the rounding-off convention to the last decimal place, e.g. for a pH value of about pH 2.7, the error margin is 2.65-2.74. However, for the following parameters, the specific margins shall apply: a temperature specified in ° C. with no decimal place shall have an error margin of ±1° C. (e.g., a temperature value of about 50° C. means 50° C.±1° C.); a time indicated in hours shall have an error margin of 0.1 hours irrespective of the decimal places (e.g., a time value of about 1.0 hours means 1.0 hours±0.1 hours; a time value of about 0.5 hours means 0.5 hours±0.1 hours).

In the present application, any parameter indicated with the term “about” is also contemplated as being disclosed without the term “about”. In other words, embodiments referring to a parameter value using the term “about” shall also describe an embodiment directed to the numerical value of said parameter as such. For example, an embodiment specifying a pH of “about pH 2.7” shall also disclose an embodiment specifying a pH of “pH 2.7” as such; an embodiment specifying a pH range of “between about pH 2.7 and about pH 2.1” shall also describe an embodiment specifying a pH range of “between pH 2.7 and pH 2.1”, etc.

### 5.2 Immunoglobulin Single Variable Domains

The term “immunoglobulin single variable domain” (ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ablz, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain.

The binding site of an immunoglobulin single variable domain is formed by a single V_{H}, a single V_{HH} or single V_{L} domain.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy chain ISVD, such as a V_{H}, V_{HH}, including a camelized V_{H} or humanized V_{HH}. In one embodiment, it is a V_{HH}, including a camelized V_{H} or humanized V_{HH}. Heavy-chain ISVDs can be derived from a conventional four-chain antibody or from a heavy chain antibody.

For example, the immunoglobulin single variable domain may be a single domain antibody (or an amino acid sequence that is suitable for use as a single domain antibody), a “dAb” or dAb (or an amino acid sequence that is suitable for use as a dAb) or a NANOBODY® ISVD (as defined herein, and including but not limited to a V_{HH}); other single variable domains, or any suitable fragment of any one thereof.

In particular, the immunoglobulin single variable domain may be a NANOBODY® ISVD (such as a V_{HH}, including a humanized V_{HH} or camelized V_{H}) or a suitable fragment thereof. [Note: NANOBODY® is a registered trademark of Ablynx N.V.]

“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. Nature 363: 446-448, 1993). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans (Reviews in Molecular Biotechnology 74: 277-302, 2001).

Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries e.g. by phage display.

The generation of immunoglobulin sequences, such as VHHs, has been described extensively in various publications, among which WO 94/04678, Hamers-Casterman et al. 1993 and Muyldermans et al. 2001 (Reviews in Molecular Biotechnology 74: 277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production.

Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be produced, purified and/or isolated in the method described herein. Also, fully human, humanized or chimeric sequences can be produced, purified and/or isolated in the method described herein. For example, camelid immunoglobulin sequences and humanized camelid immunoglobulin sequences, or camelized domain antibodies, e.g. camelized dAb as described by Ward et al (see for example WO 94/04678 and Riechmann, Febs Lett., 339:285-290, 1994 and Prot. Eng., 9:531-537, 1996) can be produced, purified and/or isolated in the method described herein. Moreover, the ISVDs are fused to comprise or consist of at least three or at least four ISVDs forming a multivalent and/or multispecific construct (for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221). ISVD sequences may comprise tags or other functional moieties, e.g. toxins, labels, radiochemicals, etc.

A “humanized V_{HH}^{”} comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art (e.g. WO 2008/020079). Again, it should be noted that such humanized V_{HH}s can be obtained in any suitable manner known per se and thus are not strictly limited to polypeptides that have been obtained using a polypeptide that comprises a naturally occurring V_{HH} domain as a starting material.

A “camelized V_{H}^{”} comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a (camelid) heavy chain antibody. This can be performed in a manner known per se, which will be clear to the skilled person, for example on the basis of the further description herein and the prior art (e.g. Davies and Riechmann (1994 and 1996), supra). Such “camelizing” substitutions are inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). In one embodiment, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

It should be noted that one or more ISVD sequences may be linked to each other and/or to other amino acid sequences (e.g. via disulphide bridges) to provide peptide constructs that may also be useful in the present method (for example Fab′ fragments, F(ab′)2 fragments, scFv constructs, “diabodies” and other multispecific constructs). Reference is for example made to the review by Holliger and Hudson, Nat Biotechnol. 2005 September; 23(9):1126-36)). Generally, when a polypeptide is intended for administration to a subject (for example for prophylactic, therapeutic and/or diagnostic purposes), it comprises an immunoglobulin sequence that does not occur naturally in said subject.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

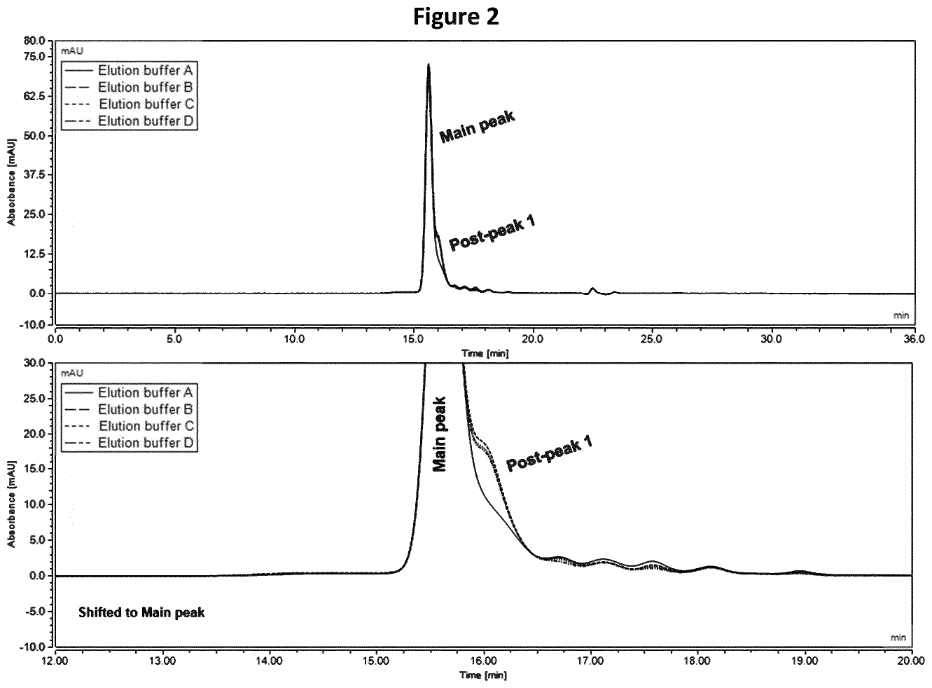

As further described in paragraph q) on pages 58 and 59 of WO 08/020079 (incorporated herein by reference), the amino acid residues of an immunoglobulin single variable domain can be numbered according to the general numbering for V_{H} domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, 2000 (J. Immunol. Methods 240 (1-2): 185-195; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

CDR sequences can be determined according to the AbM numbering as described in Kontermann and Dübel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

Determination of CDR regions may also be done according to different methods. In the CDR determination according to Kabat, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence or V_{HH} sequence). In one particular aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence used in the methods described herein may contain one or more of hallmark residues (as defined herein), such that the ISVD sequence is a NANOBODY® ISVD, such as a V_{HH}, including a humanized V_{HH} or camelized V_{H}. Non-limiting examples of (suitable combinations of) such framework sequences will become clear from the further disclosure herein.

Again, as generally described herein for the immunoglobulin sequences, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived).

However, it should be noted that the ISVD comprised in the multivalent ISVD polypeptide used in the present method is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVDs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

As described above, an ISVD may be a NANOBODY® ISVD or a suitable fragment thereof. For a general description of NANOBODY® ISVDs, reference is made to the further description below, as well as to the prior art cited herein. In this respect, it should however be noted that this description and the prior art mainly described NANOBODY® ISVDs of the so-called “V_{H}3 class” (i.e. ISVDS with a high degree of sequence homology to human germline sequences of the V_{H}3 class such as DP-47, DP-51 or DP-29). It should however be noted that the ISVD polypeptide used in the method described herein in its broadest sense can generally use any type of NANOBODY® ISVD, and for example also uses the NANOBODY® ISVDs belonging to the so-called “V_{H}4 class” (i.e. ISVDs with a high degree of sequence homology to human germline sequences of the V_{H}4 class such as DP-78), as for example described in WO 2007/118670.

Generally, NANOBODY® ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a NANOBODY® ISVD can be defined as an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a NANOBODY® ISVD can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
   one or more of the amino acid residues at positions 11, 37, 44, 45, 47, 83, 84, 103, 104 and 108 according to the Kabat numbering are chosen from the Hallmark residues mentioned in Table A below.

TABLE A
Hallmark Residues in NANOBODY ® ISVDs

| Position | Human V_{H}3 | Hallmark Residues |
|---|---|---|
| 11 | L, V; predominantly L | L, S, V, M, W, F, T, Q, E, A, R, G, K, Y, N, P, I; |
| | | preferably L |
| 37 | V, I, F; usually V | F⁽¹⁾, Y, V, L, A, H, S, I, W, C, N, G, D, T, P, preferably |
| | | F⁽¹⁾ or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or |
| | | Q⁽³⁾. |
| 45⁽⁸⁾ | L | L⁽²⁾, R⁽³⁾, P, H, F, G, Q, S, E, T, Y, C, I, D, V; preferably |
| | | L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, |
| | | N, D; preferably W⁽²⁾, L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; predominantly A | P⁽⁵⁾, S, H, L, A, V, I, T, F, D, R, Y, N, Q, G, E; preferably |
| | | P |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; predominantly | Q, L⁽⁷⁾, R, P, E, K, S, T, M, A, H; preferably Q or L⁽⁷⁾ |
| | L | |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

### 5.3 Multivalent ISVD Polypeptide and the Conformational Variant Thereof

Methods are provided for the purification or isolation of a multivalent ISVD polypeptide that comprises or consists of at least three or at least four ISVDs. The multivalent ISVD polypeptide to be isolated/purified by the methods described herein is obtainable by expression in a host. In particular, the multivalent ISVD polypeptide is obtainable by expression in a host that is not a CHO cell. The multivalent ISVD polypeptide is obtainable by expression in a lower eukaryotic host as described herein, such as e.g. in P. pastoris. Methods are provided for the production, purification, and isolation of a multivalent ISVD polypeptide that comprises or consists of at least three or at least four ISVDs. The multivalent ISVD polypeptide to be isolated/purified/produced by the methods can be produced in a host as described herein, such as a lower eukaryotic host. In one aspect, the multivalent ISVD polypeptide to be isolated/purified/produced by the methods can be produced in a yeast host as described herein, such as Pichia, e.g. in P. pastoris.

In general, the term “multivalent” indicates the presence of multiple ISVDs (binding units) in a polypeptide. In one embodiment, the polypeptide is at least “trivalent”, i.e., comprises or consists of at least three ISVDs. In another embodiment, the polypeptide is at least “tetravalent”, i.e. comprises or consists of at least four ISVDs. The polypeptide produced, purified and/or isolated in the method described herein can thus be “trivalent”, “tetravalent”, “pentavalent”, “hexavalent”, “heptavalent”, “octavalent”, “nonavalent”, etc., i.e., the polypeptide comprises or consists of three, four, five, six, seven, eight, nine, etc., ISVDs, respectively. In one embodiment the multivalent ISVD polypeptide is trivalent. In another embodiment the multivalent ISVD polypeptide is tetravalent. In still another embodiment, the multivalent ISVD polypeptide is pentavalent.

The multivalent ISVD construct comprising or consisting of at least three or at least four ISVDs can also be multispecific. The term “multispecific” refers to binding to multiple different target molecules. The multivalent ISVD construct can thus be “bispecific”, “trispecific”, “tetraspecific”, etc., i.e., can bind to two, three, four, etc., different target molecules, respectively.

For example, the polypeptide may be bispecific-trivalent, such as a polypeptide comprising or consisting of three ISVDs, wherein two ISVDs bind to human TNFα and one ISVD binds to human serum albumin (such as e.g. compound C, SEQ ID NO: 69). In another example, the polypeptide may be trispecific-tetravalent, such as a polypeptide comprising or consisting of four ISVDs, wherein one ISVD binds to human TNFα, two ISVDs bind to human IL23p19 and one ISVD binds to human serum albumin (such as e.g. compound B, SEQ ID NO: 2); or such as a polypeptide comprising or consisting of four ISVDs, wherein one ISVD binds to human TNFα, two ISVDs bind to human IL6 and one ISVD binds to human serum albumin (such as e.g. compound D, SEQ ID NO: 70; or compound E, SEQ ID NO:71). In still another example, the polypeptide may be trispecific-pentavalent, such as a polypeptide comprising or consisting of five ISVDs, wherein two ISVDs bind to human TNFα, two ISVDs bind to human OX40 L and one ISVD binds to human serum albumin (such as e.g. compound A; SEQ ID NO: 1).

The polypeptides consisting of at least three or at least four ISVDs to be produced/purified/isolated by the methods described herein can be linked by one or more suitable linkers, such as peptidic linkers. The use of linkers to connect two or more (poly)peptides is well known in the art. Exemplary peptidic linkers are shown in Table B. One often used class of peptidic linker are known as the “Gly-Ser” or “GS” linkers. These are linkers that essentially consist of glycine (G) and serine (S) residues, and usually comprise one or more repeats of a peptide motif such as the GGGGS (SEQ ID NO: 4) motif (for example, having the formula (Gly-Gly-Gly-Gly-Ser)ₙ in which n may be 1, 2, 3, 4, 5, 6, 7 or more). Some often-used examples of such GS linkers are 9GS linkers (GGGGSGGGS, SEQ ID NO: 7), 15GS linkers (n=3) and 35GS linkers (n=7). Reference is for example made to Chen et al., Adv. Drug Deliv. Rev. 2013 Oct. 15; 65(10): 1357-1369; and Klein et al., Protein Eng. Des. Sel. (2014) 27 (10): 325-330. In one embodiment, the polypeptide uses 9GS linkers to link the components of the polypeptide to each other. In one embodiment, the at least three or at least four ISVDs are connected to each other in a linear (i.e. non-branched) sequence, optionally via of one or more peptidic linkers.

The polypeptides consisting of at least three or at least four ISVDs to be produced/purified/isolated by the present methods may also comprise other groups, residues, moieties or binding units. These other groups, residues, moieties or binding units may provide the polypeptide with increased half-life, compared to the corresponding polypeptide without said one or more other groups, residues, moieties or binding units. For example, the binding unit can be an ISVD that binds to a serum protein, such as to a human serum protein such as human serum albumin (see e.g. WO 2012/175400, WO 2015/173325, WO 2017/080850, WO 2017/085172, WO 2018/104444, WO 2018/134234, WO 2018/134235). Further, the polypeptides consisting of at least three or at least four ISVDs to be produced/purified/isolated by the present methods may also comprise other suitable groups, residues, moieties or binding units necessary for any purification process (e.g., tags such as a His-tag).

The polypeptides comprising or consisting of at least three or at least four ISVDs to be produced/purified/isolated by the present methods may also form part of a protein or polypeptide, that e.g., comprises one or more further amino acid sequences (all optionally linked via one or more suitable linkers) that are not ISVDs but provide other functionalities. For example, and without limitation, the at least three or at least four ISVDs may be used as a binding unit in such a protein or polypeptide, which may optionally contain one or more further amino acid sequences which are not ISVDs that can serve as a binding unit (i.e., against one or more other targets) and/or as a functional unit.

TABLE B
Linker sequences (″ID″ refers to the SEQ ID NO as used herein)

| Name | ID | Amino acid sequence |
|---|---|---|
| 3A linker | 3 | AAA |
| 5GS linker | 4 | GGGGS |
| 7GS linker | 5 | SGGSGGS |
| 8GS linker | 6 | GGGGSGGS |
| 9GS linker | 7 | GGGGSGGGS |
| 10GS linker | 8 | GGGGSGGGGS |
| 15GS linker | 9 | GGGGSGGGGSGGGGS |
| 18GS linker | 10 | GGGGSGGGGSGGGGSGGS |
| 20GS linker | 11 | GGGGSGGGGSGGGGSGGGGS |
| 25GS linker | 12 | GGGGSGGGGSGGGGSGGGGSGGGGS |
| 30GS linker | 13 | GGGGSGGGGSGGGGSGGGGSGGGGSGGGGS |
| 35GS linker | 14 | GGGGSGGGGSGGGGSGGGGSGGGGSGGGGSGGGGS |
| 40GS linker | 15 | GGGGSGGGGSGGGGSGGGGSGGGGSGGGGSGGGGSGGGGS |
| G1 hinge | 16 | EPKSCDKTHTCPPCP |
| 9GS-G1 hinge | 17 | GGGGSGGGSEPKSCDKTHTCPPCP |
| Llama upper long | 18 | EPKTPKPQPAAA |
| hinge region | | |
| G3 hinge | 19 | ELKTPLGDTTHTCPRCPEPKSCDTPPPCPRCPEPKSCDTPPPCPRC |
| | | PEPKSCDTPPPCPRCP |

The multivalent ISVD polypeptide comprising or consisting of at least three or at least four ISVDs to be produced, purified, and/or isolated is the desired product of the production/purification/isolation method described herein. The term “(multivalent ISVD) polypeptide comprising or consisting of at least three or at least four ISVDs” in this regard is interchangeably used with “the polypeptide”, “the desired polypeptide (product)”, “the ISVD polypeptide”, “the desired ISVD polypeptide”, “the (multivalent) ISVD polypeptide (product)”, or “the (multivalent) ISVD construct” within this application. The desired polypeptide product is also referred to as “the product”, “the intact product”, or “the intact (ISVD) form”. The intact form appears as a main peak in analytical chromatographic techniques such as SE-HPLC and IEX-HPLC.

The “conformational variant” of the multivalent ISVD polypeptide comprising or consisting of at least three or at least four ISVDs is undesired and is to be converted into the desired ISVD polypeptide and/or to be removed from a composition comprising the intact product and the conformational variant by the method(s) described in the present application. The conformational variant is characterized by a more compact form compared to the intact product. The term “conformational variant” is thus interchangeably used with “variant”, “compact variant”, “compact conformational variant” or “compact form” within this application.

The compact variant is characterized by a decreased hydrodynamic volume compared to the desired polypeptide product. In general, the hydrodynamic volume is the apparent volume occupied by the expanded or swollen molecular coil along with the imbibed solvent. In other words, the hydrodynamic volume is how much space a particular polymer molecule takes up when it is in solution (effective hydrated volume of the macromolecule in solution). The hydrodynamic volume of a macromolecule can be deduced from its behavior in solution e.g., from its retention time in size-exclusion chromatography (SEC) and is thus a size-based dynamical property of a macromolecule. By measuring the hydrodynamic volume of a protein/polypeptide, SEC can assay protein tertiary structure (or even quaternary structures if suitable native conditions are used that preserve macromolecular interactions) allowing folded and unfolded versions or even folded and unfolded domains of the same protein/polypeptide to be distinguished (but not molecular weight). For example, the apparent hydrodynamic radius of a typical protein domain might be 14 Å and 36 Å for the folded and unfolded forms, respectively. SEC allows the separation of these two forms, as the folded form elutes much later due to its smaller size.

The compact variant is characterized by an altered surface charge and/or an altered hydrophobicity exposure (surface hydrophobicity) compared to the desired polypeptide product.

Without being bound by hypothesis—the compact conformation of the variant is due to intramolecular interaction between at least two of the at least three or at least four ISVD building blocks of the polypeptide (compared to the desired polypeptide product). Hence, the conformational variant can be characterized by at least two ISVDs interacting with each other resulting in a decreased hydrodynamic volume compared to the desired polypeptide product. Moreover, the compact variant can thus be characterized by at least two ISVDs interacting with each other resulting in an altered surface charge and/or an altered surface hydrophobicity compared to the desired polypeptide product.

Accordingly, the conformational variant can be distinguished from the desired polypeptide product by a shift in the hydrodynamic volume. Moreover, the conformational variant can be distinguished from the desired polypeptide product by a shift in surface charge and/or surface hydrophobicity. The conformational variant and the desired polypeptide product do not differ in their molecular weight. The conformational variant and the desired polypeptide product are thus not distinguishable by their molecular weight. Further, the conformational variant and the desired polypeptide product do not differ in their disulfide bridges. The conformational variant and the desired polypeptide product are thus not distinguishable by scrambled disulfide bridges.

Due to the alterations as described above, the conformational variant and the desired polypeptide product can be distinguished by an altered retention time of the conformational variant compared to the desired polypeptide product observed in analytical and/or preparative chromatographic techniques. For instance, the conformational variant can be distinguished from the desired polypeptide product by one or more analytical chromatographic techniques such as SE-HPLC and/or IEX-HPLC. In particular, the conformational variant can be distinguished from the desired polypeptide product by a shift in hydrodynamic volume, wherein said shift is indicated by an increased retention time in analytical SE-HPLC. Moreover, the conformational variant can be distinguished from the desired polypeptide product by a shift in surface charge, wherein said shift is indicated by an altered retention time in analytical IEX-HPLC. The increased retention time of the conformational variant compared to the intact product is identifiable by analytical SE-HPLC as a post peak shoulder or a resolved post peak in the chromatogram of said SE-HPLC. The alteration in surface charge of the conformational variant compared to the intact product is identifiable by analytical IEX-HPLC as a pre-peak shoulder or a resolved pre-peak, or as a post-peak shoulder or a resolved post-peak, respectively in the chromatogram of said IEX-HPLC. As apparent to the skilled person, whether the retention time of the conformational variant compared to the intact product is decreased or increased is dependent on both the quality and amount of difference in surface charge of the conformational variant compared to the intact product as well as the conditions used in the IEX-HPLC (e.g. resin, buffer, pH, salt concentration/ion strength, etc.). Accordingly, in one embodiment, the conformational variant is characterized by an increased retention time in IEX-HPLC. In another embodiment, the conformational variant is characterized by a decreased retention time in IEX-HPLC. As such, the conformational variant is characterized by an increased retention time in SE-HPLC compared to the intact product. The conformational variant is also characterized by an altered (decreased or increased) retention time in IEX-HPLC compared to the intact product.

Due to the above described alterations, the conformational variant can also be distinguished from the intact product by one or more preparative chromatographic techniques such as size exclusion chromatography (SEC), ion-exchange chromatography (IEX), e.g. cation-exchange chromatography (CEX), mixed-mode chromatography (MMC), and/or hydrophobic interaction chromatography (HIC). In particular, the conformational variant can be distinguished from the (desired) polypeptide by its presence in different fractions obtained from said preparative chromatographic techniques (due to an altered retention time of the conformational variant compared to the desired polypeptide product observed in said preparative chromatographic techniques). For instance, the conformational variant can be characterized by its presence in a side-fraction in preparative IEX (e.g., CEX), preparative MMC (e.g. based on a hydroxyapatite resin), and/or HIC (e.g. based on a HIC column resin or HIC membrane) compared to the desired polypeptide product eluting as the top fraction. As apparent to the skilled person, whether the conformational variant elutes as a pre-side fraction or a post-side fraction, i.e., whether the conformational variant elutes with a decreased or increased retention time, respectively, is dependent on both the quality and amount of difference in surface charge and/or surface hydrophobicity of the conformational variant compared to the desired polypeptide product as well as the conditions used in the respective preparative chromatographic technique used (e.g. resin, buffer, pH, salt concentration/ion strength, etc.).

Accordingly, after identification of the conformational variant by the specific analytical chromatographic techniques provided herein such as SE-HPLC and/or IEX-HPLC, the skilled person is able to adjust/optimize preparative chromatographic techniques to remove the conformational variant.

In a further aspect, the conformational variant can be distinguished from the desired polypeptide product by an alteration in potency, wherein the conformational variant has a decreased potency (as defined herein) compared to the desired polypeptide product.

Moreover, the conformational variant can be distinguished from the desired polypeptide product by its ability to be converted to the desired polypeptide product in a treatment method as described herein. More specifically, the conformational variant is characterized by its ability to be converted into the desired polypeptide product upon:

i) applying a low pH treatment in one or more steps of the isolation and/or purification process;
ii) applying a chaotropic agent in one or more steps of the isolation and/or purification process;
iii) applying a heat stress in one or more steps of the isolation and/or purification process; or
iv) a combination of any of i) to iii),
wherein the conversion is demonstrated by one or more analytical chromatographic techniques such as SE-HPLC and/or IEX-HPLC. In particular, the conversion is demonstrated by the decrease or (even) disappearance of the post-peak shoulder or the resolved post-peak in the chromatogram of analytical SE-HPLC. In addition, or in the alternative, the conversion is demonstrated by the decrease or (even) disappearance of the pre-peak shoulder or resolved pre-peak, or the post-peak shoulder or resolved post-peak in the chromatogram of analytical IEX-HPLC.

In addition, or in the alternative, the conversion is demonstrated by the partial or full recovery of the potency relative to the potency of the desired polypeptide product.

### 5.4 Production/Purification/Isolation Method

A method is provided for isolating or purifying the multivalent ISVD polypeptide product described above, wherein the multivalent ISVD polypeptide to be isolated or purified is obtainable by expression in a host. In one embodiment, the host is not a CHO cell. In one embodiment, the host is a lower eukaryotic host as provided herein (section 5.3 “Multivalent ISVD polypeptide and the conformational variant thereof”). The term “purify”, “purification”, or “purifying” as used herein means that the composition comprising the desired multivalent ISVD polypeptide product and the conformational variant is freed from impure elements (among which the conformational variant). The term “isolate”, “isolation”, or “isolating” as used herein means that the desired multivalent polypeptide product is set apart or separated from a composition comprising, in addition to impure elements, both the desired multivalent ISVD polypeptide product and the conformational variant thereof.

In addition, a method is provided for producing the multivalent ISVD polypeptide product in a host. In one embodiment, the host is not a CHO cell. In one embodiment, the host is a lower eukaryotic host as provided herein. The method may comprise transforming/transfecting the host cell or host organism with a nucleic acid encoding the polypeptide, expressing the polypeptide in the host, followed by one or more isolation and/or purification steps. Specifically, the method of producing a multivalent ISVD polypeptide product may comprise:

a) expressing, in a suitable host cell or host organism or in another suitable expression system, a nucleic acid sequence encoding the polypeptide; followed by:
b) isolating and/or purifying the desired polypeptide.

In a significant fraction of the multivalent ISVD polypeptides produced by the host such as lower eukaryotic host cells, the presence of a product related conformational variant is observed. The presence of this conformational variant might have an impact on the quality and the homogeneity of the final multivalent ISVD polypeptide product. A high product quality and homogeneity is, however, a prerequisite for e.g., the therapeutic use of these multivalent ISVD polypeptide products.

The present application describes methods for the production/purification/isolation of a composition comprising the multivalent ISVD polypeptide products with improved quality (i.e., with a reduced level of the conformational variant or its absence). The quality is improved by applying specified conditions in which (1) the conformational variant is converted into the desired polypeptide product and/or (2) the conformational variant is removed during an isolation or purification step of the multivalent ISVD polypeptide. Provided herein thus are methods of converting the product-related conformational variant into the ISVD-containing desired polypeptide product. Provided also are methods of removing the product-related conformational variant from a composition comprising the (desired) polypeptide product and the conformational variant thereof. Provided are methods of converting the product-related conformational variant into the (desired) ISVD polypeptide product and removing the product-related conformational variant from a composition comprising the (desired) ISVD polypeptide product and the conformational variant thereof.

### 5.4.1 Production of a Polypeptide Comprising or Consisting of at Least Three or at Least Four ISVDs

The present inventors have identified a conformational variant of a polypeptide comprising or consisting of at least three or at least four ISVDs upon production of the polypeptide in a host. The conformational variant was observed upon production in a host, in particular a host that is a lower eukaryote host as provided herein.

The skilled person is well aware of general methods for producing immunoglobulin single variable domains in host cells.

In a general embodiment, the method of producing a polypeptide that comprises at least three or at least four immunoglobulin single variable domains (ISVDs) comprises one or more purification/isolation steps that result in the conversion of the conformational variant into the desired ISVD polypeptide product and/or the removal of the conformational variant from a composition comprising the desired ISVD polypeptide product and the conformational variant thereof, as further detailed in sections 5.4.3 “Conversion of the conformational variant into the desired polypeptide product” and 5.4.4 “Removal of the conformational variant” below.

More particularly, the method for producing a polypeptide comprising at least three or at least four ISVDs at least comprises the following steps:
a) Optionally cultivating a host or host cell under conditions that are such that the host or host cell will multiply;
b) maintaining the host or host cell under conditions that are such that the host or host cell expresses and/or produces said polypeptide; and
c) isolating and/or purifying the secreted polypeptide from the medium, wherein said isolating and/or purifying comprises one or more purification/isolation steps that result in the conversion of the conformational variant into the desired ISVD polypeptide product and/or the removal of the conformational variant from a composition comprising the desired ISVD polypeptide product and the conformational variant thereof.

The ISVD polypeptide to be isolated/purified by the method described herein can be produced in a host. The host can be a host that is not a CHO cell. In particular, the host can be a lower eukaryotic host such as a yeast organism. Suitable yeast organisms for the production of the polypeptide to be isolated/purified are Pichia (Komagataella), Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis. In a particular embodiment the polypeptide to be purified/isolated is produced in Pichia, in particular in P. pastoris.

Production of ISVDs in lower eukaryotic hosts such as P. pastoris has been described by Frenken et al. 2000 (J. Biotechnol. 78: 11-21), WO 94/25591, WO 2010/125187, WO 2012/056000, WO 2012/152823 and WO2017/137579. The contents of these applications are explicitly referred to in the connection with general culturing techniques and methods, including suitable media and conditions. The skilled person can also devise suitable genetic constructs for expression of domains in host cells on the basis of common general knowledge.

The terms “host organism” and “host cell(s)” are jointly referred to herein as the “host”. In the production method described herein, any host (organism) or host cell can be used provided that they are suitable for the production of an ISVD containing polypeptide. In particular, hosts (such as lower eukaryotic hosts) are described wherein a portion of the polypeptides is produced in the form of a product-related conformational variant.

Specific examples of suitable hosts comprise prokaryotic organisms, such as coryneform bacteria or enterobacteriaceae. Also comprised are insect cells, in particular insect cells suitable for baculovirus mediated recombinant expression like Trioplusiani or Spodoptera frugiperda derived cells, including, but not limited to BTI-TN-5B1-4 High Five™ insect cells (Invitrogen), SF9 or Sf21 cells; mammalian cells such as CHO cells and lower eukaryotic hosts comprising yeasts such as Pichia (Komagataella), Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis. In one embodiment, yeast is used as the host, such as e.g. P. pastoris.

The host used in the production method will be capable of producing an ISVD containing polypeptide. It will typically be genetically modified to comprise one or more nucleic acid sequences encoding one or more ISVD containing polypeptides. Non-limiting examples of genetic modifications comprise the transformation e.g., with a plasmid or vector, or the transduction with a viral vector. Some hosts can be genetically modified by fusion techniques. Genetic modifications include the introduction of separate nucleic acid molecules into a host, e.g. plasmids or vectors, as well as direct modifications of the genetic material of the host, e.g. by integration into a chromosome of the host, e.g. by homologous recombination. Oftentimes a combination of both will occur, e.g. a host is transformed with a plasmid, which, upon homologous recombination will (at least partly) integrate into the host chromosome. The skilled person knows suitable methods of genetic modification of the host to enable the host to produce ISVD containing polypeptide.

Specific conditions and genetic constructs for the expression of nucleic acids and for the production of polypeptides are described in the art, for example the general culturing methods, plasmids, promoters and leader sequences described in WO 94/25591, Gasser et al. Biotechnol. Bioeng. 94: 535, 2006; Gasser et al. Appl. Environ. Microbiol. 73: 6499, 2007; or Damasceno et al. Microbiol. Biotechnol. 74: 381, 2007.

### 5.4.2 Purification of a Polypeptide Comprising or Consisting of at Least Three or at Least Four ISVDs

The skilled person is well aware of general methods for purifying ISVD polypeptides (such as V_{H}s and V_{HH}s).

For example, the purification of ISVDs has been described in WO 2010/125187 and WO 2012/056000.

After the production/expression of the polypeptide, the host can be removed from the culture medium by routine means. For example, the host can be removed by centrifugation or filtration. The solution obtained by removal of the host from the culture medium is also referred to as culture supernatant or clarified culture supernatant.

The multivalent ISVD product can be purified from culture supernatant by standard methods. Standard methods include, but are not limited to chromatographic methods, including size exclusion chromatography (SEC), ion exchange chromatography (IEX), affinity chromatography (AC), hydrophobic interaction chromatography (HIC), mixed-mode chromatography (MMC). These methods can be performed alone or in combination with other purification methods, e.g., precipitation. The skilled person can devise suitable combinations of purification methods for ISVDs and ISVD containing polypeptides on the basis of common general knowledge. For specific examples the art cited herein is referred to.

It is envisaged that any of the conditions or a combination thereof, that convert or remove the conformational variant as described in detail below (sections 5.4.3 “Conversion of the conformational variant into the desired polypeptide product” and 5.4.4 “Removal of the conformational variant”), can be applied before, at or between, or after any step of these purification methods.

Any or all chromatographic steps can be carried out by any mechanical means. Chromatography may be carried out, for example, in a column. The column may be run with or without pressure and from top to bottom or bottom to top. The direction of the flow of fluid in the column may be reversed during the chromatography process. Chromatography may also be carried out using a batch process in which the solid media is separated from the liquid used to load, wash, and elute the sample by any suitable means, including gravity, centrifugation, or filtration.

Chromatography may also be carried out by contacting the sample with a filter that absorbs or retains some molecules in the sample more strongly than others. In the following description, the various embodiments are mostly described in the context of chromatography carried out in a column. It is understood, however, that use of a column is merely one of several chromatographic modalities that may be used, and the illustration using a column does not limit the application to column chromatography, as those skilled in the art may readily apply the teachings to other modalities as well, such as those using a batch process or filter.

Suitable supports may be any currently available or later developed materials having the characteristics necessary to practice the claimed method, and may be based on any synthetic, organic, or natural polymer. For example, commonly used support substances include organic materials such as cellulose, polystyrene, agarose, sepharose, polyacrylamide polymethacrylate, dextran and starch, and inorganic materials, such as charcoal, silica (glass beads or sand) and ceramic materials. Suitable solid supports are disclosed, for example, in Zaborsky “Immobilized Enzymes” CRC Press, 1973, Table IV on pages 28-46.

General method conditions, solutions and/or buffers, as well as their concentration ranges for use in the different chromatographic processes can be determined by one skilled in the art of chromatography, based on standard handbooks on chromatography (see e.g. Gunter Jagschies, Eva Lindskog (ed.) Biopharmaceutical Processing, Development, Design, and Implementation of Manufacturing Processes, 1st Ed. 2017, Elsevier).

The first step of an ISVD polypeptide purification process is often referred to as “the capture step”. The purpose of the capture step is to have a first reduction of process-related impurities (for example, but not limited to, host cell proteins (HCPs), color and DNA) and to capture the ISVD polypeptide product while maintaining a high recovery. In one embodiment, the capture step refers to the first purification step on protein A chromatography in bind and elute mode.

The second step of a purification process is often referred to as “the polish step” which aims at purity improvement. For instance, as the second purification step of an ISVD polypeptide purification process an ion exchange chromatography step in bind and elute mode can be used to remove/reduce product related variants (e.g., but not limited to, High-molecular Weight (HMW) species, Low-Molecular Weight (LMW) species, and other charged variants) as well as some process related impurities (e.g., but not limited to, HCP, residual Protein A, DNA) still present after the capture step.

In one exemplary embodiment, the multivalent ISVD polypeptide can be purified from culture supernatant by a combination of affinity chromatography on Protein A, ion exchange chromatography and size exclusion chromatography. Reference to any “step of purification”, includes, but is not limited to these particular methods.

### Protein A—Based Chromatography

In one embodiment, the ISVD polypeptide containing preparations may be purified by Protein A chromatography. Staphylococcal Protein A (SpA) is a 42 kDa protein composed of five nearly homologous domains named as E, D, A, B and C in order from the N-terminus (Sjodhal Eur. J. Biochem. 78: 471-490 (1977); Uhlen et al. J. Biol. Chem. 259: 1695-1702 (1984)). These domains contain approximately 58 residues, each sharing about 65%-90% amino acid sequence identity. Binding studies between Protein A and antibodies have shown that while all five domains of SpA (E, D, A, B and C) bind to an IgG via its Fc region, domains D and E exhibit significant Fab binding (Ljungberg et al. Mol. Immunol. 30(14): 1279-1285 (1993); Roben et al. J. Immunol. 154: 6437-6445 (1995); Starovasnik et al. Protein Sei. 8: 1423-1431 (1999). The Z-domain, a functional analogue and energy-minimized version of the B domain (Nilsson et al. Protein Eng. 1: 107-113 (1987)), was shown to have negligible binding to the antibody variable domain region (Cedergren et al. Protein Eng. 6(4): 441-448 (1993); Ljungberg et al. (1993) supra; Starovasnik et al. (1999) supra).

Until recently, commercially available Protein A stationary phases employed SpA (isolated from Staphylococcus aureus or expressed recombinantly) as their immobilized ligand. Using these columns, it has not been possible to use alkaline conditions for column regeneration and sanitation as is typically done with other modes of chromatography using non-proteinaceous ligands (Ghose et al. Biotechnology and Bioengineering Yol. 92 (6): 665-73 (2005)). A new resin (MabSELECT™ SuRe) has been developed to withstand stronger alkaline conditions (Ghose et al. (2005) supra). Using protein engineering techniques, a number of asparagine residues were replaced in the Z-domain of protein A and a new ligand was created as a tetramer of four identically modified Z-domains (Ghose et al. (2005) supra).

Accordingly, purification methods can be carried out using commercially available Protein A columns according to manufacturers' specification. For instance, MabSELECT™ columns or MabSELECT™ SuRe columns (GE Healthcare Products) can be used. MabSELECT™ is a commercially available resin containing recombinant SpA as its immobilized ligand. Other commercially available sources of Protein A column including, but not limited to, PROSEP-ATM (Millipore, U.K.), which consists of Protein A covalently coupled to controlled pore glass, can be usefully employed. Other useful Protein A formulations include Protein A Sepharose FAST FLOW™ (Amersham Biosciences, Piscataway, N.J.), Amsphere™ A3 (JSR Life Sciences), and TOYOPEARL™ 650M Protein A (TosoHaas Co., Philadelphia, Pa.).

Protein purification by Protein A-based chromatography may be performed in a column containing an immobilized Protein A ligand (typically a column packed with modified support of methacrylate copolymer or agarose beads to which is affixed an adsorbent consisting of Protein A or functional derivatives thereof). The column is typically equilibrated with a buffer and a sample containing a mixture of proteins (the target protein, plus contaminating proteins) is loaded onto the column. As the mixture passes through the column, the target protein binds to the adsorbent (Protein A or derivative thereof) within the column, while some unbound impurities and contaminants flow through. Bound protein is then eluted from the column. In this process the target protein is bound to the column while impurities and contaminants flow through. Target protein is subsequently recovered from the eluate.

In a general embodiment, methods are provided of purifying/isolating a polypeptide that comprises at least three or at least four immunoglobulin single variable domains (ISVDs), wherein the methods comprise one or more purification/isolation steps that result in the conversion of the conformational variant into the desired ISVD polypeptide product and/or the removal of the conformational variant from a composition comprising the desired ISVD polypeptide product and the conformational variant thereof, as further detailed in sections 5.4.3 “Conversion of the conformational variant into the desired polypeptide product” and 5.4.4 “Removal of the conformational variant”.

5.4.3 Conversion of the Conformational Variant into the Desired Polypeptide Product

In one aspect, the composition comprising the polypeptide product and a conformational variant thereof is purified by applying conditions that convert the conformational variant into the desired polypeptide product.

In this aspect, the conditions that convert the conformational variant into the desired polypeptide product can be selected from a) applying a low pH treatment, b) applying a chaotropic agent c) applying a heat stress, and d) applying a combination of any of the treatments of a) to c). For instance, in one embodiment, the conformational variant is converted into the desired polypeptide product by applying a low pH treatment and a chaotropic agent. In another embodiment, the conformational variant is converted into the desired polypeptide product by applying a low pH treatment and a heat treatment. In a further embodiment, the conformational variant is converted into the desired polypeptide product by applying heat stress and a chaotropic agent. In still another embodiment, the conformational variant is converted into the desired polypeptide product by applying a low pH treatment, a chaotropic agent, and heat stress.

The conditions that convert the conformational variant into the desired polypeptide product may be applied (without being limiting) on culture supernatant comprising the multivalent ISVD polypeptide (before the capture step), during the capture step, after the capture step but before the polish step, during the polish step, or after the polish step. The conditions that convert the conformational variant into the desired polypeptide product may be applied on a partially or highly purified preparation of the multivalent ISVD polypeptide. The conditions that convert the conformational variant into the desired polypeptide product may be also applied on a column on a clarified supernatant, or a partially or highly purified preparation of the ISVD containing polypeptide. The conditions that convert the conformational variant into the desired polypeptide product can also be applied during another step, such as before or after a filtering step or any other step in the purification.

In the following, the conditions that convert the conformational variant into the desired polypeptide product are discussed in more detail. Applying these conditions will also be referred to as “treatment” of the multivalent ISVD polypeptide.

### Low pH Treatment

The conformational variant can be converted into the desired polypeptide product by a low pH treatment.

The low pH treatment can be applied anytime during the purification/isolation process of the multivalent ISVD polypeptide. In one embodiment, the low pH treatment is applied before a purification step based on a chromatographic technique. In another embodiment, the low pH treatment is applied during a purification step based on a chromatographic technique, e.g. a Protein A-based affinity chromatography (AC). For instance, the low pH treatment can be applied during a Protein A-based affinity chromatography ISVD polypeptide capture step. In another embodiment, the low pH treatment is applied after a purification step based on a chromatographic technique. For instance, the low pH treatment can be applied after a Protein A-based affinity chromatography ISVD polypeptide capture step (and before a ISVD polypeptide polish step). In the alternative, the low pH treatment can be applied after an ISVD polypeptide polish step.

The low pH treatment comprises decreasing the pH of a composition comprising the desired polypeptide product and the conformational variant thereof to about pH 3.2 or less for a sufficient amount of time such that the conformational variant is converted into the intact ISVD polypeptide product.

The low pH treatment comprises decreasing the pH of a composition comprising the desired polypeptide product and the conformational variant thereof to about pH 3.0 or less for a sufficient amount of time such that the conformational variant is converted into the intact ISVD polypeptide product.

The low pH treatment thus comprises decreasing the pH of a composition comprising the intact polypeptide product and the conformational variant thereof (e.g. the capture eluate after a (protein A) capture step) to about pH 3.2 or less, to about pH 3.1 or less, to about pH 3.0 or less, to about pH 2.9 or less, to about pH 2.8 or less, to about pH 2.7 or less, to about pH 2.6 or less, to about pH 2.5 or less, to about pH 2.4 or less, to about pH 2.3 or less, to about pH 2.2 or less, to about pH 2.1 or even less. Specifically, the pH of the composition can be decreased to about pH 2.9, to about pH 2.8, to about pH 2.7, to about pH 2.6, to about pH 2.5, to about pH 2.4, to about pH 2.3, to about pH 2.2, or to about 2.1. In one embodiment, the pH is decreased to between about pH 3.2 and about pH 2.1, to between about pH3.0 and about pH 2.1, to between about pH 2.9 and about pH 2.1, to between about pH 2.7 and about pH 2.1. In another embodiment, the pH is decreased to between about pH 2.6 and about pH 2.3. In another embodiment, the pH is decreased to between about pH 2.5 and about pH 2.1.

In the low pH treatment, the pH can be decreased by any routine means. For example, the pH of the composition comprising the desired polypeptide product and the conformational variant thereof can be decreased using HCl (e.g., in a stock concentration of 0.1M-3M, such as 0.1 M, 1 M, 3M, or 2.7M) or using Glycine (e.g. in a stock concentration of 0.1M). The skilled person can readily choose other suitable means.

In one embodiment, the low pH treatment is applied during a purification step based on a chromatographic technique, e.g. a Protein A-based affinity chromatography. The elution buffer used for the Protein A-based affinity chromatography may have a pH of equal to or less than about pH 2.5. Alternatively, the elution buffer used for the Protein A-based affinity chromatography has a pH such that the resulting eluate containing the polypeptide has a pH of equal to or less than about pH 3.2, such as less than about pH 2.9. Subsequent elution of the polypeptide from the Protein A column using an elution buffer as indicated above, the pH of the resulting eluate containing the polypeptide can (optionally) be additionally decreased to a pH of equal to or less than pH 2.5. In another embodiment, the pH of the resulting eluate can be adjusted to a pH of equal to or less than about pH 3.2 for at least about 0.5 hours, such as 1 hour or 2 hours. In another embodiment, the pH of the resulting eluate can be adjusted to a pH of equal to or less than about pH 2.9 for at least about 0.5 hours, such as 1 hour or 2 hours. In still another embodiment, the pH of the resulting eluate can be adjusted to a pH of equal to or less than about pH 2.7 for at least about 1 hour. In another embodiment, the chromatographic technique is a Protein A-based affinity chromatography, wherein the elution buffer has a pH of about pH 2.2, and wherein the pH of the resulting eluate is adjusted to a pH of about pH 2.5 for at least about 1.5 hour.

The present technology also provides methods of identifying a conformational variant of a polypeptide comprising or consisting of at least three or at least four ISVDs by analytical chromatographic methods such as SE-HPLC and IEX-HPLC. The present technology further provides for the concept of converting the conformational variant into the intact product by low pH treatment. Hence, based on the concept provided herein the skilled person is able to adjust the low pH treatment described herein to any polypeptide comprising or consisting of at least three or at least four ISVDs in terms of both optimal acidic pH as well as incubation time.

The low pH treatment can be terminated by increasing the pH of the composition comprising the polypeptide. The low pH treatment can be terminated by increasing the pH of the low pH treated composition by at least one pH unit. For instance, if the low pH treatment was performed at about pH 2.7, the treatment can be terminated by increasing the pH to at least about pH 3.7. The low pH treatment can be terminated by increasing the pH of the low pH treated composition by at least two pH units. For instance, if the low pH treatment was performed at about pH 2.7, the treatment can be terminated by increasing the pH to at least about pH 4.7. Accordingly, the low pH treatment can be terminated by increasing the pH to about pH 3.5 or more, to about pH 4.0 or more, to about pH 4.5 or more, to about pH 5.0 or more, to about pH 5.5 or more, to about pH 6.0 or more, to about pH 6.5 or more, to about pH 7.0 or more, to about pH 7.5 or more, to about pH 8.0 or more, etc. However, increasing the pH too high (e.g. to about pH 9 or higher) may result in (severe) degradation of the polypeptide product. As such, low pH treatment is terminated by increasing the pH to a pH between about pH 4 and about pH 8, or between about pH 5 and about pH 7.5. As apparent to the skilled person, the pH increase can be adapted to the pH required for possible subsequent purification, formulation or storage steps. In the present application, termination of the low pH treatment is used interchangeably with “pH neutralization”.

For terminating the low pH treatment, the pH can be increased by any routine means. Without being limiting, for example, the pH of the composition can be increased using NaOH (e.g., in a stock concentration of 0.1 M or 1 M) or using sodium acetate (e.g., in a stock concentration of 1 M). The skilled person can readily choose other suitable means.

Based on the methods described herein, the skilled person is able to determine the time that is necessary to convert the conformational variant into the desired polypeptide product. For instance, the low pH treatment is applied for a sufficient amount of time, up to when the conformational variant is essentially no longer detectable by a chromatographic technique described herein. For instance, the low pH treatment is applied for a sufficient amount of time, up to when essentially no post peak shoulder or resolved post peak (indicating the conformational variant) is observed in the chromatogram of the composition post low pH treatment using analytical SE-HPLC. In addition, or in the alternative, the low pH treatment is applied for a sufficient amount of time, up to when essentially no pre/post peak shoulder or resolved pre/post (indicating the conformational variant) is observed in the chromatogram of the composition post low pH treatment using analytical IEX-HPLC. In this regard, the low pH treatment can be applied for at least about 0.5 hours, for at least about 1 hour, for at least about 1.5 hours, for at least about 2 hours, for at least about 2.5 hours, for at least about 3 hours, for at least about 3.5 hours, for at least about 4 hours, for at least about 6 hours, for at least about 8 hours, for at least about 12 hours, for at least about 24 hours. For instance, the low pH treatment can be applied for about 0.5 hours, about 1 hour, about 1.5 hours, about 2 hours, about 2.5 hours, about 3 hours, about 3.5 hours, about 4 hours, about 6 hours, about 8 hours, about 12 hours, about 24 hours. In a specific embodiment, the low pH treatment can be applied for at least about 1 hour, or at least about two hours, or for at least about 4 hours.

In an embodiment, the pH is decreased to between about pH 3.2 and about 2.1 for at least 0.5 hours, to between about pH 2.9 and about 2.1 for at least 0.5 hours, to between about pH 2.7 and about 2.1 for at least 0.5 hours, e.g. to about pH 2.9, to about pH 2.7, to about pH 2.5, or to about pH 2.3 for 0.5 hours. In another embodiment, the pH is decreased to between about pH 3.2 and about 2.1 for at least 1 hour, to between about pH 2.9 and about 2.1 for at least 1 hour, to between about pH 2.7 and about 2.1 for at least 1 hour, e.g. to about pH 2.9, to about pH 2.7, to about pH 2.5, or to about pH 2.3 for 1 hour. In still another embodiment, the pH is decreased to between about pH 3.2 and about 2.1 for at least 2 hours, to between about pH 2.9 and about 2.1 for at least 2 hours, to between about pH 2.7 and about 2.1 for at least 2 hours, e.g. to about pH 2.9, to about pH 2.7, to about pH 2.5, or to about pH 2.3 for 2 hours. In still another embodiment, the pH is decreased to between about pH 3.2 and about 2.1 for at least 4 hours, to between about pH 2.9 and about 2.1 for at least 4 hours, to between about pH 2.7 and about 2.1 for at least 4 hours, e.g. to about pH 2.9, to about pH 2.7, to about pH 2.5, or to about pH 2.3 for 4 hours. In another embodiment, the pH is decreased to between about pH 2.6 and about pH 2.3 for at least 1 hour, or for at least 2 hours, e.g. to about pH 2.6 for 1 or 2 hours. In another embodiment, the pH is decreased to between about pH 2.5 and about pH 2.1 for at least 1 hour, or for at least 2 hours, e.g. to about pH 2.4 or pH 2.5 for 2 hours.

The low pH treatment can be applied at a wide range of temperatures with the proviso that the temperature does not result in the irreversible denaturation or degradation of the ISVD polypeptide. Examples include, but are not limited to temperatures between about 4° C. and about 30° C. Accordingly, the low pH treatment can be applied at about 30°, 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4° C. The skilled person can readily choose a suitable temperature for the low pH treatment. In an embodiment, the low pH treatment is applied at a temperature between about 15° C. and about 30° C. In another embodiment, the low pH treatment is applied at a temperature between about 4° C. and about 12° C. In another embodiment, the low pH treatment is applied at room temperature (RT), i.e., at between about 20° C. and 25° C.

### Chaotropic Agent Treatment

The conformational variant can also be converted into the desired polypeptide product by applying a chaotropic agent.

A chaotropic agent in general interferes with intermolecular and intramolecular interactions mediated by non-covalent forces such as hydrogen bonds, van der Waals forces and hydrophobic interactions, thereby increasing the entropy of the system. With respect to biomolecules, chaotropic agents are able to disrupt the structure of, and denature, macromolecules such as proteins and nucleic acids (e.g., DNA and RNA). Chaotropic agents are well known to the skilled person and comprise (without being limited to) n-butanol, ethanol, guanidinium chloride (GuHCl), lithium perchlorate, lithium acetate, magnesium chloride, phenol, 2-propanol, sodium dodecyl sulfate, thiourea, and urea. In one embodiment, the conformational variant is converted into the desired polypeptide product by applying a chaotropic agent which is GuHCl or urea. In specific embodiment, the conformational variant is converted into the desired polypeptide product by applying a chaotropic agent which is GuHCl.

The chaotropic agent can be applied anytime during the purification/isolation process of multivalent ISVD polypeptide. In one embodiment, the chaotropic agent is applied before a purification step based on a chromatographic technique (e.g., before the ISVD polypeptide capture step or before an ISVD polypeptide polish step). In another embodiment, the chaotropic agent is applied after a purification step based on a chromatographic technique (e.g., after the ISVD polypeptide capture step or after an ISVD polypeptide polish step). In another embodiment, the chaotropic agent is applied directly following a purification step based on a chromatographic technique, wherein the chromatographic technique is a Protein A-based affinity chromatography (e.g. used for ISVD polypeptide capture step). Thus, in one embodiment, the chaotropic agent is applied directly after the Protein A-based ISVD polypeptide capture step and before any polish steps. In another embodiment, the chaotropic agent is applied directly after an ISVD polypeptide polish step.

The skilled person is well aware that the chaotropic agent has to be applied in a concentration that enables conversion of the conformational variant into the desired polypeptide product but does not result in its irreversible denaturation or degradation. Based on the methods described herein, the skilled person is able to determine which concentration of the chaotropic agent is suitable for converting the conformational variant into the desired polypeptide product. A suitable concentration is applied, when the conformational variant is essentially no longer detectable by a chromatographic technique described herein. For instance, a suitable concentration is applied, when essentially no post peak shoulder or resolved post peak (indicating the conformational variant) is observed in the chromatogram of the composition post chaotropic agent treatment using analytical SE-HPLC. In addition, or in the alternative, a suitable concentration is applied, when essentially no pre/post peak shoulder or resolved pre/post peak (indicating the conformational variant) is observed in the chromatogram of the composition post chaotropic agent treatment using analytical IEX-HPLC. Irreversible denaturation or degradation of the ISVD polypeptide product by the chaotropic agent can be excluded if the respective SE-HPLC or IEX-HPLC chromatograms do not show formation of high-molecular-weight species (HMW species) (pre-peak in SE-HPLC) and/or a decrease of the total area (loss of product) or a decrease of the main peak in IEX-HPLC and/or SE-HPLC.

In one aspect, the chaotropic agent is GuHCl in a final concentration of between about 0.5 molar (M) and about 3 M, between about 0.5 M and about 2.5 M, between about 1 M and about 2.5 M, between about 1 M and about 2 M, such as about 1 M, about 2 M, about 2.5 M or about 3 M. In another aspect, the chaotropic agent is GuHCl in a final concentration of at least about 1 M, or at least about 2 M.

Based on the methods described herein, the skilled person is able to determine the time that is necessary to convert the conformational variant into the desired polypeptide product. For instance, the chaotropic agent treatment is applied for a sufficient amount of time, up to when the conformational variant is essentially no longer detectable by a chromatographic technique described herein. For instance, the chaotropic agent treatment is applied for a sufficient amount of time, up to when essentially no post peak shoulder or resolved post peak (indicating the conformational variant) is observed in the chromatogram of the composition post chaotropic agent treatment using analytical SE-HPLC. In addition, or in the alternative, the chaotropic agent treatment is applied for a sufficient amount of time, up to when essentially no pre/post peak shoulder or resolved pre/post peak (indicating the conformational variant) is observed in the chromatogram of the composition post chaotropic agent treatment using analytical IEX-HPLC. The skilled person is well aware that the chaotropic agent has to be applied for a time that enables conversion of the conformational variant into the desired polypeptide product but does not result in its irreversible denaturation or degradation. Irreversible denaturation or degradation of the ISVD polypeptide product by the chaotropic agent can be excluded if the respective SE-HPLC or IEX-HPLC chromatograms do not show the formation of high-molecular-weight species (HMW species) (pre-peak in SE-HPLC) and/or a decrease of the total area (loss of product) or a decrease of the main peak in IEX-HPLC and/or SE-HPLC. In this regard, the chaotropic agent treatment can be applied for at least about 0.5 hours, for at least about 1 hour, for at least about 1.5 hours, for at least about 2 hours, for at least about 2.5 hours, for at least about 3 hours, for at least about 3.5 hours, for at least about 4 hours, for at least about 6 hours, for at least about 8 hours, for at least about 12 hours. For instance, the chaotropic agent can be applied for about 0.5 hours, about 1 hour, about 1.5 hours, about 2 hours, about 2.5 hours, about 3 hours, about 3.5 hours, about 4 hours, about 6 hours, about 8 hours, about 12 hours. In one embodiment, the chaotropic agent can be applied for at least about 0.5 hours, or for at least about 1 hour.

In this aspect, GuHCl is applied for at least about 0.5 hours, or for at least about 1 hour. In one embodiment, the chaotropic agent is GuHCl in a final concentration of between about 1 M and about 2M for about 0.5 hours. In another embodiment, the chaotropic agent is GuHCl in a final concentration of between about 1 M and about 2M for about 1 hour.

The present technology provides for methods of identifying a conformational variant of a polypeptide comprising or consisting of at least three or at least four ISVDs by analytical chromatographic methods such as SE-HPLC and IEX-HPLC. The present technology further provides for the concept of converting the conformational variant into the intact product by treatment with a chaotropic agent. Hence, based on the concept provided herein the skilled person is able to adjust the chaotropic agent treatment described herein to any polypeptide comprising or consisting of at least three or at least four ISVDs in terms of both chaotropic agent concentration as well as incubation time.

The chaotropic agent treatment can be terminated by transferring the ISVD polypeptide product to a new buffer system (without chaotropic agent). The transfer can be accomplished by routine means e.g., dialysis, diafiltration or a chromatographic method (e.g., size exclusion or buffer exchange chromatography). For example, the ISVD polypeptide product can be transferred into PBS by dialysis. The ISVD polypeptide product may also be transferred into physiological saline. The skilled person can readily choose other suitable buffer systems. The buffer choice may depend on buffer conditions required for a potential subsequent purification, formulation or storage steps.

The chaotropic agent treatment can be applied at a wide range of temperatures with the proviso that the temperature does not result in the irreversible denaturation or degradation of the ISVD polypeptide. Examples include, but are not limited to temperatures between about 4° C. and about 30° C. Accordingly, the chaotropic agent treatment can be applied at about 30°, 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4° C. The skilled person can readily choose a suitable temperature for the chaotropic agent treatment. In an embodiment, the chaotropic agent treatment is applied at a temperature between about 15° C. and about 30° C. In another embodiment, the chaotropic agent treatment is applied at a temperature between about 4° C. and about 12° C. In another embodiment, the chaotropic agent treatment is applied at room temperature, i.e., at between about 20° C. and 25° C.

### Heat Treatment

The conformational variant can also be converted into the desired polypeptide product by applying a heat stress. The terms “heat treatment” and “heat stress” are used interchangeably herein.

The heat stress can be applied anytime during the purification/isolation process of the multivalent ISVD polypeptide. In one embodiment, the heat stress is applied before a purification step based on a chromatographic technique. In another embodiment, the heat stress is applied after a purification step based on a chromatographic technique. For instance, the heat stress can be applied after a Protein A-based affinity chromatography ISVD polypeptide capture step (and before an ISVD polypeptide polish step). In the alternative, the heat stress can be applied after any ISVD polypeptide polish step.

The heat stress is applied at a suitable temperature between 40° C. and 60° C. that enables conversion of the conformational variant into the desired polypeptide product, but that does not result in its irreversible denaturation or degradation. Based on the methods described herein, the skilled person is able to determine which temperature is suitable for converting the conformational variant into the desired polypeptide product. A suitable temperature is applied, when the conformational variant is essentially no longer detectable by a chromatographic technique described herein. For instance, a suitable temperature is applied, when essentially no post peak shoulder or resolved post peak (indicating the conformational variant) is observed in the chromatogram of the composition post heat stress using analytical SE-HPLC. In addition, or in the alternative, a suitable temperature is applied, when essentially no pre/post peak shoulder or resolved pre/post peak (indicating the conformational variant) is observed in the chromatogram of the composition post heat stress using analytical IEX-HPLC. Irreversible denaturation or degradation of the ISVD polypeptide product by heat stress can be excluded if the respective SE-HPLC or IEX-HPLC chromatograms do not show formation of high-molecular-weight species (HMW species) (pre-peak in SE-HPLC) and/or a decrease of the total area (loss of product) or a decrease of the main peak in IEX-HPLC and SE-HPLC. Accordingly, the heat stress applied to convert the conformational variant into the desired polypeptide product comprises incubating the composition at about 40° C. to about 60° C., at about 45° C. to about 60° C., or at about 50° C. to about 60° C. The heat stress can also comprises incubating the composition at about 40° C. to about 55° C., at about 45° C. to 55° C., or at about 48° C. to about 52° C., such as at about 50° C.

Based on the methods described herein, the skilled person is able to determine the time that is necessary to convert the conformational variant into the desired polypeptide product. The heat stress is applied for a sufficient amount of time, up to when the conformational variant is essentially no longer detectable by a chromatographic technique described herein. For instance, the heat stress is applied for a sufficient amount of time, up to when essentially no post peak shoulder or resolved post peak (indicating the conformational variant) is observed in the chromatogram of the composition post heat stress using analytical SE-HPLC. In addition, or in the alternative, the heat stress is applied for a sufficient amount of time, up to when essentially no pre/post peak shoulder or resolved pre/post peak (indicating the conformational variant) is observed in the chromatogram of the composition post heat stress using analytical IEX-HPLC. The skilled person is well aware that the heat stress has to be applied for a time that enables conversion of the conformational variant into the desired polypeptide product, but that does not result in its irreversible denaturation or degradation. Irreversible denaturation or degradation of the ISVD polypeptide product by heat stress can be excluded if the respective SE-HPLC or IEX-HPLC chromatograms do not show formation of high-molecular-weight species (HMW species) (pre-peak in SE-HPLC) or a decrease of the total area (loss of product) or a decrease of the main peak in IEX-HPLC and SE-HPLC. In this regard, the heat stress shall be applied no longer than 4 hours. Thus, the heat stress can be applied for at least about 0.5 hours, for at least about 1 hour, for at least about 1.5 hours, for at least about 2 hours, for at least about 2.5 hours, for at least about 3 hours, for at least about 3.5 hours, about 4 hours, but not longer than 4 hours. In particular, the heat stress can be applied for about 0.5 hours, about 1 hour, about 1.5 hours, about 2 hours, about 2.5 hours, about 3 hours, about 3.5 hours, about 4 hours. In an embodiment, the heat stress is applied for at least about 0.5 hours, or for at least about 1 hour, e.g. at 50° C. for about 1 hour. In another embodiment, the heat stress is applied for about 4 hours, e.g. at 50° C. for about 4 hours.

The present technology provides for methods of identifying a conformational variant of a polypeptide comprising or consisting of at least three or at least four ISVDs by analytical chromatographic methods such as SE-HPLC and IEX-HPLC. The present technology further provides for the concept of converting the conformational variant into the intact product by heat treatment. Hence, based on the concept provided herein the skilled person is able to adjust the heat treatment to any polypeptide comprising or consisting of at least three or at least four ISVDs in terms of both optimal heat stress temperature as well as incubation time.

The heat stress can be terminated by adjusting the composition comprising the ISVD polypeptide product to a temperature below about 30° C., i.e., to any temperature between about 4° C. and about 30° C. Accordingly, the heat treatment is terminated by adjusting the temperature of the composition to about 30°, 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4° C. In an embodiment, the heat treatment is terminated by adjusting the temperature of the composition to between about 15° C. and about 30° C. In another embodiment, the heat treatment is terminated by adjusting the temperature of the composition to between about 4° C. and about 12° C. In another embodiment, the heat treatment is terminated by adjusting the temperature of the composition to room temperature, i.e., to between about 20° C. and about 25° C. The temperature adjustment (for termination of the heat treatment) can be adapted to a temperature required for a potential subsequent purification, formulation or storage steps.

General Aspect Regarding the Conditions that Convert the Conformational Variant into the Desired Polypeptide Product

The above described treatment conditions to convert the conformational variant into the desired polypeptide product can be applied using a wide range of buffers suitable for protein purification/formulation, in particular any known buffer suitable for antibody purification/formulation. Examples include, but are not limited to PBS, phosphate buffer, acetate, histidine buffer, Tris-HCl, glycine buffers. The ISVD polypeptide may also be present in physiological saline. The skilled person can readily choose other suitable buffer systems.

Any of the above described condition that convert the conformational variant into the desired polypeptide product, or any combination thereof can be combined with any method that removes the conformational variant as further described below.

Based on the concept of low pH treatment, treatment with a chaotropic agent and heat treatment provided herein, the skilled person is able to adjust the treatment conditions of described herein to any polypeptide comprising or consisting of at least three or at least four ISVDs in terms of optimal pH, chaotropic agent concentration, and/or heat stress temperature as well as incubation time.

### 5.4.4 Removal or Reduction of the Conformational Variant

Removal or reduction means that the product-related conformational variant is physically separated from a composition comprising both the desired ISVD polypeptide product and the product-related conformational variant. The correct meaning will be apparent from the context. In the prior art the skilled person had no knowledge about the existence of a conformational variant of a polypeptide comprising or consisting of at least three or at least four ISVDs when produced in lower eukaryotic host as provided herein. Only based on this knowledge provided by the present application the skilled person is be able to adjust/optimize the assay conditions used to remove or reduce the conformational variant present in a composition comprising the desired ISVD polypeptide product and the product-related conformational variant. The identification of the conformational variant of a polypeptide comprising or consisting of at least three or at least four ISVDs by the specific methods provided herein (see “Analytical methods” in the below section) is thus a prerequisite to allow the skilled person to specifically adjust/optimize prior art purification methods such that the conformational variant can be specifically removed.

The desired polypeptide product is isolated/purified by applying conditions that remove the conformational variant from the composition comprising the desired polypeptide product and the conformational variant thereof. In this aspect, the conformational variant is removed by one or more preparative chromatographic techniques. The chromatographic technique can be a preparative chromatographic technique based on hydrodynamic volume, surface charge and/or hydrophobic exposure/surface hydrophobicity. In one embodiment, the preparative chromatographic technique is selected from size exclusion chromatography (SEC), ion-exchange chromatography (IEX), e.g. cation-exchange chromatography (CEX), mixed-mode chromatography (MMC), and hydrophobic interaction chromatography (HIC).

According to one embodiment, the conformational variant is removed by a preparative chromatographic separation based on hydrodynamic volume. Accordingly, the conformational variant is removed using preparative size-exclusion chromatography (SEC). In SEC, the chromatography column is packed with fine, porous beads which are composed of (without being limiting) dextran polymers (Sephadex), agarose (Sepharose), or polyacrylamide (Sephacryl or BioGel P). The pore sizes of these beads are used to estimate the dimensions of macromolecules. Without being limiting, examples of SEC resins include the Sephadex based products (GE Healthcare, Merck), Bio-gel based products (Bio-Rad), Sepharose based products (GE Healthcare), and Superdex based products (GE Healthcare).

In another embodiment, the conformational variant is removed by a preparative chromatographic separation based on surface charge. Accordingly, the conformational variant is removed using preparative ion-exchange chromatography (IEX) (e.g, cation exchange chromatography (CEX)). Without being limiting, examples of IEX resins include Poros 50HS (ThermoFischer), Poros 50HQ (ThermoFischer), SOURCE 30S (GE Healthcare), SOURCE 15S (GE Healthcare), SP Sepharose (GE Healthcare), Capto S (GE Healthcare), Capto SP Impres (GE Healthcare), Capto S ImpAct (GE Healthcare), Q Sepharose (GE Healthcare), Capto Q (GE Healthcare), DEAE Sepharose (GE Healthcare), Poros XS (Thermo Scientific™), AG® 50W (Bio-Rad), AG® MP-50 (Bio-Rad), Nuvia HR-S (Bio-Rad), UNOsphere™ S (Bio-Rad), and UNOsphere Rapid S (Bio-Rad).

In another embodiment, the conformational variant is removed by a preparative chromatographic separation based on surface hydrophobicity/hydrophobicity exposure. Accordingly, the conformational variant is removed using preparative hydrophobic interaction chromatography (HIC). In one embodiment, the HIC is based on a HIC column resin. Without being limiting, the HIC resin can be selected from Capto Phenyl ImpRes (GE Healthcare), Capto Butyl ImpRes (GE Healthcare), Phenyl HP (GE Healthcare), Capto Butyl(GE Healthcare), Capto Octyl (GE Healthcare), Toyopearl PPG-600 (Tosoh Biosciences), Toyopearl phenyl-600 (Tosoh Biosciences), Toyopearl phenyl-650 (Tosoh Biosciences), Toyopearl butyl-600 (Tosoh Biosciences), Toyopearl butyl-650 (Tosoh Biosciences), TSKgel Phenyl 5-PW (Tosoh Biosciences). In another embodiment the HIC is based on a HIC membrane. Without being limiting, the HIC membrane can be an Adsorber Q (GE Healthcare), Adsorber S (GE Healthcare), Adsorber Phen (GE Healthcare), Mustang Q systems (Pall), NatriFlo HD-Q membrane chromatography (Natrix Separations), Sartobind STIC (Sartorius), Sartobind Q (Sartorius), or Sartobind Phenyl (Sartorius).

In still another embodiment, the conformational variant is removed by a preparative chromatographic separation based on hydrodynamic volume, surface charge, and/or surface hydrophobicity/hydrophobicity exposure. Accordingly, the conformational variant is removed using mixed-mode chromatography (MMC). MMC refers to chromatographic methods that utilize more than one form of interaction between the stationary phase and analytes in order to achieve their separation. MMC resins therefore are based on media that have been functionalized with ligands inherently capable of several different types of interaction: ion exchange, affinity, size exclusion, and hydrophobic.

Various hydroxyapatite chromatographic resins are available commercially, and any available form of the material can be used. A detailed description of the conditions suitable for hydroxyapatite chromatography is provided in WO 2005/044856 and WO 2012/024400, the contents of which are incorporated by reference herein in its entirety.

In one embodiment, the hydroxyapatite is in a crystalline form. The hydroxyapatites may be agglomerated to form particles and sintered at high temperatures into a stable porous ceramic mass. The particle size of the hydroxyapatite may vary widely, but a typical particle size ranges from 1 μm to 1000 μm in diameter, and may be from 10 μm to 100 μm. In one embodiment, the particle size is 20 μm. In another embodiment, the particle size is 40 μm. In yet another embodiment, the particle size is 80 μm.

A number of chromatographic supports may be employed in the preparation of ceramic hydroxyapatite columns, the most extensively used are Type I and Type II hydroxyapatite. Type I has a high protein binding capacity and better capacity for acidic proteins. Type II, however, has a lower protein binding capacity, but has better resolution of nucleic acids and certain proteins. The Type II material also has a very low affinity for albumin and is especially suitable for the purification of many species and classes of immunoglobulins. The choice of a particular hydroxyapatite type can be determined by the skilled person.

Without being limiting, the hydroxyapatite resin is CHT Ceramic Hydroxyapatite, Type I (20, 40 or 80 μm) (BioRad), CHT ceramic hydroxyapatite type II (20, 40 or 80 μm) (BioRad), MPC™ Ceramic Hydroxyfluoroapatite Type I (40 μm), Ca⁺⁺Pure-HA (Tosoh BioScience).

In addition, or in the alternative, the conformational variant can be removed using any sequential combination of the aforementioned preparative SEC, IEX, HIC, or MMC.

In view of the present disclosure the skilled person will be able to find suitable chromatography conditions to identify and then remove (or at least reduce) the conformational variant of a multivalent ISVD polypeptide. Having identified the conformational variant described herein, the skilled person will be able to adapt the parameters and conditions (gradient, buffer, concentrations) of the selected chromatographic method and subsequently take the appropriate fraction of the peak(s). For example, but not being limited thereto, the chromatography conditions used in the examples herein can be used for the removal (or at least reduction) of the conformational variant of a multivalent ISVD polypeptide comprising at least three or at least four ISVDs. The chromatography conditions used in the examples can at least serve as reference point for the development of suitable chromatography conditions to remove (or at least reduce) the conformational variant of a particular multivalent ISVD polypeptide comprising at least three or at least four ISVDs.

Specifically, based on the teaching in the present application, removal or reduction of the conformational variant from a composition comprising both the multivalent ISVD polypeptide and the conformational variant thereof comprises the steps of:
i) applying a preparative chromatographic technique;
ii) analysing the fractions obtained from step (i) for the presence of the multivalent ISVD polypeptide;
iii) selecting those fractions of step (ii) which only comprise the multivalent ISVD polypeptide but not the conformational variant.

Steps i) and ii) can be performed by means known to the skilled person in the field of antibody purification, specifically in the field of ISVD purification. The method can be specifically adapted/optimized for both identification of the conformational variant and removal/reduction of the conformational variant as provided herein. Suitable exemplary analytical and preparative chromatographic techniques are described herein. These general techniques have to be specifically adapted/optimized to allow removal/reduction of the conformational variant.

Steps ii) and iii) can be accomplished by the specific analytical chromatographic techniques described in section 5.4.5 below. For instance, a chromatographic fraction only comprises the multivalent ISVD polypeptide but not the conformational variant if there is no post-peak shoulder and/or separate post-peak, detectable in (analytical) SE-HPLC. The presence of the conformational variant can be also excluded if there is no pre-peak shoulder and/or separate pre-peak, or if there is no post-peak shoulder and/or separate post-peak detectable in analytical IEX-HPLC.

In the prior art the skilled person had no knowledge about the existence of a conformational variant of a polypeptide comprising or consisting of at least three or at least four ISVDs when produced in lower eukaryotic host as provided herein. Only based on this knowledge provided by the present application the skilled person can adjust/optimize steps i) to iii) above such that the specific removal/reduction of the conformational variant can be achieved.

Fractions containing the conformational variant can be discarded or can be treated according to the conversion methods described herein (section 5.4.3 “Conversion of the conformational variant into the desired polypeptide product”) to convert the conformational variant into the desired polypeptide product. The success of the conversion can be evaluated as described herein, e.g. by the analytical chromatographic techniques described in section 5.4.5 below.

The fractions only comprising the multivalent ISVD polypeptide obtained after step iii) can optionally be subject to further purification or filtration steps as known in the art.

A fraction is considered as “only comprising the multivalent ISVD polypeptide (but not the conformational variant)” if there is essentially no post-peak shoulder and/or separate post-peak detectable in (analytical) SE-HPLC. Alternatively, a fraction is considered as “only comprising the multivalent ISVD polypeptide (but not the conformational variant)” if there is essentially no pre-peak shoulder and/or separate pre-peak or if there is essentially no post-peak shoulder and/or separate post-peak detectable in analytical IEX-HPLC. “Essentially no pre-peak shoulder and/or separate pre-peak” or “essentially no post-peak shoulder and/or separate post-peak” means that the ratio of the area under the curve (AUC) for the pre-peak/post-peak (shoulder) to the total area under the curve of the main peak and the pre-peak/post-peak (shoulder) in the respective SE-HPLC or IEX-HPLC chromatogram is lower than 5%, e.g., 4.5% or lower, 4% or lower, 3% or lower, 2% or lower, or even 1% or lower. In one embodiment, there is no pre-peak/post-peak (shoulder) detectable in the respective SE-HPLC or IEX-HPLC chromatogram.

In another aspect, the conformational variant is removed or reduced by applying the composition comprising the multivalent ISVD polypeptide and the conformational variant to a chromatography column using a load factor of at least 20 mg protein/ml resin. In one embodiment of this aspect, the load factor is at least 30 mg protein/ml resin, or at least 45 mg protein/ml resin. In one embodiment, the chromatographic column is a Protein A column. Accordingly, the conformational variant is removed or reduced by applying the composition comprising the multivalent ISVD polypeptide and the conformational variant to a Protein A column using a load factor of at least 20 mg protein/ml resin. In another embodiment, the conformational variant is removed or reduced by applying the composition comprising the multivalent ISVD polypeptide and the conformational variant to a Protein A column using a load factor of at least 45 mg protein/ml resin.

The chromatographic technique(s) used to remove (or reduce) the conformational variant from a composition comprising the ISVD polypeptide and the conformational variant thereof may be applied on culture supernatant comprising the multivalent ISVD polypeptide. For example, the capture step can be used for the removal or reduction. The chromatographic technique used to remove (or reduce) the conformational variant may be also applied on a partially or highly purified preparation of the multivalent ISVD polypeptide. For example, the chromatographic technique used to remove (or reduce) the conformational variant can be applied after the capture step, but before or at the first polish step, or at one or more further polish steps, or after the polish steps.

### 5.4.5 Analytical Methods

### Analytical Methods Used to Observe the Conformational Variant

The conformational variant of a polypeptide comprising or consisting of at least three or at least four ISVDs can be identified by the specific analytical chromatographic techniques provided herein. Analytical chromatographic methods are known to the skilled person, such as analytical SE-HPLC and IEX-HPLC. These methods, however, need to be adapted/optimized to the problem of identifying the conformational variant. A prerequisite for adaption/optimization of such analytical chromatographic techniques is thus the knowledge that the production of a polypeptide comprising or consisting of at least three or at least four ISVDs in lower eukaryotes can result (partially) in a conformational variant as described herein.

As provided herein, the conformational variant can be distinguished from the desired polypeptide product based on a decreased hydrodynamic volume. The presence of the conformational variant can thus be detected by analytical SE-HPLC. Using suitable conditions, the presence of the conformational variant is demonstrated in the SE-HPLC chromatogram by a post-peak shoulder or a separate post peak. Hence, SE-HPLC adapted/optimized for identification of the conformational variant can be used to validate the conditions that convert the conformational variant into the desired polypeptide product, as described herein. Moreover, SE-HPLC adapted/optimized for identification of the conformational variant can be used to validate the removal or reduction of the conformational variant from a composition comprising the desired polypeptide product and the conformational variant thereof.

As further provided herein, the conformational variant can be distinguished from the desired polypeptide product based on an altered surface charge and/or surface hydrophobicity. Using suitable conditions, the presence of the conformational variant can thus be detected by (specifically developed) analytical IEX-HPLC. Depending on the quality of the alteration in surface charge and/or surface hydrophobicity, the presence of the conformational variant can be demonstrated in the IEX-HPLC chromatogram by a pre/post-peak shoulder or a separate pre/post-peak. Hence, IEX-HPLC adapted/optimized for identification of the conformational variant can be used to validate the conditions that convert the conformational variant into the desired polypeptide product. Moreover, IEX-HPLC adapted/optimized for identification of the conformational variant can used to validate the removal or reduction of the conformational variant from a composition comprising the desired polypeptide product and the conformational variant thereof.

Based on the present disclosure, the skilled person will be able to find suitable chromatography conditions to identify the conformational variant of the multivalent ISVD polypeptide. For example, but not being limited thereto, the chromatography conditions used in the examples herein can be used for detection of the conformational variant of a multivalent ISVD polypeptide comprising at least three or at least four ISVDs. The chromatography conditions used in the examples herein can at least serve as reference point for the development of suitable chromatography conditions to detect the conformational variant for a particular multivalent ISVD polypeptide comprising at least three or at least four ISVDs. Basic exemplary conditions are provided in Table C.

### Further Analytical Methods Used for Characterization of the Conformational Variant

The following analytical techniques are known to the skilled person. For example, but without being limited thereto, suitable conditions are provided in Table C.

TABLE C
Exemplary analytical methods for the detection and
characterization of multivalent ISVD polypeptides.

| Method | Materials | Buffer | Method conditions |
|---|---|---|---|
| SE-HPLC | Xbridge | Mobile phase: 750 mM L- | Column temperature: |
| | Protein BEH | arginine + 10 mM | 25° C. |
| | SEC200A, 7.8 × | phosphate + 0.02% NaN3 | UV detection |
| | 300 mm, 3.5 | pH 7.0 | Flow rate: 0.6 or 1 mL/min |
| | μm, 200 Å | | Elution mode: isocratic |
| IEX-HPLC | ProPac Elite | Mobile phase A: 20 mM | Column temperature: |
| (protocol | WCX, 4 × | Mops pH 7.9 + 10% | 25° C. |
| I) | 250 mm | MeOH | UV detection |
| | | Mobile phase B: 20 mM | Flow rate: 0.6 mL/min |
| | | Mops pH 7.9 + 10% | Elution mode: gradient |
| | | MeOH + 0.5M NaCl | |

| Time (min) | % buffer B |
|---|---|
| 0.00 | 9 |
| 5.40 | 9 |
| 25.40 | 54 |
| 27.90 | 100 |
| 30.10 | 100 |
| 30.80 | 9 |
| 32.90 | 9 |

| | | | |
|---|---|---|---|
| IEX-HPLC | Achrom YMC- | Buffer A: 20 mM MOPS | Column temperature: |
| (protocol | BioPro SP-F, | pH 7.0 20 mM NaCl | 30° C. |
| II) | 3 μm, 100 × 4.6 | Buffer B: 20 mM MOPS; | UV detection |
| | mm | 0.25M NaCl pH 7.0 | Flow rate: 0.5 mL/min |
| | | | Elution mode: gradient |

| Time (min) | % buffer B |
|---|---|
| 0.0 | 10 |
| 3.4 | 10 |
| 18.15 | 43 |
| 19.8 | 100 |
| 21.5 | 100 |
| 22.0 | 10 |
| 25 | 10 |

| | | | |
|---|---|---|---|
| CGE | Bare Fused | Reduced CGE Master | 30 min separation (15 KV) |
| | Silica Capillary, | mix: SDS-MW Sample | |
| | internal | Buffer 75 μL, 2- | |
| | diameter 50 | mercaptoethanol 5 μL | |
| | μm | | |

### Analytical Methods Used to Observe Potency of the ISVD Polypeptide

The conformational variant may also be distinguished from the desired polypeptide product by an alteration in potency, wherein the conformational variant has a decreased potency compared to the desired polypeptide product. Moreover, the (successful) conversion of the conformational variant into the desired polypeptide product can be demonstrated by partial or full recovery of the potency relative to the potency of the respective desired polypeptide product or relative to a reference ISVD polypeptide which was not enriched or depleted for the conformational variant.

Potency in this regard refers to the binding capacity (towards a particular target) of, the functional activity of, and/or the amount of polypeptide required to produce a particular effect by one or more of the at least three or at least four ISVDs present in the polypeptide. Potency can be measured in an in vitro assay (e.g. competitive ligand binding assay or cell-based assay) or in vivo (e.g. in an animal model). Without being limited thereto, potency may refer to the inhibition of TNFα-induced expression of the luciferase reporter gene, inhibition of the IL-23 induced expression of the luciferase reporter gene, inhibition of OX40 L induced expression of the luciferase reporter gene, or binding capacity to human serum albumin. Suitable, exemplary assays to determine the potency differences between the desired polypeptide product and the conformational variant thereof are (without being limiting):

Cell based reporter assay for the potency testing of the TNF-alpha binding moiety Glo Response™ HEK293_NFkB-NLucP cells are TNF receptor expressing cells that were stably transfected with a reporter construct encoding Nano luciferase under control of a NFκB dependent promoter. Incubation of these cells with soluble human TNFα results in NFκB mediated Nano-luciferase gene expression.

The assay may be generally performed as follows. The Glo Response™ HEK293_NFkB-NLucP cells are to be seeded at suitable cell number in normal growth medium in suitable tissue culture plates. Dilution series of the ISVD construct to be tested are added to a suitable and sufficient amount of human TNFα and incubated with the cells for a sufficient time (e.g. about 5 hours) at 37° C. and 5% CO₂. During this incubation, TNF-induced expression of the luciferase reporter gene is inhibited by the ISVD construct. After the incubation, the plates are cooled down (e.g., for 10 minutes) before addition of the Nano-Glo Luciferase substrate to quantify luciferase activity. Five minutes after addition of the substrate, luminescence can be measured on e.g., a Tecan Infinite F-plex plate reader. Luminescence, expressed as relative light units (RLU), is directly proportional to the concentration of luciferase.

### Cell Based Reporter Assay for the Potency Testing of the IL-23 Binding Moiety

Glo Response™ HEK293_human IL-23R/IL-12Rb1-Luc2P are cells which have been stably transfected with a reporter construct containing the luciferase gene under control of the sis-inducible element (SIE) responsive promotor. Additionally, these cells constitutively overexpress both subunits of the human IL-23 receptor, i.e. IL-12Rb1 and IL-23R. Stimulation of these cells with human IL-23 induces expression of the luciferase reporter gene.

The assay can be generally performed as follows. The Glo Response™ HEK293_human IL-23R/IL-12Rb1-Luc2P cells are to be seeded at suitable cell number in normal growth medium in suitable tissue culture plates. Serial dilutions of the ISVD construct to be tested is added to the cells, followed by the addition of suitable amount of recombinant hIL-23 (e.g., 3 pM). Cells are to be incubated for a sufficient time (e.g., about 6 hours) at 37° C. After the incubation step, a cooling down period of the plates (e.g., 10 minutes) is required before addition of the luciferase substrate 5′-fluoroluciferin (Bio-Glo™ Luciferase Assay System) to quantify the luciferase activity. Five minutes after addition of the substrate, luminescence can be measured e.g., on a Tecan Infinite F-plex plate reader. Luminescence (expressed as relative light units, RLU) is directly proportional to the concentration of luciferase.

### Cell Based Reporter Assay for the Potency Testing of the OX40 L Binding Moiety

Potency towards inhibition of OX40 L can be assessed using a cell-based reporter assay. For example, Glo Response™ NFkB-luc2/OX40 Jurkat suspension cells are to be seeded at suitable cell number in normal growth medium in suitable tissue culture plates. Dilution series of the ISVD construct are added to the cells followed by a fixed concentration of 700 pM OX40 L. The plates are then to be incubated for a sufficient time (e.g., 3 hours) at 37° C. and 5% CO₂ in an incubator to allow activation of the NF-kB promotor by OX40 L/OX40 signaling, which in turn results in transcription of the luciferase gene. After the incubation step, a cooling down period of the plates (e.g., 10 minutes) is required before addition of the luciferase substrate 5′-fluoroluciferin (Bio-Glo™ Luciferase Assay System) to quantify the luciferase activity. Five minutes after addition of the substrate, luminescence can be measured e.g., on a Tecan Infinite F200 plate reader. Luminescence (expressed as relative light units, RLU) is directly proportional to the concentration of luciferase.

### ELISA Based Albumin Binding Assay for Potency Testing of the Albumin Binding Moiety

Binding potency to human serum albumin (HSA) can be measured by direct binding ELISA. For example, 96-well microtiter plates can be coated overnight with a suitable amount of HSA in bicarbonate buffer at pH 9.6. Non-specific binding sites on the plates can be blocked for about 30 minutes at room temperature (RT) using Superblock T20. Serial dilutions of the ISVD construct are prepared in PBS+10% Superblock T20 and transferred to the HSA coated plates, followed by an incubation step of about 75 min at RT while shaking at 600 rpm. Bound ISVD construct can be detected using e.g., 1 μg/mL of a mouse anti-ISVD construct antibody for 90 min at RT while shaking at 600 rpm, followed by a 50 min incubation with 0.2 μg/mL horse radish peroxidase (HRP)-labelled polyclonal rabbit anti-mouse antibody at RT while shaking at 600 rpm. Bound HRP-labelled polyclonal antibody can be measured by addition of ⅓ diluted 3,5,3′5′-tetramethylbenzidine (TMB) one. The resulting chromogenic reaction between HRP and the substrate is stopped by addition of 1M HCl. The optical density can be measured at a wavelength of 450 nm and a reference wavelength of 620 nm, using e.g., a plate-spectrophotometer. This OD is directly proportional to the amount of ISVD construct bound to the coated HSA.

### 5.5 Multivalent ISVD Polypeptide Products Obtainable by the Production and/or Isolation or Purification Method

The present application also describes improved compositions comprising the multivalent ISVD polypeptide product obtainable by the methods as described herein. It is characterized by a reduced level, or the complete absence, of the product-related conformational variant. For example, the ISVD polypeptide obtainable by the methods described herein comprises less than 5%, e.g. 0-4.9%, 0-4%, 0-3%, 0-2% or 0-1% product-related conformational variant. In another embodiment, the ISVD polypeptide obtainable by the methods described herein comprises less than 1%, less than 0.5%, less than 0.01% of the product-related conformational variant. In one embodiment, the multivalent ISVD polypeptide product obtainable by the method described herein is free of the product-related conformational variant. For example, the composition comprising the ISVD polypeptide obtainable by the methods described herein comprises less than 5%, e.g. 0-4.9%, 0-4%, 0-3%, 0-2% or 0-1% product-related conformational variant. In another embodiment, the composition comprising the ISVD polypeptide obtainable by the methods described herein comprises less than 1%, less than 0.5%, less than 0.01% of the product-related conformational variant. In one embodiment, the composition comprising the multivalent ISVD polypeptide product obtainable by the method described herein is free of the product-related conformational variant. The skilled person can readily determine the proportion of product-related conformational variant as a % of the total polypeptide (i.e. by determining AUC of pre-peak or post-peak (shoulder)/total AUC of both main peak and pre-peak or post-peak (shoulder)) e.g., by SE-HPLC or IEX-HPLC as described herein.

In other words, the multivalent ISVD polypeptide product obtainable by the methods described herein is characterized by an improved structural homogeneity as compared to prior art preparations. In particular, prior art preparations may comprise 5% or higher proportions of product-related conformational variant, such as 5-15%, 5-20%, 5-25% or even higher proportions of product-related conformational variant.

In view of the improved structural homogeneity, the multivalent ISVD polypeptide product obtainable by the methods is advantageous as compared to prior art preparations. For example, the multivalent ISVD polypeptide product obtainable by the present methods is advantageous for therapeutic applications. In the connection of therapeutic antibody use, structural homogeneity is of foremost clinical and regulatory importance.

Accordingly, the present application also describes pharmaceutical preparations and other compositions comprising the multivalent ISVD polypeptide product obtainable by the methods described herein. The multivalent ISVD polypeptide product obtainable by the method described herein can also be used in therapy (i.e. medical use).

The skilled person can readily formulate pharmaceutically suitable formulations of the multivalent ISVD polypeptide product obtainable by the method described herein on the basis of common general knowledge. Moreover, the references specifically dealing with multivalent ISVD polypeptides, which are cited herein, are explicitly referred to. Without limitation, formulations for standard routes of application can be prepared, including formulations for nasal, oral, intravenous, subcutaneous, intramuscular, intraperitoneal, intravaginal, rectal application, topical application or application by inhalation.

The skilled person can also readily devise suitable methods of treatment characterized by the use of a therapeutically effective amount of the multivalent ISVD polypeptide obtainable by the methods described herein.

### 6 EXAMPLES

The following Examples describe the identification of the presence of a conformational variant of multivalent ISVD constructs during the production and purification process. It was shown that such conformational variants exhibit a distinguishing biochemical/biophysical behaviour allowing for their separation via chromatographic methods. Also, it could be revealed that, apart from the differences in biochemical/biophysical properties, the conformational variant showed differences in the potency of one or more ISVD building blocks towards their respective target. Eventually, it could be shown that such undesired conformational variant can be converted into the intact ISVD polypeptide by suitable treatment conditions and/or can be specifically decreased/removed from a composition containing both the intact form as well as the undesired conformational variant during the purification process of the ISVD constructs.

### 6.1 Example 1: Identification of a Conformational Variant of Compound A

A Conformational Variant could be Identified During the Capture Process Step of a Multivalent ISVD Construct A conformational variant of multivalent ISVD constructs was identified during the first step of purification (i.e., the capture process step) of multivalent ISVD constructs. The capture process step was performed to recover a maximum of the ISVD product from the clarified supernatant.

During the capture purification process of compound A (SEQ ID NO: 1), it was observed that the analytical size exclusion profile (SE-HPLC; conditions as set forth in Table C) was different depending on the resin and the elution buffer used during the chromatographic process.

Compound A (SEQ ID NO: 1) is a multivalent ISVD construct comprising three different sequence optimized variable domains of heavy-chain llama antibodies that bind to three different targets. The ISVD building blocks are fused head-to-tail (N-terminus to C-terminus) with a G/S linker in the following format: an OX40 L-binding ISVD—9GS linker—an OX40 L-binding ISVD—9GS linker—a TNFα-binding ISVD—9GS linker—a human serum albumin-binding ISVD—9GS linker—a TNFα-binding ISVD and have the following sequence:

TABLE 1
Amino acid sequence of compound A.
Compound A (SEQ ID NO: 1)

DVQLVESGGGVVQPGGSLRLSCAASGRTFSSIYAKGWFRQAPGKEREFVAAISRSGRSTSYADSVKGRFT
ISRDNSKNTVYLQMNSLRPEDTALYYCAAVGGATTVTASEWDYWGQGTLVTVSSGGGGSGGGSEVQL
VESGGGVVQPGGSLRLSCAASGRTFSSIYAKGWFRQAPGKEREFVAAISRSGRSTSYADSVKGRFTISRD
NSKNTVYLQMNSLRPEDTALYYCAAVGGATTVTASEWDYWGQGTLVTVSSGGGGSGGGSEVQLVESG
GGVVQPGGSLRLSCAASGFTFSDYWMYWVRQAPGKGLEWVSEINTNGLITKYPDSVKGRFTISRDNAK
NTLYLQMNSLRPEDTALYYCARSPSGFNRGQGTLVTVSSGGGGSGGGSEVQLVESGGGVVQPGGSLRL
SCAASGFTFRSFGMSWVRQAPGKGPEWVSSISGSGSDTLYADSVKGRFTISRDNSKNTLYLQMNSLRPE
DTALYYCTIGGSLSRSSQGTLVTVSSGGGGSGGGSEVQLVESGGGVVQPGGSLRLSCAASGFTFSDYWM
YWVRQAPGKGLEWVSEINTNGLITKYPDSVKGRFTISRDNAKNTLYLQMNSLRPEDTALYYCARSPSGFN
RGQGTLVKVSSA

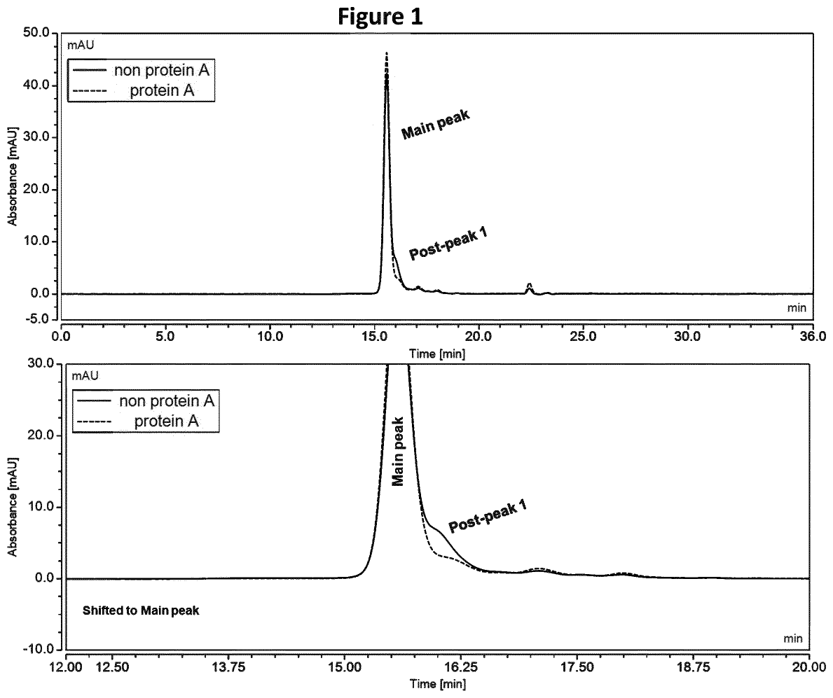

FIG. 1 presents the SE-HPLC profiles for the eluates after chromatography purification on Protein A or non-Protein A capture resins. The SE-HPLC profiles of the eluates showed a less pronounced post-peak shoulder (indicated as post peak 1 in FIG. 1) when using Protein A as capture resin compared to non-Protein A. It was concluded that the presence of the post peak shoulder (post peak 1) is dependent on the conditions/resin used during the chromatographic purification. In contrast to non-Protein A resins, elution on protein A resins is at low pH. Based on these observations, the influence of the elution buffer pH on the SE-HPLC profiles was tested. Accordingly, buffers A to D (described in Table 2) with different acidic pHs were compared for the elution of compound A from Protein A capture resin.

TABLE 2
Elution buffers used for the capture process.

| | pH of elution | | pH post |
|---|---|---|---|
| Buffer | buffer | pH of resulting eluate* | neutralization |
| A | 0.1M Glycine | 2.9 | 6.8 |
| | pH 2.5 | | |
| B | 0.1M Glycine | 3.6 | 6.8 |
| | pH 2.8 | | |
| C | 0.1M Glycine | 4.1 | 6.8 |
| | pH 3.0 | | |
| D | 0.1M Glycine | 4.7 | 6.7 |
| | pH 3.3 | | |

*pH of resulting eluate is slightly higher than the pH of the elution buffer used

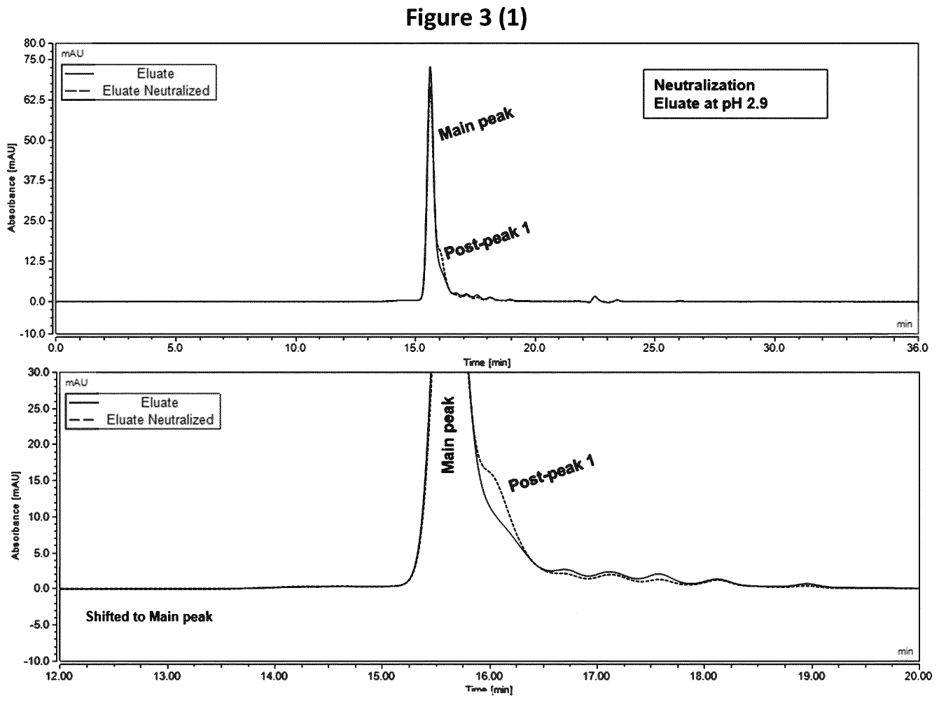

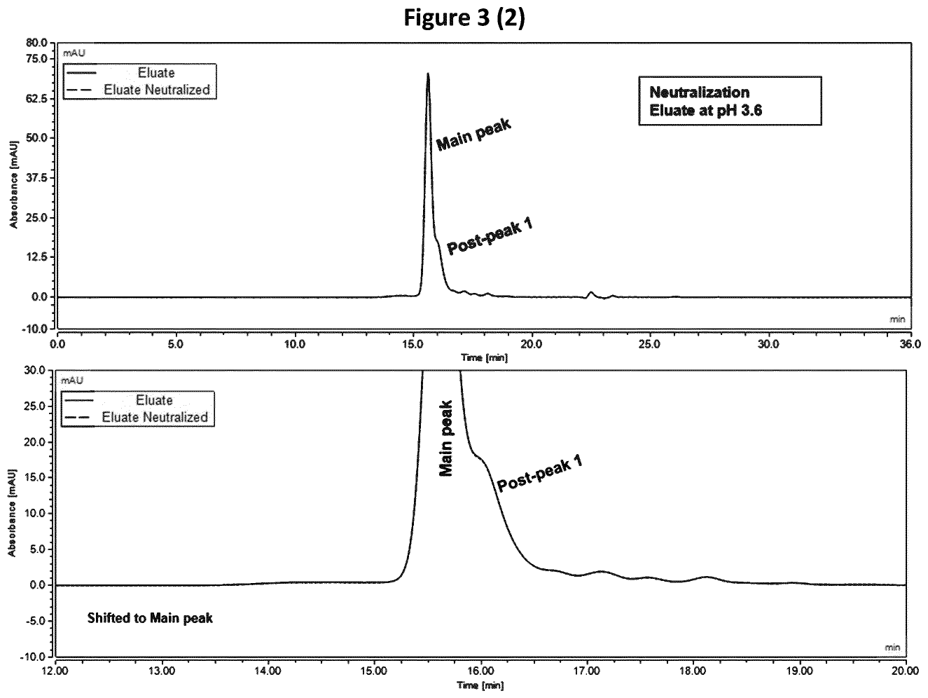

FIG. 2 represents the SE-HPLC profiles for the eluates after Protein A capture and elution using the different elution buffers A, B, C, and D (no neutralization). The post peak 1 was less pronounced in elution buffer A compared to B, C and D. In FIG. 3, the SE-HPLC profiles for capture eluate and capture eluate neutralized to at least a pH of 6.7 using 1M HEPES pH 7.0 directly following elution with elution buffer A (FIG. 3(1)) and elution buffer B (FIG. 3(2)) are presented. The post-peak shoulder (indicated as post peak 1) in the SE-HPLC profile was lower for the eluate at pH 2.9 (buffer A) compared to the eluate at pH 3.6 to 4.7 (Buffer B to D) as seen in FIG. 2 and FIG. 3(1) and FIG. 3(2). However, the post peak 1 was not decreased if the eluate was directly neutralized (compare eluate and neutralized eluate in FIG. 3(1)). Therefore, a “pH hold” could have an effect on the post peak 1. For elution buffer B, the post peak 1 was observed after elution independently of a subsequent neutralization of the resulting eluate (FIG. 3(2)).

Based on these observations, it was concluded that there was an impact of the pH on the detectability of post peak 1 in SE-HPLC. Moreover, it was assumed that post peak 1 may represent a conformational variant of the ISVD construct. The slightly increased retention time may be indicative for a more compact conformation compared to the intact form of the ISVD construct represented by the main peak.

A Conformational Variant could be Identified During the Polish Process Step of a Multivalent ISVD Construct

A conformational variant of the multivalent ISVD construct could also be identified during the polish process step. The polish process step was performed after the capture step to improve the purity of the multivalent ISVD containing composition.

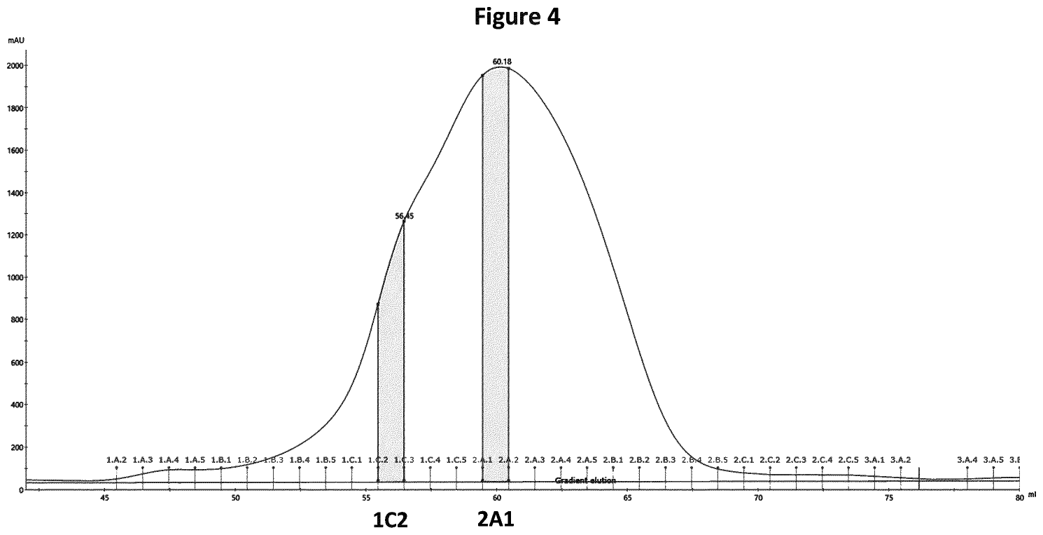

For the polish step of the ISVD construct, a cation exchange chromatography (CEX) was performed. Therefore, a linear salt gradient from 0 to 350 mM NaCl in 25 mM Citrate pH 6.0 was applied over 20 column volumes (CV) at RT on a polish CEX resin. The chromatographic profile is depicted in FIG. 4.

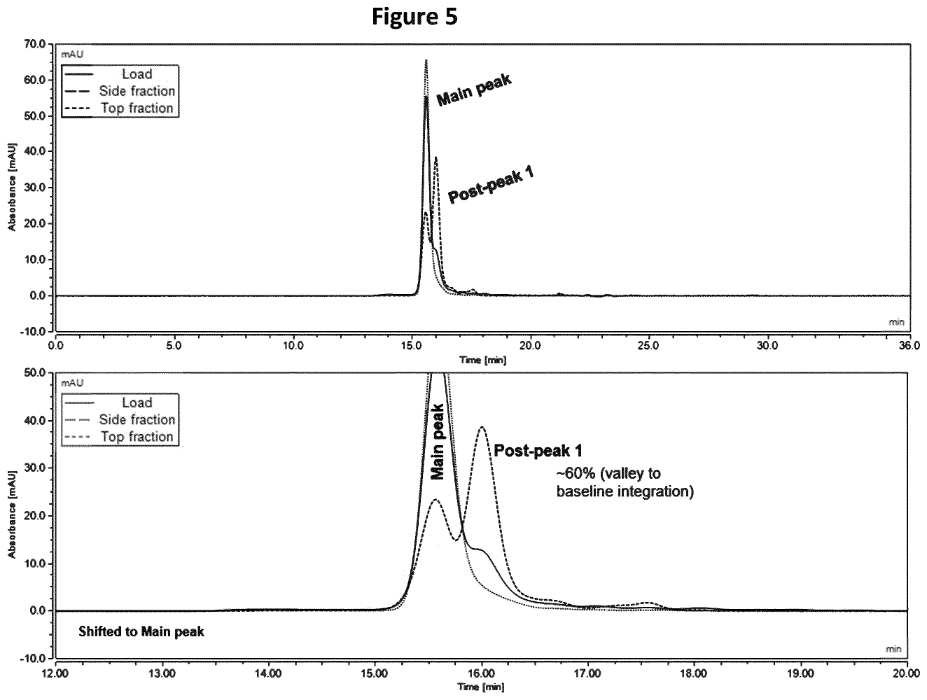

A top fraction (referred to as fraction 2A1 in FIG. 4) as well as a side (front) fraction (referred as fraction 1C2 in FIG. 4) eluting during the linear gradient were further analysed in SE-HPLC and compared to the load material (FIG. 5). The post peak 1 observed in SE-HPLC for the load material was not present for the top fraction of the gradient on CEX resin. In contrast, a significant post peak 1 (approximately 60%) on SE-HPLC was observed for the side (front) fraction.

Hence, a conformational variant of the ISVD construct could be also identified during the polish process step. Distinct eluate fractions of the CEX polish step were shown to contain different proportions of the intact form (main peak) and the conformational variant (post peak 1) in SE-HPLC (FIG. 5). Whereas the top fraction of the CEX polish step was found to be depleted for the conformational variant, the side fraction was rather enriched for it.

The results were similar for different cation exchange resins, such as Capto SP Impres (GE Healthcare) and Capto S ImpAct (GE Healthcare) tested for the polish procedure with a gradient from 0 to 350 mM NaCl in 25 mM citrate pH 6.0 over 20 CV, and for other CEX resins tested for the polish procedure e.g. with a gradient from 0 to 400 mM in 25 mM citrate pH 6.0 over 20 CV (data not shown) and using 25 mM histidine pH 6.0 and a gradient from 0 to 400 mM over 20 CV (data not shown).

These observations further emphasized the conclusions that the post peak 1 observed on SE-HPLC may represent a conformational variant of the ISVD construct. Whereas the slightly increased retention time in SE-HPLC are indicative for a more compact form (i.e., a decreased hydrodynamic volume), the slight difference in retention time observed in preparative CEX are indicative for an altered surface charge compared to the intact ISVD product. There is thus potential to separate the conformational variant and the intact ISVD product using suitable chromatographic techniques, such as preparative SEC or CEX.

### 6.2 Example 2: Verification and Characterization of Conformational Variants of Compound A

In Example 1 it was shown that the compound A elutes as a main peak and a post peak 1 (post-peak shoulder) during analytical SE-HPLC. Due to a slightly longer retention time, it was concluded that the post peak 1 may refer to a more compact form the multivalent ISVD construct. In addition, Protein A affinity chromatography using an elution buffer of pH 2.5 can result in a decreased post peak 1/main peak ratio. However, the post peak 1/main peak ratio remained unchanged if the capture eluate was directly neutralized. It was thus concluded that the conformational variant is convertible into the intact ISVD product and thus does not differ in molecular size.

In order to further characterize the nature of the conformational variant and to exclude the presence of a mass variant, the conformational variant-depleted top fraction and the conformational variant-enriched fraction of the CEX polish of Example 1 was subjected to analysis by analytical ion exchange—high-performance liquid chromatography (IEX-HPLC; conditions as set forth in Table C, protocol I), capillary-electrophoresis isoelectric focusing (CE-IEF) and reverse-phase ultra-high-performance liquid chromatography (RP-UHPLC).

### Behaviour in Analytical IEX-HPLC

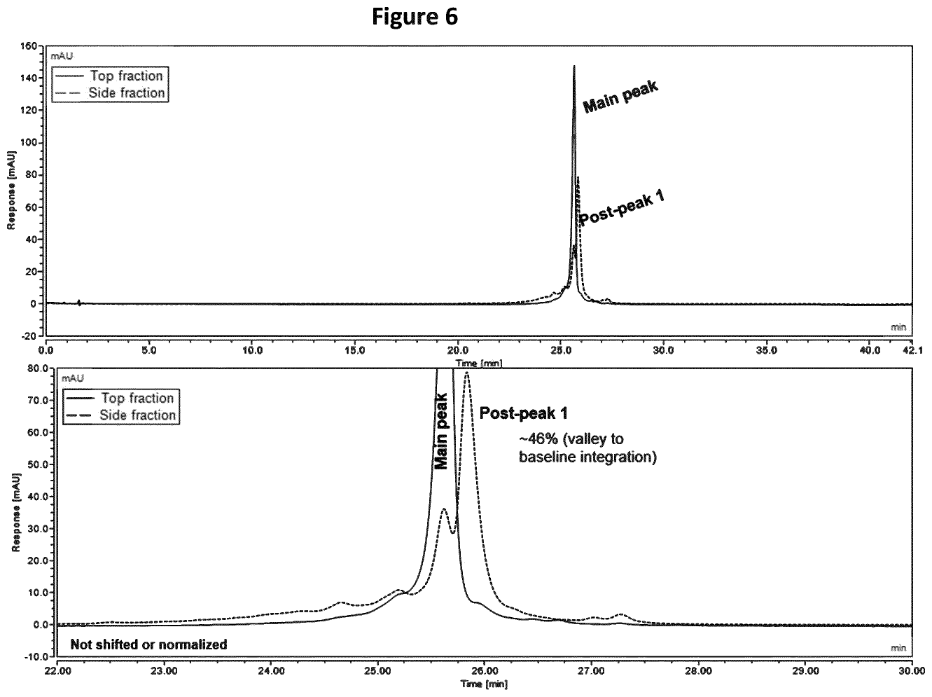

Similar to analytical SE-HPLC, the IEX-HPLC chromatogram showed a significant post peak 1 (approximately 46%) for the conformational variant—enriched side fraction which was not present for the conformational variant-depleted top fraction (FIG. 6).

### Behaviour in CE-IEF/RP-UHPLC

In CE-IEF analytical testing, almost no difference was observed between the side (“enriched”) and top (“depleted”) fractions obtained in the preparative CEX (data not shown). Similarly, no difference for both fractions was observed in RP-UHPLC (data not shown).

In contrast to CE-IEF, IEX-HPLC exhibited a different chromatographic profile between the conformational variant-enriched side CEX fraction and the conformational variant-depleted top CEX fraction. The main difference between the two charge-based methods CE-IEF and IEX-HPLC is that CE-IEF is run in the presence of denaturing conditions (3M urea). No difference in CE-IEF indicates that there are no chemical modifications between the intact ISVD product and the conformational variant leading to overall charge differences. The difference in IEX-HPLC, however, hints for a slightly altered surface charge of the conformational variant compared to the intact ISVD product. In other words, only the surface charge has been altered due to conformational changes whereas the total charge of the molecule was unchanged. These observations also hint at the hypothesis that the conformational variant can be removed by denaturing conditions.

Due to the similar behaviour of both CEX fractions in RP-UHPLC, it was excluded that the compact conformational variant was due to scrambled disulfide bridges compared to the intact form of the ISVD construct.

### Potency Differences of the Intact ISVD Product and its Conformational Variant

To further investigate whether the conformational variant to any extent differs in its potency in target binding, the following assay was performed on the conformational variant-enriched side fraction and on the conformational variant-depleted top fraction obtained from the preparative CEX as described above.

The potencies of the ISVDs towards their respective targets were determined using the following assays (as described in item 5.4.5 above):
Cell based reporter assay for the potency testing of the TNF-alpha binding moiety;
Cell based reporter assay for the potency testing of the OX40 L binding moiety;
ELISA based albumin binding assay for potency testing of the albumin binding moiety.

The results for the potency of the side (“enriched”) and top (“depleted”) fractions are presented in Table 3.

TABLE 3
Potency results for side fraction enriched in conformational variant
and top fraction depleted of conformational variant from polish
CEX gradient. Potency is expressed relatively to a reference which
was not enriched or depleted for the conformational variant.

| Sample | HSA | OX40L | TNF |
|---|---|---|---|
| Side fraction (enriched) | 0.972* | 0.815* | 0.330** |
| Top fraction (depleted) | 0.912* | 0.517* | 1.125* |

*Potency values between 0.5 and 1.5 indicate comparable potency to the reference.
**significant; indicates lower potency than reference.

A significant drop in potency was observed for the conformational variant-enriched fraction compared to the depleted fraction in the TNFα potency assay. Accordingly, the conformational change of compound A impacts the binding potency to TNFα.

### 6.3 Example 3: Determining Conditions that Influence the Conformation of the Compound A

Based on the observations from Example 1 and Example 2 additional experiments were set up to assess the impact of specific experimental conditions that may influence the conformation of the multivalent ISVD construct. The tested conditions were gentle denaturation, stress or the presence of a chaotropic agent. The conditions tested are summarized in Table 4.

TABLE 4
Analytical characterization experiment set up.

| Parameter to be | |
|---|---|
| assessed | Experimental set up |
| Chaotropic agent | Guanidium Hydrochloride (GuHCl) |
| | 0, 1, 2, 3M - 0.5 h incubation |
| Heat stress | 1 and 4 h incubation at 50° C. and 60° C. |
| | Cooling down to room temperature (RT) |
| pH | pH 2.5, 3.0 and 3.5 |
| | with/without pH neutralization after 4 h |
| | incubation at RT |

### Low pH Treatment

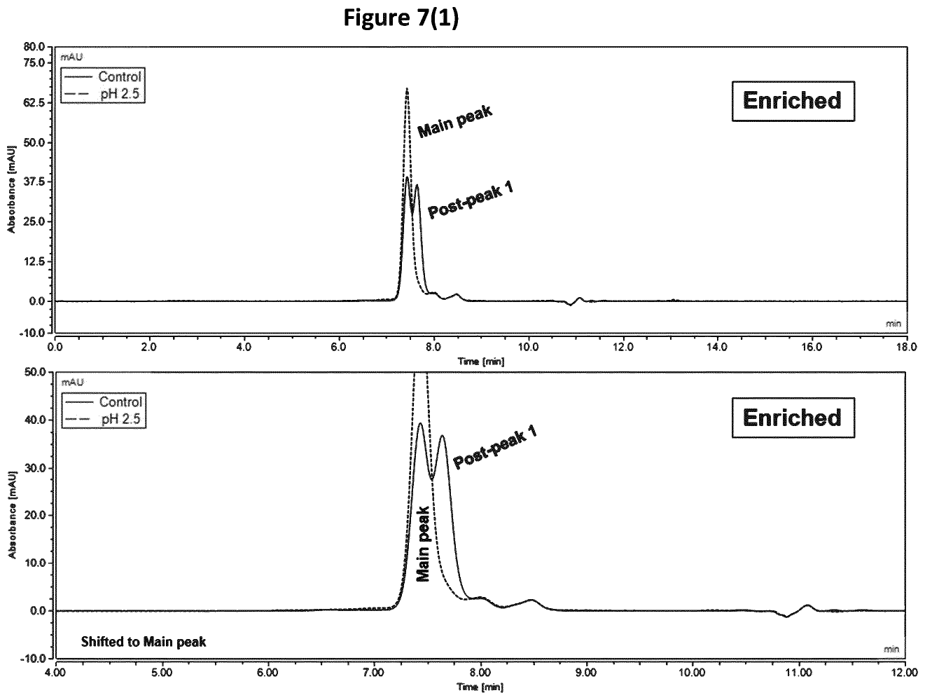

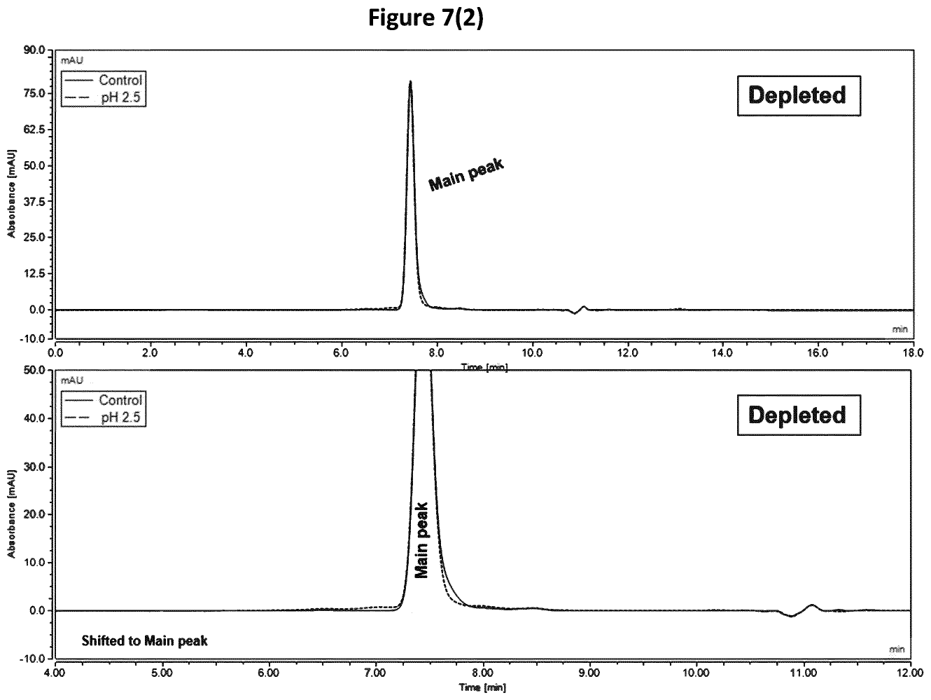

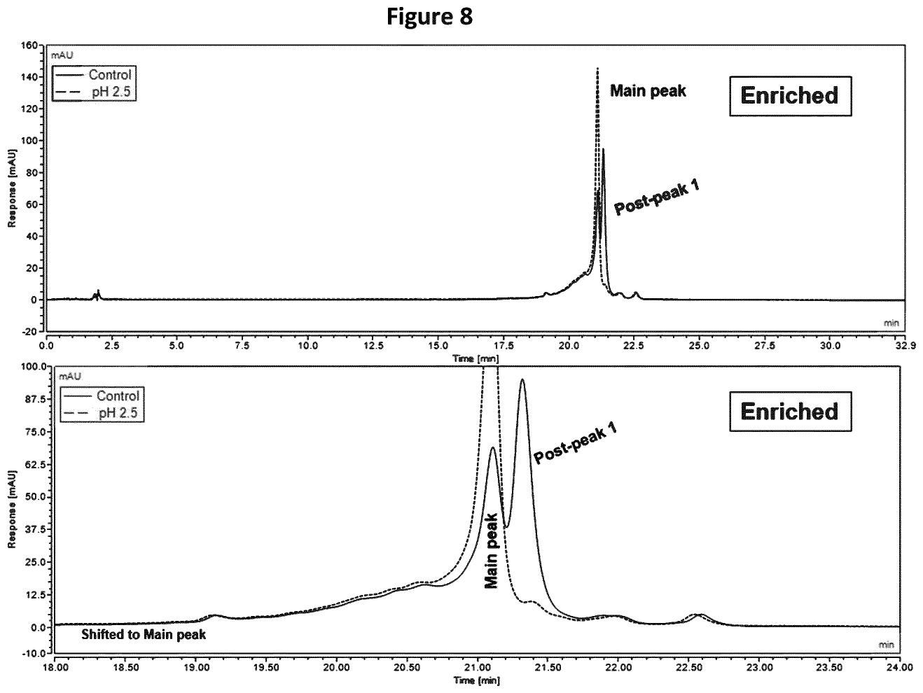

For the low pH treatment, the compact variant enriched and depleted material from the preparative CEX (described above) were treated to reach a final concentration of 100 mM glycine with pH of 2.5, pH of 3.0 or pH of 3.5 or with formulation buffer pH 6.5 (control). Samples were incubated 4 hours at the respective pH and then either directly analysed, or neutralized with 0.1 M NaOH, and then analysed. The impact of treatment at pH 2.5 on the compact variant-enriched and -depleted material was analysed by SE-HPLC and IEX-HPLC (conditions as set forth in Table C; IEX-HPLC protocol I) and are presented in FIGS. 7(1) and (2) (SE-HPLC) and FIG. 8 (IEX-HPLC; compact variant-enriched fraction only).

For the conformational variant enriched material incubated at pH 2.5, the SE-HPLC and IEX-HPLC post peak 1 significantly decreased. As this decrease was associated with an increase of the main peak in both analyses, this demonstrated that the conformational variant was converted to the intact form. Moreover, the conversion was maintained after neutralization when the eluate was incubated at pH 2.5 for 4 hours (data not shown). No change was observed for the control sample or for the conformational variant depleted material (FIG. 7(2); data not shown for IEX-HPLC). For material incubated at pH 3.0 and 3.5, only a small decrease of the SE-HPLC and IEX-HPLC post peaks was observed suggesting that the pH was not low enough to allow the conversion of the conformational variant into the intact form (data not shown).

The stability of the compact variant converted to intact form was then verified after low pH treatment at pH 2.5 and subsequent neutralization. After storage of this compact variant converted into intact form up to 2 weeks at 25° C., there was no change in the SE-HPLC profile demonstrating that the conversion to the intact form upon pH treatment of compact variant-enriched material was maintained (similar to the compact variant-depleted material). The same result was obtained for a 2 weeks storage at 5° C. (data not shown).

Treatment with Chaotropic Agents

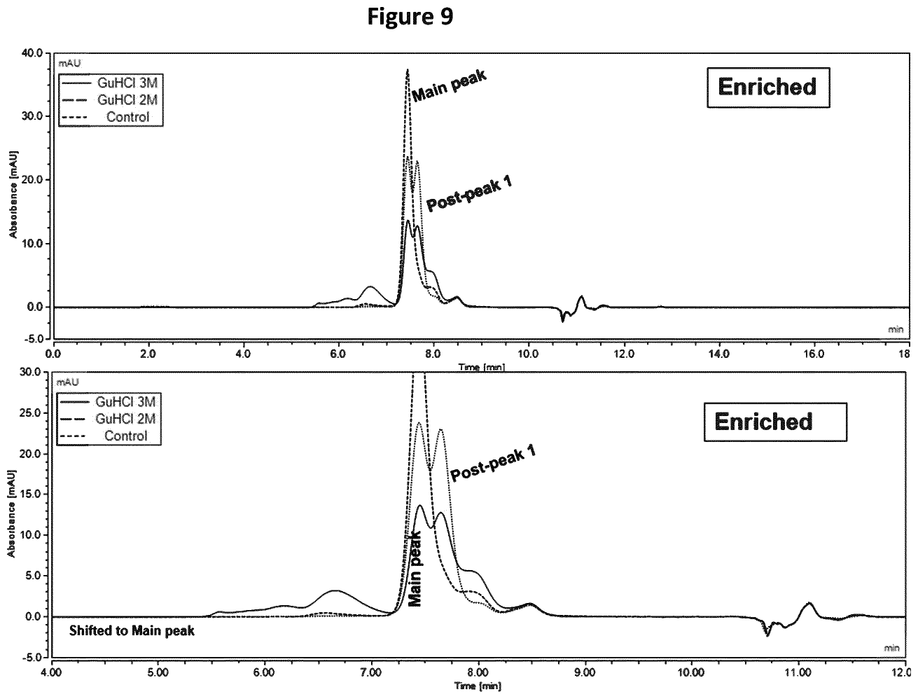

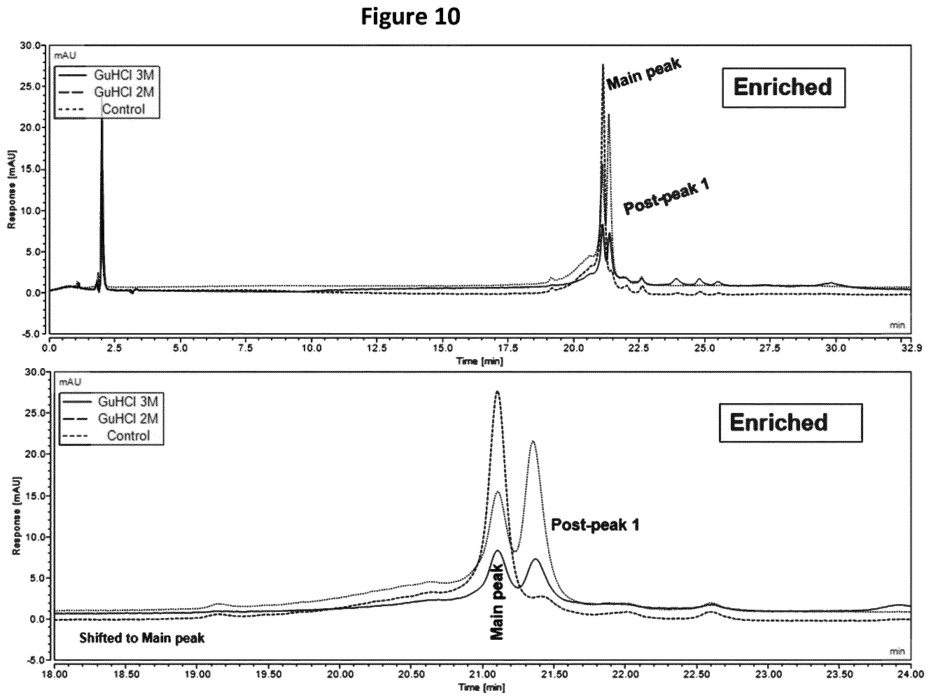

To assess the impact of chaotropic agents, the conformational variant-enriched and -depleted material were incubated for 0.5 hours without or with 1M, 2M, or 3M of Guanidinium chloride (GuHCl) and analysed by SE-HPLC (conditions a set forth in Table C; SE-HPLC) and IEX-HPLC (conditions as set forth in Table C; IEX-HPLC protocol II). The results of the impact of treatment with 2M and 3M GuHCl denaturing agent on the conformational variant enriched material are presented in FIG. 9 (SE-HPLC) and FIG. 10 (IEX-HPLC).

For the compact variant enriched material incubated with GuHCl, the SE-HPLC and IEX-HPLC post peak 1 significantly decreased when a GuHCl concentration of 2M was applied. In addition, the post peak 1 decrease was associated with an increase of the main peak for both analyses, demonstrating that the conformational variant was converted to the intact form. No change was observed for the conformational variant depleted control sample (data not shown).

A concentration of 3M GuHCl was too high for the tested compound A and led to the degradation of the product as demonstrated by the formation of high molecular weight (HMW) species (pre-peak in SE-HPLC).

There was only a slight reduction of the post peaks area in both IEX-HPLC and SE-HPLC analyses upon application of 1M GuHCl. For compound A this condition seemed to be not enough denaturing to fully convert the conformational variant to the intact form (data not shown).

### Heat Stress Treatment

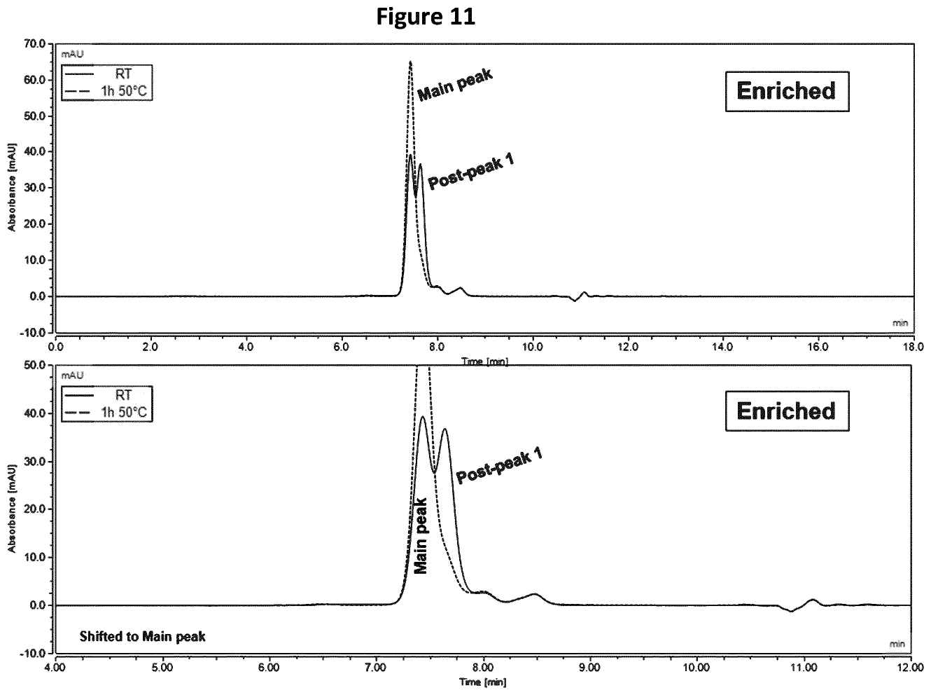

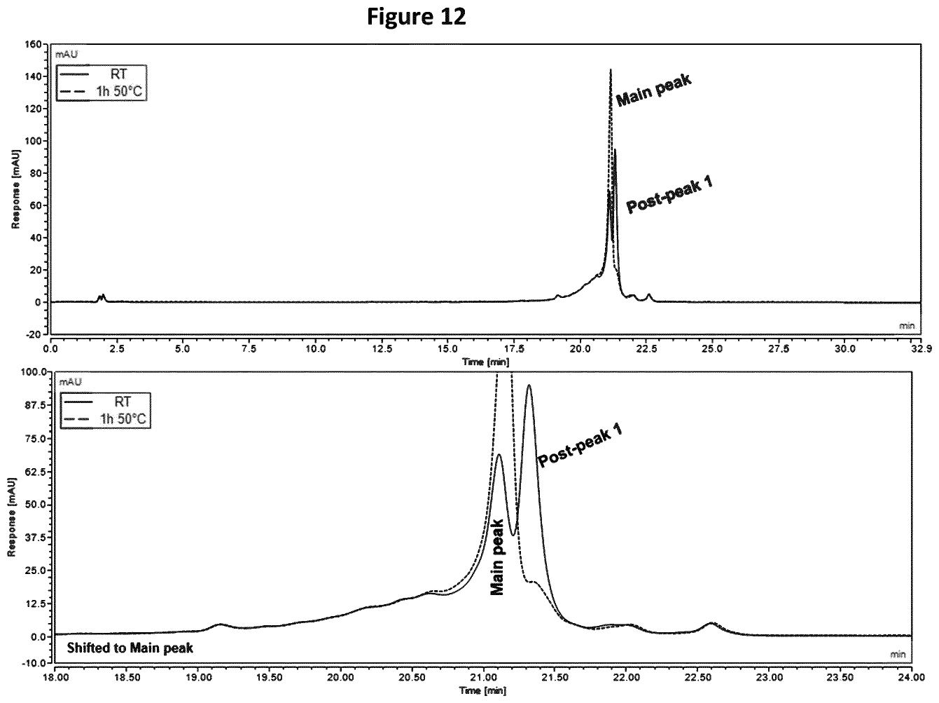

For heat treatment, conformational variant-enriched and -depleted material was incubated for 1 or 4 hours at 50° C. or 60° C., before re-equilibrated to room temperature (RT). The results of the impact of heat stress at 50° C. for 1 hour are presented in FIG. 11 (SE-HPLC) and FIG. 12 (IEX-HPLC).

For the conformational variant-enriched material the SE-HPLC and IEX-HPLC post peaks significantly decreased when the material was heated for 1 hour and 4 hours incubation at 50° C. (data for 4 hours incubation not shown). As this decrease was associated with an increase of the main peak for both analyses, this demonstrated that the conformational variant was converted to intact form. No change was observed for the conformational variant-depleted sample (data not shown).

Incubation at 60° C. seemed to be too high for the compound A and led to a degradation of the product with a decrease of the total area (loss of product) in SE-HPLC and IEX-HPLC (data not shown).

### Potency Recovering Upon pH or GuHCl Treatment

The potency towards TNFα (as described in Example 2) of compound A present in the conformational variant-enriched and -depleted fraction after pH 2.5 treatment for 4 hours or 2M GuHCl treatment for 0.5 hours was determined in comparison to untreated samples. The results are presented in Table 5.

TABLE 5
Potency results during analytical characterization.

| | | TNFα |
|---|---|---|
| Sample | Condition | Potency |
| “Depleted - Control” | Untreated conformational variant | 0.908 |
| | depleted fraction | |
| “Depleted - pH 2.5” | pH treated conformational variant | 0.929 |
| | depleted fraction | |
| “Depleted - GuHCl 2M” | GuHCl treated conformational | 0.441 |
| | variant depleted fraction | |
| “Enriched - Control” | Untreated conformational variant | 0.137 |
| | enriched fraction | |
| “Enriched - pH 2.5” | pH treated conformational variant | 0.887 |
| | enriched fraction | |
| “Enriched - GuHCl 2M” | GuHCl conformational variant | 0.547 |
| | enriched variant fraction | |

The potency drop for the untreated conformational variant enriched fraction compared to the conformational variant depleted fraction was confirmed. Low pH treatment of the conformational variant enriched fraction resulted in the regain of TNFα-potency to a level as observed for the conformational variant-depleted fraction. For the GuHCl treated samples, potency was lower but comparable for the enriched and depleted fractions after treatment.

### Summary

Altogether these experiments confirmed the presence of a conformational variant that can be converted to intact form under certain mild denaturing conditions or when modifying electrostatic interactions (pH). It was also shown that the conversion of the conformational variant into the intact ISVD product was maintained after removal of denaturing conditions or pH adjustment. Moreover, potency after conversion recovered and was maintained for 2 weeks at 25° C. or 5° C. (data not shown).

### 6.4 Example 4: Separation of the Conformational Variant of Compound a by Protein a Affinity Chromatography

### Use of Alternative Elution Buffers During Protein a Affinity Chromatography or Removal of Conformational Variant of Compound A

Based on the results obtained during characterization of the conformational variant (Examples 1 and 2), alternative elution buffer conditions were tested during capture of compound A.

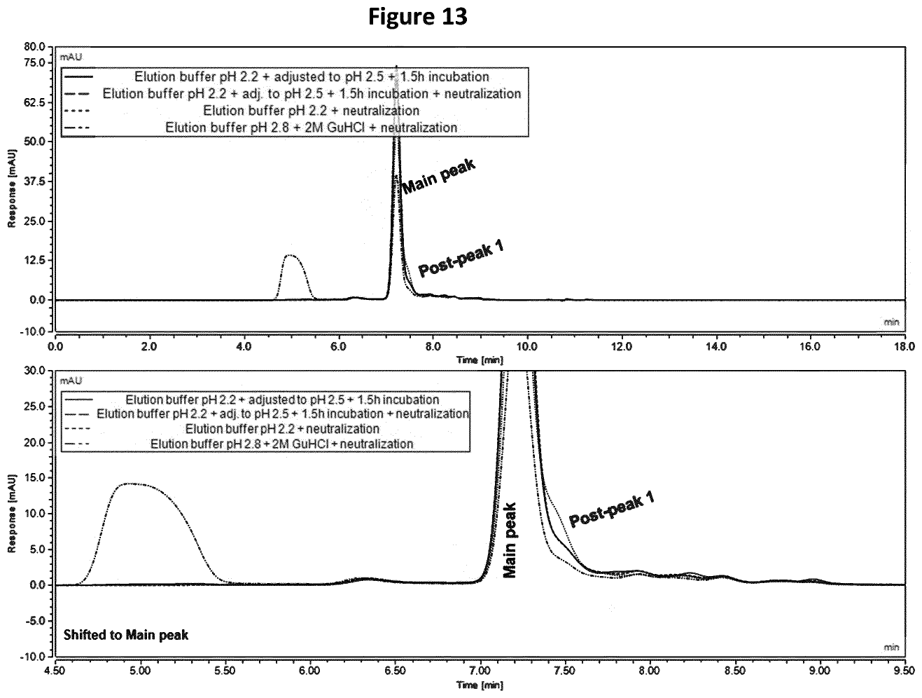

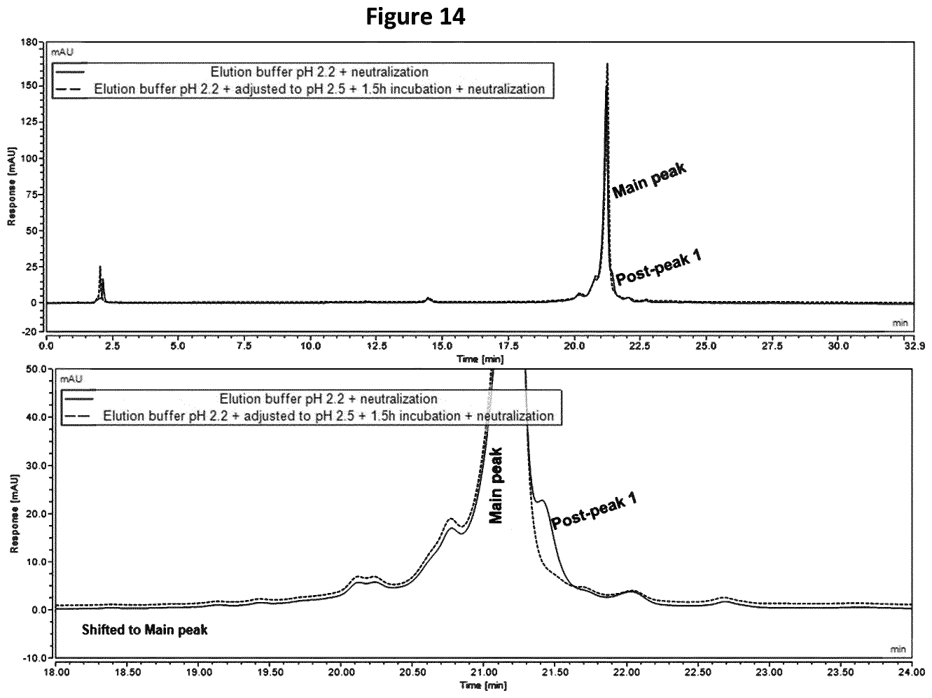

The elution conditions and results are presented in Table 6, FIG. 13 (SE-HPLC) and FIG. 14 (IEX-HPLC) (conditions as set forth in table C, SE-HPLC and IEX-HPLC protocol I).

TABLE 6
Capture conditions.

| | | Number CV/time/ | | Flow rate |
|---|---|---|---|---|
| Step | Buffer | loading factor | | (cm/h) |
| Equilibration | PBS | 8 | CV | 300 |
| Load | NA | 20 | g/L | 300 |
| Wash | Wash buffer | | | 300 |
| Elution | 100 mM glycine pH 2.2, | 5 | CV | 300 |
| | OR | | | |
| | 100 mM glycine pH 2.8 + | | | |
| | 2M GuHCl | | | |
| CIP | 0.1M NaOH | 15 | min | 300 |
| Re- | PBS | 8 | CV | 300 |
| equilibration | | | | |

The pH adjustment of the eluate to a pH of at least 7.0 was performed using 0.1 M NaOH.

For the run performed using the elution buffer at pH 2.2 in 0.1 M Glycine, part of the eluate material was directly adjusted to pH 7.1 using 0.1 M NaOH and for another part of the eluate material, the pH was adjusted to pH 2.5, incubated for 1.5 h and then the pH was readjusted to pH 7.0 with 0.1 M NaOH.

In SE-HPLC, it was seen that the post peak 1 was significantly reduced for elution buffer with GuHCl. However, the presence of GuHCl led to the degradation of the product as demonstrated by the formation of HMW species (pre-peak in SE-HPLC) in the eluate compared to the elution using a buffer at pH 2.2. For the elution at pH 2.2, the SE-HPLC post peak 1 was higher when the eluate was directly neutralized compared to the non-neutralized eluate or the eluate adjusted at pH 2.5 and incubated 1.5 h before neutralization (FIG. 13). This was confirmed on IEX-HPLC where the post peak shoulder disappeared for the eluate adjusted to pH 2.5 and incubated before neutralization compared to the eluate directly neutralized (FIG. 14).

For both analyses, the decrease of the post peak (conformational variant) was associated with an increase of the main peak (intact form). Altogether these results hinted for a conversion of the compact variant into the intact form.

Use of Low pH Incubation after Protein a Affinity Chromatography for Conversion of the Conformational Variant of Compound A

Based on the results obtained above, a low pH treatment as a mean of converting the conformational variant was investigated for compound A.

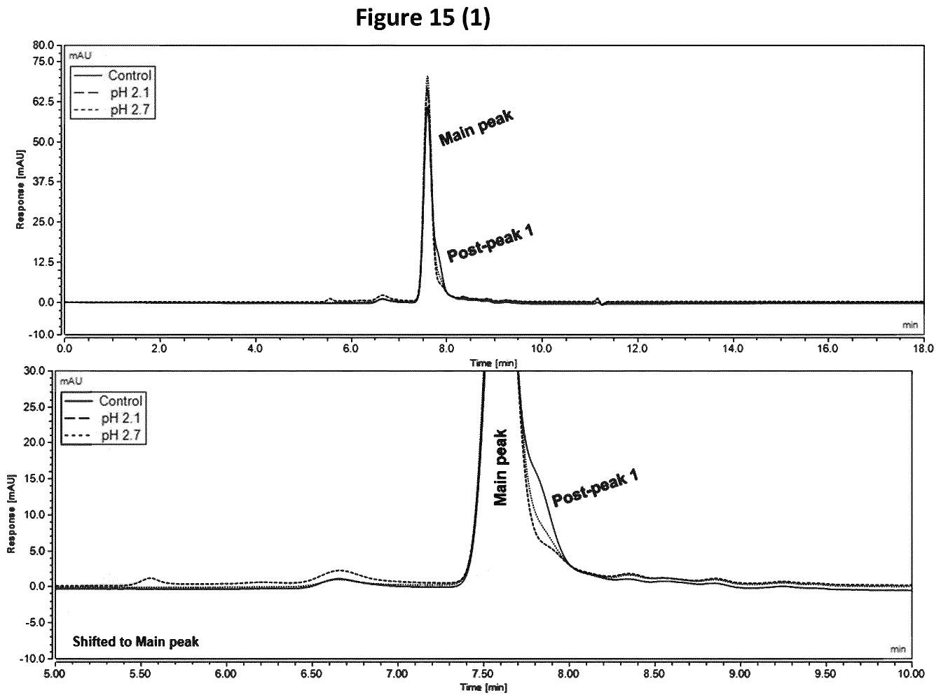

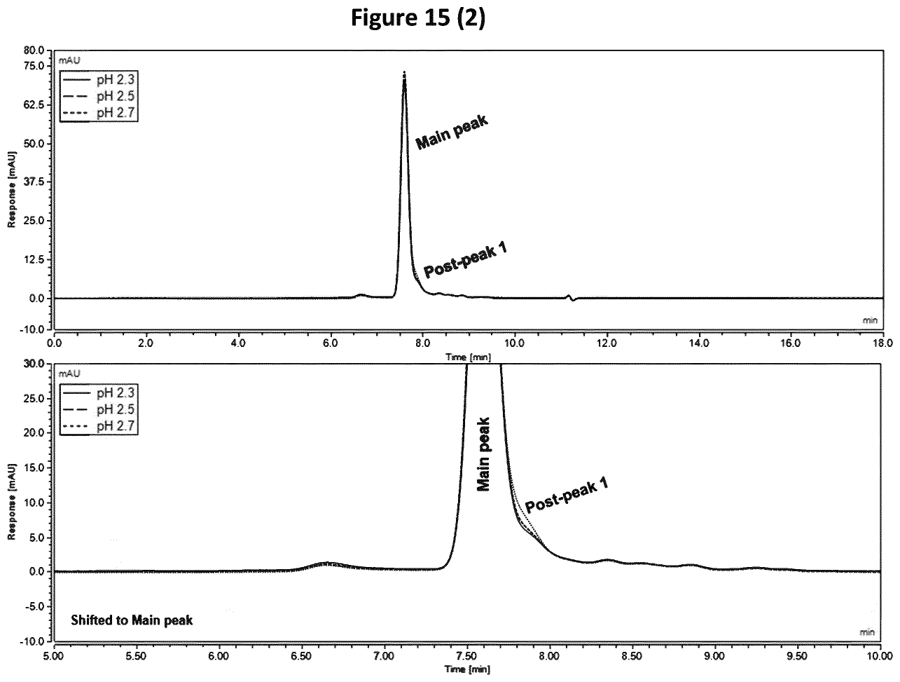

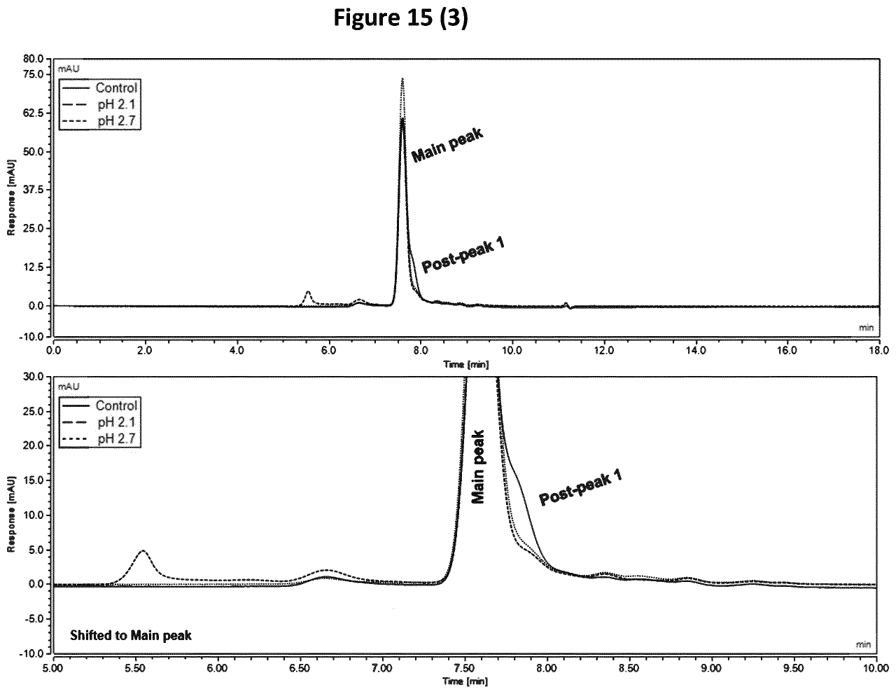

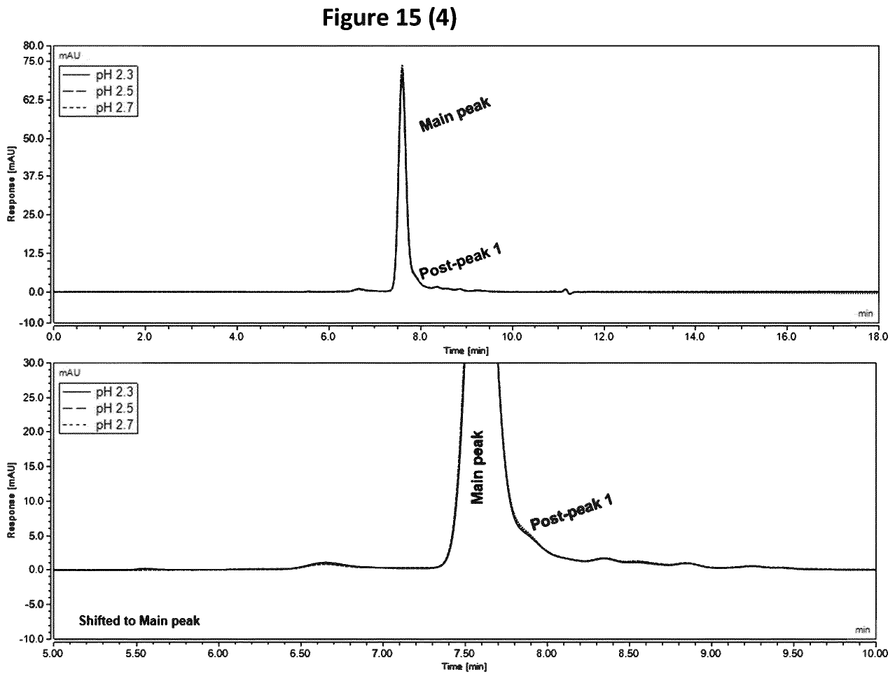

The impact of low pH treatment and length of incubation was investigated at pH 2.1, pH 2.3, pH 2.5 and pH 2.7 and incubations of 0, 1, 2, 4, 6 and 24 h. The pH of the capture eluate was decreased to the appropriate pH (2.1, 2.3, 2.5 or 2.7) with 0.1M HCl and was directly adjusted to pH 6.0 with 0.1M NaOH (T0) or incubated for 1 h or 2 h or 4 h or 6 h or 24 h at low pH and then adjusted to pH 6.0 with 0.1M NaOH (T1 h, T2 h, T4 h, T6 h, or T24 h). The product quality of the different low pH treated samples was compared to the capture eluate directly adjusted to pH 6.0 with 0.1M NaOH (control; T0) and analysed by IEX-HPLC, SE-HPLC, RP-UHPLC and capillary gel electrophoreses (CGE) (IEX-HPLC conditions as set forth in table C, protocol I). The SE-HPLC results are presented in FIGS. 15(1) and (2) (for T0) and FIGS. 15(3) and (4) (for T1 h).

At T0, the observed post-peak in SE-HPLC was lower at pH 2.1 and pH 2.7 compared to the control. This demonstrates that, at this pH range, the conversion of the conformational variant of compound A occurred instantaneously. However, the observed post peak 1 in SE-HPLC was lower for pH 2.1, 2.3, and 2.5 at T0 already meaning that the conversion of the conformational variant occurs instantaneously for pH equal to or lower than pH 2.5. This was confirmed in IEX-HPLC (data not shown) for which the post peak was lower for pH 2.3 and 2.5 compared to pH 2.7 at T0.

From an 1 h incubation onwards, the post peak shoulder in SE-HPLC was similar for all pH treatments.

Hence, the above data showed that the conversion of the conformational variant into intact form of the compound A is effective for all treatments at pH ranging from pH 2.1 to 2.7 for at least 1 h.

No changes were observed in RP-UHPLC and CGE (data not shown) indicating that the compact variant does not differ in molecular weight (no LMWs), chemical composition, or disulfide-bridging (scrambled S-S).

### Low pH Incubation for Conversion of Conformational Variant of Compound a is Independent of the Concentration of the pH Adjustment Stock Solutions

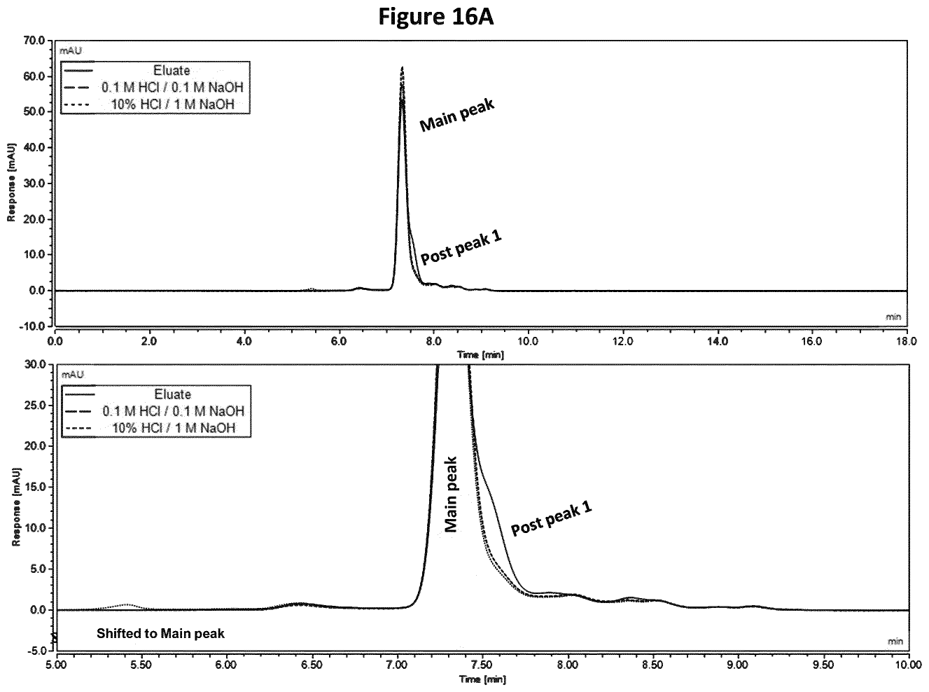

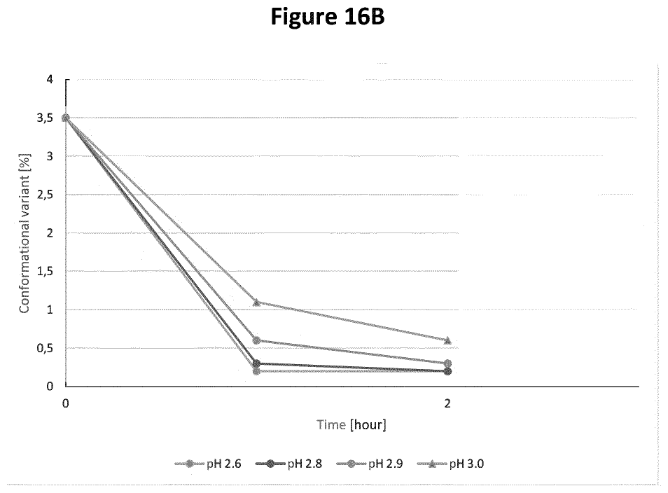

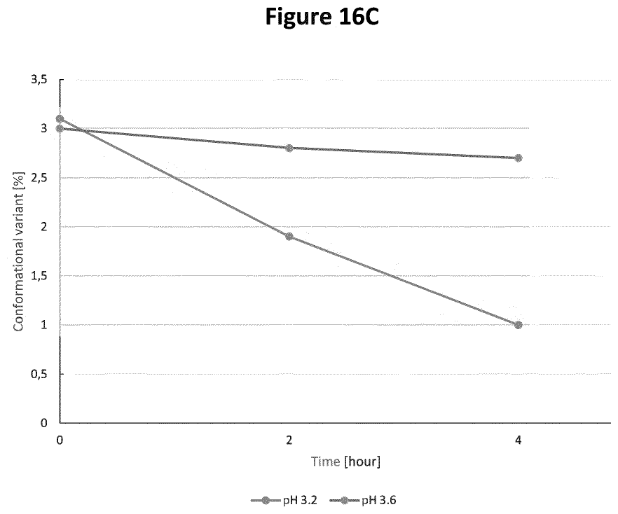

In order to investigate the influence of the concentration of the pH adjustment solutions, two sets of pH adjustment solutions were tested: the first one with 0.1 M HCl to decrease the pH to 2.6 and 0.1 M NaOH to adjust the pH to 6.0 and the second one with 2.7 M HCl (equals 10% HCl) to decrease the pH to 2.6 and 1M NaOH to adjust the pH to 6.0. Samples were incubated for 1 h at pH 2.6 before adjustment to pH 6.0. The SE-HPLC results are presented in FIG. 16A.

The use of the two sets of pH adjustment solutions led to comparable results with a decrease of the SE-HPLC post peak associated with an increase of the main peak related to the conversion of the conformational variant into intact form.

The low pH incubation step was then introduced in the process for intermediate scale runs to assess intermediate scalability. The pH was decreased to pH 2.6 using 0.1M HCl and then adjusted to pH 6.0 after 1 h by adding 0.1 M NaOH.

SE-HPLC (conditions as set forth in table C) results showed a decrease of the post peak 1 associated with an increase of the main peak for the capture filtrate (with low pH incubation) compared to the capture eluate (before low pH treatment) confirming the conversion of the conformational variant into intact form (data not shown).

### Influence of Other Low pH Treatments on Conformational Variant of Compound A

After expression of compound A in P. pastoris and clarification with tangential flow filtration, a capture chromatography using Amsphere A3 resin was used to isolate compound A from other impurities.

The column was first equilibrated with PBS buffer pH 7.5 and loaded with clarified cell-free harvest material containing the compound of interest. Compound A binds to the Amsphere A3 resin and impurities flow through the column. Subsequently, the loaded resin was washed with the same PBS buffer as the equilibration step, followed by tris buffer to wash. The tris buffer contained 100 mM tris and 1M NaCl at pH 8.5. The resin was further washed with a second 100 mM Tris buffer at pH 5.5. Compound A was eluted from the column with a low pH glycine buffer. The low pH glycine elution buffer contained 100 mM Glycine at pH 3.0. Finally, the resin was cleaned with 100 mM NaOH before storage in the same PBS buffer as equilibration. All buffers were run at 183 cm/h.

In a first experiment, the pH of the capture eluate material of compound A was decreased to pH 2.6, pH 2.8, pH 2.9, and pH 3.0 with 1 M HCl. After a 1 h and 2 h of incubation at low pH, samples were adjusted to pH 6.0 with 0.2M NaOH. The T0 sample, or control sample, was the capture chromatography that was immediately frozen after elution. This sample had a pH 4.3.

In a second experiment, the pH of the product eluting from the chromatography column was 4.1 and 3.7 in two capture chromatography runs. The pH of the capture eluate was decreased to pH 3.2 or pH 3.6 with 1M HCl. After a 2 h and 4 h of incubation at low pH, samples were adjusted to pH 6.0 with 0.2M NaOH. The T0 was generated by decreasing compound A to the target low pH (i.e pH 3.2 or 3.6) with 1M HCl and directly adjusted to pH 6.0 with 0.2M NaOH (T0).

The influence of pH on the product quality was analyzed in function of time by IEX-HPLC. See Tables 6-1 and 6-2 and FIGS. 16B and C.

TABLE 6-1
Results of IEX-HPLC analysis of the low pH treatment impact on
conversion of the conformational variant (first experiment).

| | Time point | IEX-HPLC |
|---|---|---|
| pH | [hour] | Post-peak 1 (%) |
| Control | 0 | 3.5 |
| 2.6 | 1 | 0.2 |
| 2.6 | 2 | 0.2 |
| 2.8 | 1 | 0.3 |
| 2.8 | 2 | 0.2 |
| 2.9 | 1 | 0.6 |
| 2.9 | 2 | 0.3 |
| 3.0 | 1 | 1.1 |
| 3.0 | 2 | 0.6 |

TABLE 6-2
Results of IEX-HPLC analysis of the low pH treatment impact on
conversion of the conformational variant (second experiment).

| | Time point | IEX-HPLC |
|---|---|---|
| pH | [hour] | Post-peak 1 (%) |
| 3.2 | 0 | 3.1 |
| 3.2 | 2 | 1.9 |
| 3.2 | 4 | 1.0 |
| 3.6 | 0 | 3.0 |
| 3.6 | 2 | 2.8 |
| 3.6 | 4 | 2.7 |

The IEX-HPLC results show the positive influence of a low pH treatment over time on the presence of conformational variants in the sample. In the first set of experiments (pH 2.6, pH 2.8, pH 2.9 and pH 3.0), the level of conformational variant in the control samples was 3.5%. In the second set of experiments (pH 3.2 and 3.6), the level of conformational variant was 3.1%.

After 2 h of incubation at low pH, the level of conformational variant decreased in all pHs tested. The positive effect of low pH treatment on the conformational variant increased with lower pHs. The best reduction was observed between pH 3.0 and pH 2.6.

### 6.5 Example 5: Scale Up of the Low pH Treatment of Compound a (10 L and 100 L)

Based on the preceding Examples, the conditions selected for the low pH incubation of compound A were a target pH of 2.6 for ≥60 and ≤120 min at room temperature. The pH of the capture eluate was lowered using 0.1M HCl and then adjusted to pH 6.0 after 60 and 120 min by adding 0.1 M NaOH. The fermentation process was scaled up to scales of 10 L and 100 L. The product quality of the capture eluate before low pH treatment (referred to as “capture eluate”) and of the capture eluate after low pH treatment followed by pH adjustment to 6.0 as described above and filtration (referred to as capture filtrate) was determined by analytical methods such as SE-HPLC, CGE and IEX-HPLC (conditions as set forth in table C, IEX-HPLC protocol I). To process all the starting material, 3 cycles of capture step were performed for each scale. The results for the different scales are presented in Table 7.

TABLE 7
Impact of the low pH treatment on the product quality
of compound A during scaling up.

| | | | | | SE-HPLC | CGE |
|---|---|---|---|---|---|---|
| | | | IEX-HPLC | IEX-HPLC | HMW | Main |
| | | | Main peak | Post-peak* | species | peak |
| Scale | Cycles | | (%) | (%) | (%) | (%) |
| 10 L | Cyclel | eluate | 74.5 | 21.1 | 4.4 | 84.4 |
| | | filtrate | 86.4 | 8.4 | 2.0 | 84.2 |
| | Cycle 2 | eluate | 75.1 | 20.2 | 4.0 | 82.8 |
| | | filtrate | 86.9 | 7.9 | 1.8 | 83.8 |
| | Cycle 3 | eluate | 75.5 | 19.4 | 4.0 | 83.2 |
| | | filtrate | 87.5 | 7.5 | 2.0 | 82.6 |
| 100 L | Cyclel | eluate | 72.5 | 17.5 | 2.7 | 82.6 |
| | | filtrate | 80.4 | 9.5 | 1.8 | 82.8 |
| | Cycle 2 | eluate | 72.5 | 17.0 | 2.9 | 82.5 |
| | | filtrate | 79.9 | 9.4 | 1.7 | 82.7 |
| | Cycle 3 | eluate | 71.9 | 16.9 | 2.9 | 82.4 |
| | | filtrate | 79.3 | 9.4 | 1.8 | 82.3 |

*Sum of all postpeaks

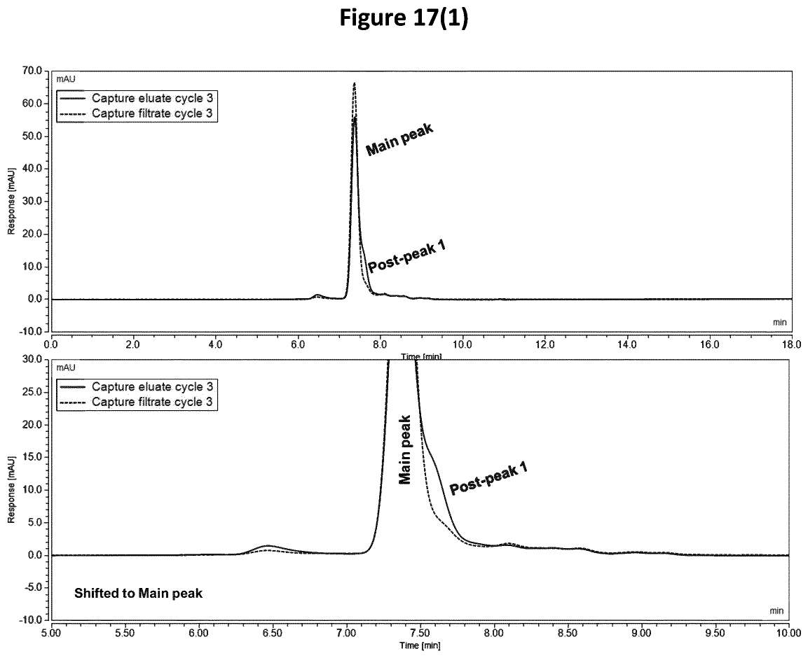

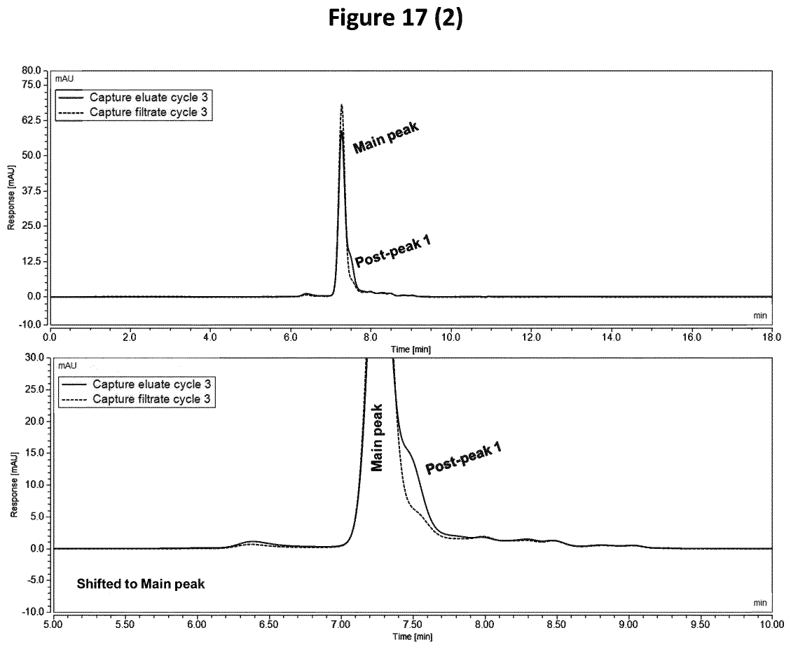

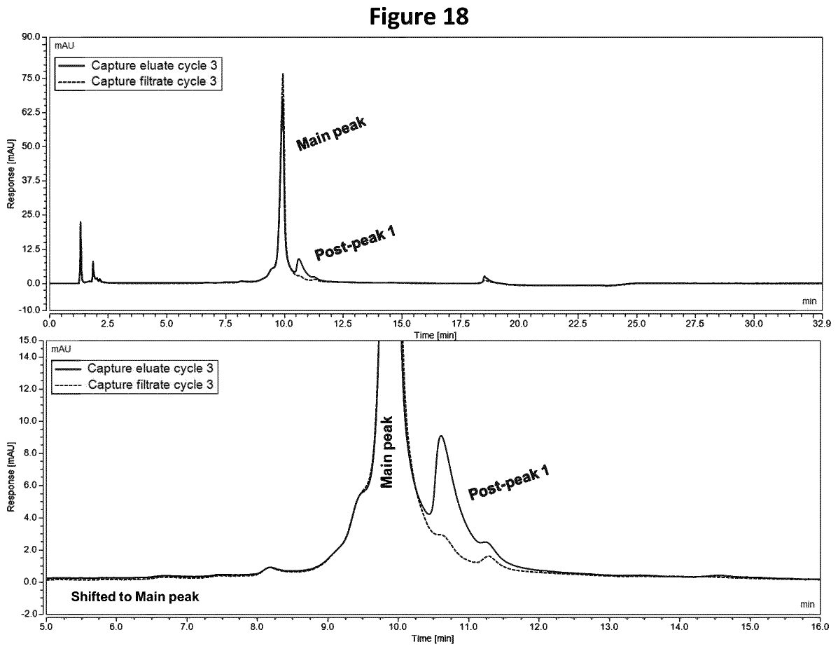

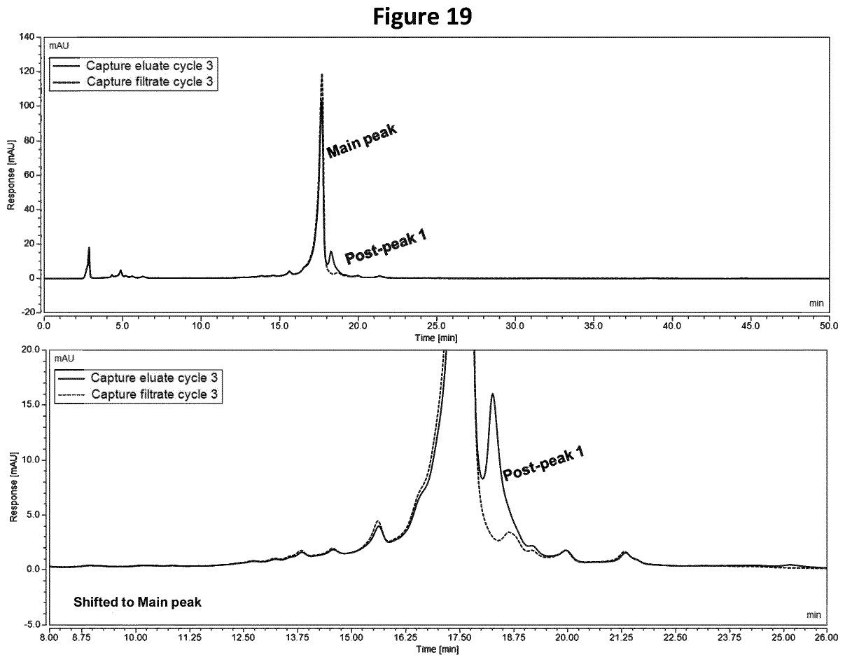

Independently of the fermentation and purification scales, the low pH treatment and filtration step had no impact on the product purity with regard to % main peak on CGE analysis. The results were within method variability. However, surprisingly, a decrease in % HMW species by SE-HPLC (see FIG. 17(1) (10 L) and FIG. 17(2) (100 L)) was observed in both fermentation (10 L and 100 L, respectively) and purification scale-up (7 cm and 20 cm column diameter, respectively) when comparing the capture filtrates and the capture eluates; this decrease being the result of the low pH treatment and/or the filtration step. Moreover, as observed before at small-scale, a significant increase in % main peak purity as well as a decrease in % post-peak (conformational variant) was observed on IEX-HPLC after low pH treatment when comparing capture filtrates with capture eluates (Table 7 and FIGS. 18 (10 L) and 19 (100 L)). Further, a decrease of the post peak 1 (shoulder) was associated with an increase of the main peak for the profiles of the capture filtrates. This correlates with the IEX data and confirms the conversion of the conformational variant into intact form of compound A.

Altogether, the results showed that the low pH treatment was a scalable process and was efficient to convert conformational variant into intact form of compound A.

### 6.6 Example 6: Separation of Conformational Variants of the Compound a by Other Chromatographic Techniques

### Use of Mixed Mode Chromatography (MMC) for Removal of Conformational Variant of Compound A

In the above Examples it could be demonstrated that the conformational variant of compound A can be reliably separated using IEX-based chromatography methods. In order to determine if the removal of the less potent conformational variant from a mixture of conformational variant and intact form can be also achieved by other chromatographic methods, a mixed-mode chromatography (MMC) using a CHT ceramic hydroxyapatite type II (40 μm) resin (BioRad) was performed. The chromatographic conditions are summarized in Table 8.

TABLE 8
Conditions for gradient on hydroxyapatite resin for the removal
of compound A conformational variant.
Buffers

| | |
|---|---|
| Equilibration | 10 mM Sodium Phosphate pH 6.5 + |
| | 10 ppm Ca²⁺ − 2 CV |
| Wash | 10 mM Sodium Phosphate pH 6.5 + |
| | 10 ppm Ca²⁺ − 1 CV |
| Elution | 200 mM Sodium Phosphate pH 6.5 + |
| | 10 ppm Ca²⁺ − Gradient 0-70% − 20 CV |
| Regeneration | 400 mM Sodium Phosphate pH 6.5 + |
| | 10 ppm Ca²⁺ − 2 CV |
| Cleaning in place (CIP) | 1M NaOH − 2 CV |
| Storage | 100 mM NaOH − 5 CV |

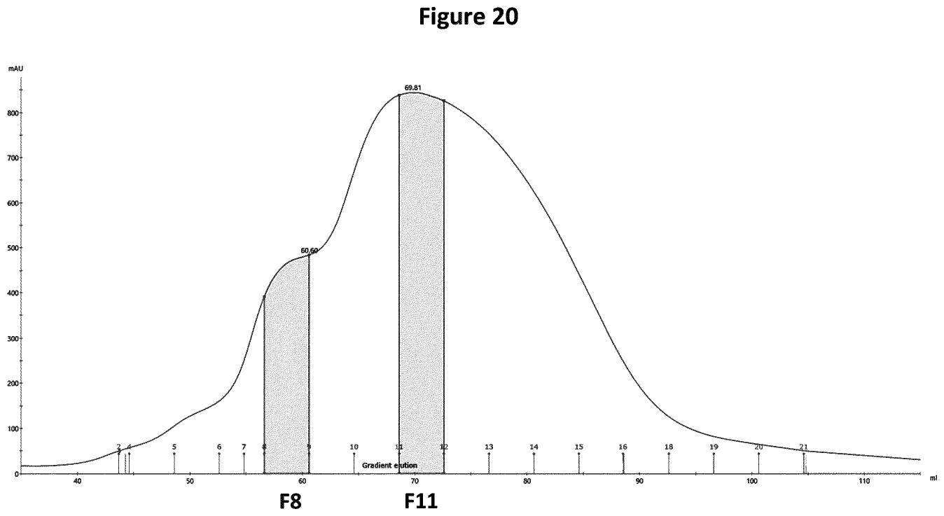

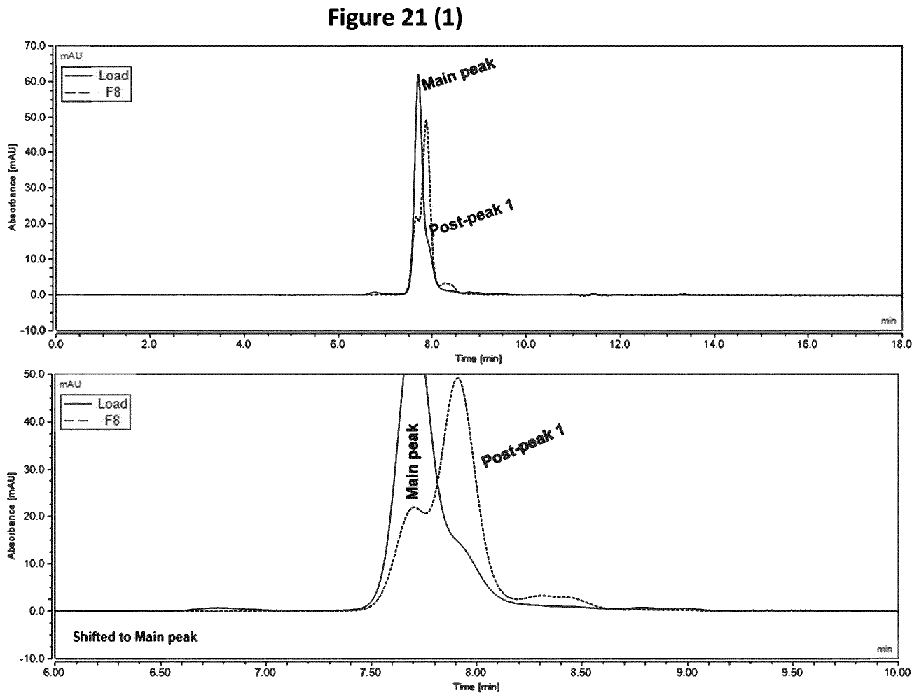

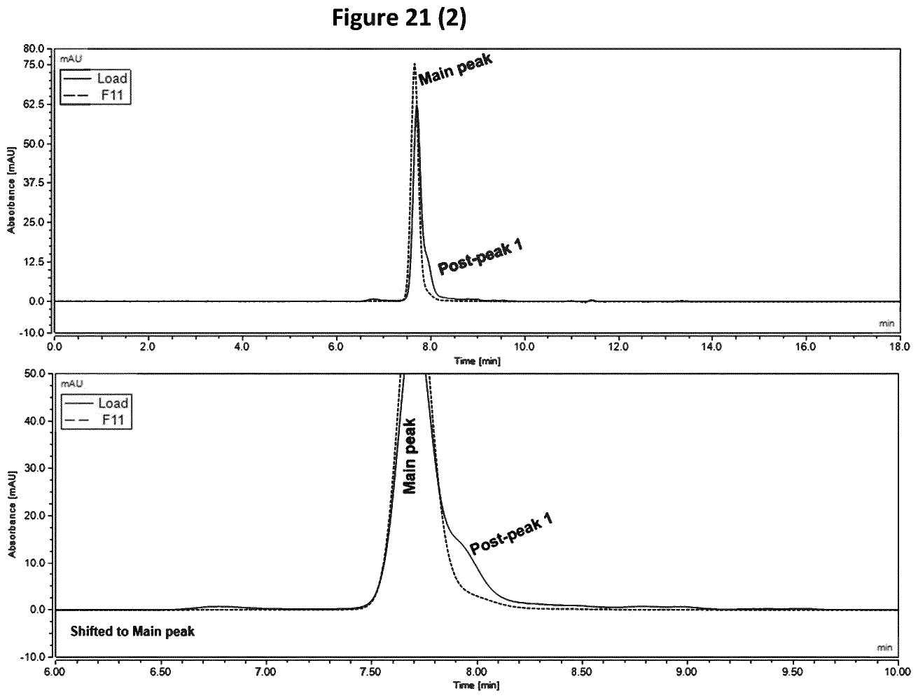

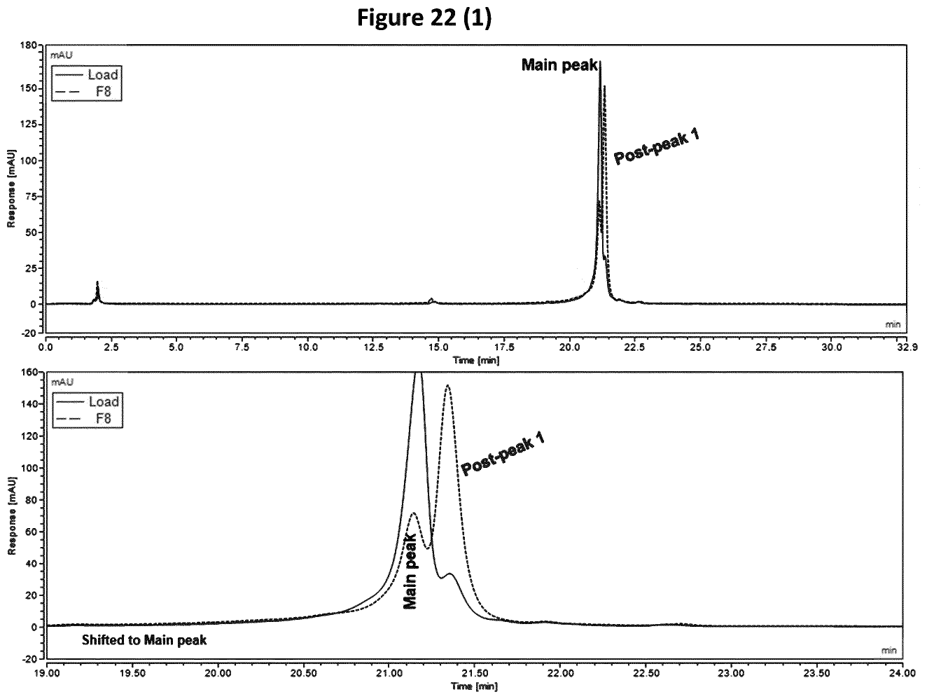

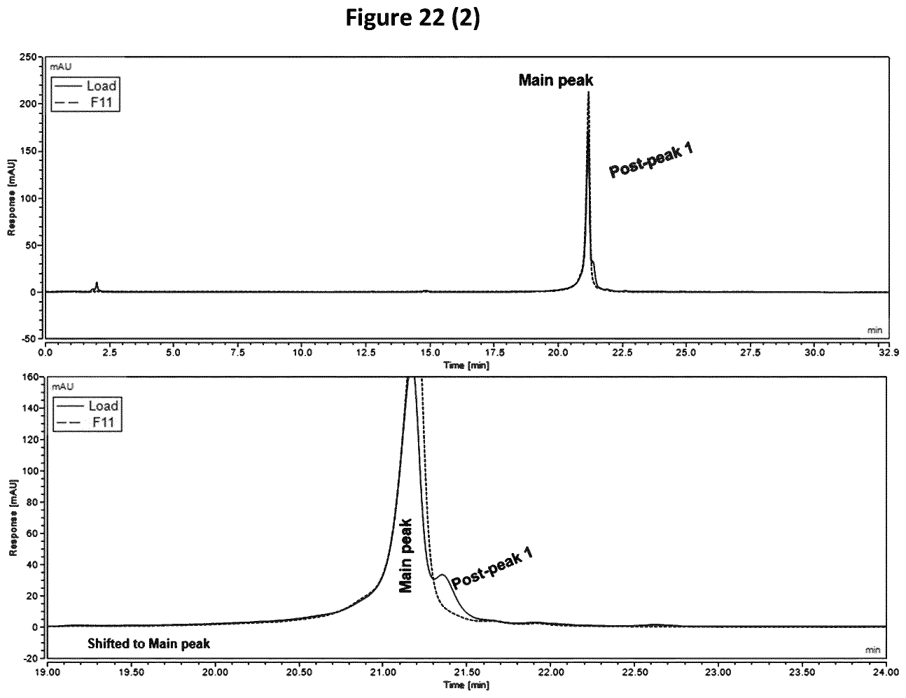

The chromatographic profile on hydroxyapatite resin is shown in FIG. 20. Similar to the CEX, a side (front) fraction (F8) and top fraction (F11) were isolated and used for further SE-HPLC and IEX-HPLC analysis. The results of both analyses are shown in FIG. 21(1)/(2) (SE-HPLC) and FIG. 22(1)/(2) (IEX-HPLC). A significant post peak 1 on SE-HPLC and IEX-HPLC (conditions as set forth in Table C; IEX-HPLC protocol I) was observed for the fraction F8 (side fraction taken from the peak before the main/top peak) demonstrating that this fraction was enriched for the conformational variant. Fraction F11 was depleted from the conformational variant as, for this fraction F11, the SE-HPLC and IEX-HPLC post peak 1 was significantly reduced compared to the load material.

In conclusion, the results on hydroxyapatite resin were similar to the results obtained with cation exchange resin. Thus, hydroxyapatite resin was shown to be suitable for the removal of the less potent conformational variant from a mixture of both conformational variant and intact form of compound A.

### Use of Hydrophobic Interaction Chromatography (HIC) for Removal of Conformational Variant of Compound A

As the separation of the compact conformational variant from the intact form of compound A was observed for different chromatographic techniques and types of resins, another chromatographic method, hydrophobic interaction chromatography (HIC), was tested. First, a gradient using HIC TSK Phenyl gel 5 PW(30) (Tosoh) resin was performed using the conditions presented in Table 9.

TABLE 9
Conditions for gradient on HIC TSK Phenyl gel 5 PW (30) resin
for the removal of F02730252 conformational variant.
Buffers

| | |
|---|---|
| Equilibration | 25 mM TRIS pH 7 + 1M (NH4)2SO4 − 2 CV |
| Wash | 25 mM TRIS pH 7 + 1M (NH4)2SO4 − 2 CV |
| Elution | 25 mM TRIS pH 7Gradient 0-100% − 30 CV |
| CIP | 0.5M NaOH − 2 CV |
| Storage | 10 mM NaOH − 3 CV |

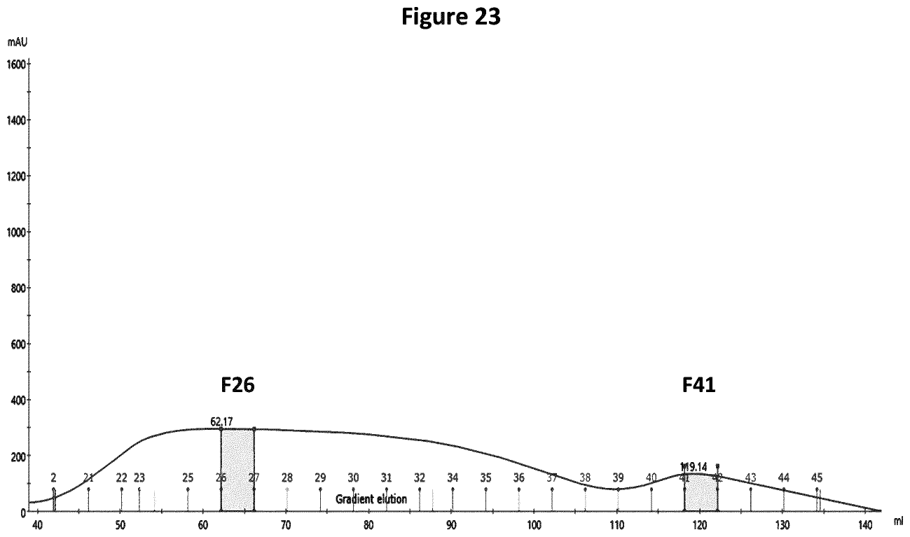

The corresponding HIC chromatogram is depicted in FIG. 23. As seen for the CEX and MMC chromatograms, the tested gradient resulted in an HIC profile with two separated peaks (1^{st} (main) peak also followed by a 2^{nd} (side) peak). One representative fraction of each peak was further analysed. The SE-HPLC data (conditions as set forth in Table C) from the selected fractions of the main peak (F26; top fraction) and the side peak (F41; side fraction) are presented in FIG. 24(1)/(2). The corresponding SE-HPLC profiles revealed that the top fraction is constituted only of the earlier eluting intact form as no post peak 1 is seen on SE-HPLC. In contrast, the SE-HPLC data showed that the main species of the side fraction is almost entirely the later eluting conformational variant (almost 100% post peak 1).

Hence, using a gradient on HIC, a good separation of conformational variant from the desired intact form could be achieved. Accordingly, this HIC resin was shown to be suitable for the removal of conformational variant of a mixture of both the conformational variant and the intact form of compound A.

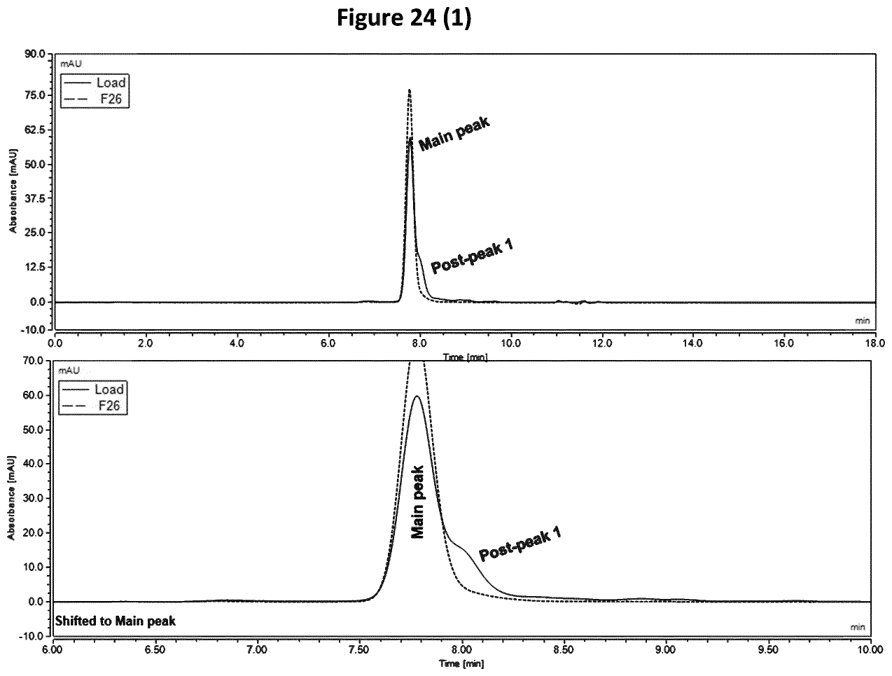

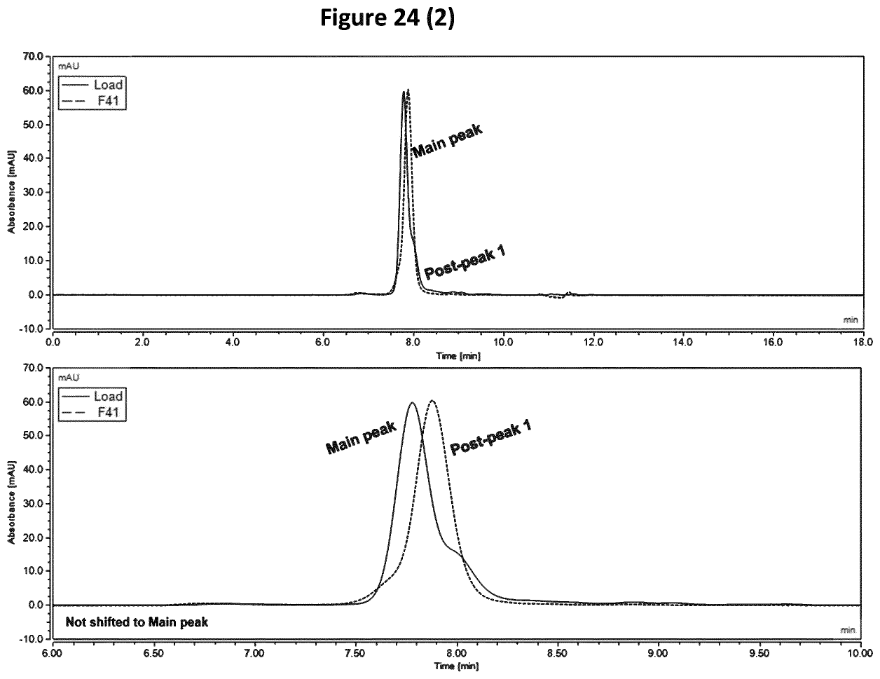

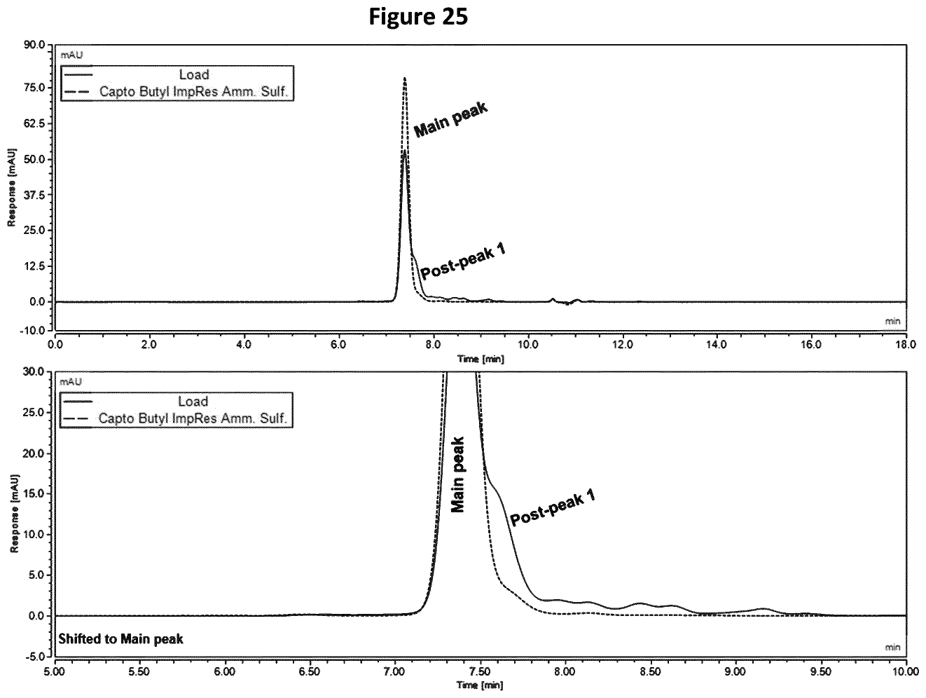

Since the initially tested HIC resin (TSK Phenyl gel 5 PW(30) resin) was a high resolution resin, other HIC resins that are more suitable for large scale processing were tested: Capto phenyl High Sub (GE Healthcare), Capto phenyl ImpRes (GE Healthcare), Capto butyl ImpRes (GE Healthcare), Phenyl HP (GE Healthcare) and Capto Butyl (GE Healthcare). Gradients using ammonium sulfate and sodium chloride were tested. The conditions used are described in Table 10 below. The SE-HPLC profile of the top fraction and load for the resin Capto Butyl Impres used with an ammonium sulfate gradient is presented in FIG. 25.

TABLE 10
Conditions for gradient on Capto phenyl High Sub, Capto phenyl
ImpRes, Capto butyl ImpRes, Phenyl HP, Capto Butyl ImpRes and
Capto butyl resins for the removal of the compound A
conformational variant.
Buffers

| | |
|---|---|
| Equilibration | 50 mM Phosphate pH 6.0 + 1M (NH4)2SO4 |
| | 50 mM Phosphate pH 6.0 + 3M NaCl (Phenyl HP and |
| | Capto butyl ImpRes) −3 CV |
| Wash | 50 mM Phosphate pH 6 + 1M (NH4)2SO4 |
| | 50 mM Phosphate pH 6.0 + 3M NaCl (Phenyl HP and |
| | Capto butyl ImpRes) −3 CV |
| Elution 1 | 50 mM Phosphate pH 6.0 0-100% 30 CV |
| Elution 2 | 50 mM Phosphate pH 6.0 100% 27 CV (capto phenyl |
| (regeneration) | high sub in (NH₄)₂SO₄) |
| | 50 mM Phosphate pH 6.0 100% 13 CV (capto phenyl |
| | ImpRes in (NH₄)₂SO₄) |
| | 50 mM Phosphate pH 6.0 100% 7 CV (capto butyl |
| | ImpRes in (NH₄)₂SO₄) |
| | 50 mM Phosphate pH 6.0 100% 2 CV (Phenyl HP in |
| | ((NH₄)₂SO₄) |
| | 50 mM Phosphate pH 6.0 100% 5 CV (capto butyl in |
| | (NH₄)₂SO₄) |
| | 50 mM Phosphate pH 6.0 100% 10 CV (capto butyl |
| | ImpRes in NaCl) |
| | 50 mM Phosphate pH 6.0 100% 2 CV (Phenyl HP in |
| | NaCl) |
| CIP | 0.5M NaOH |
| Storage | 10 mM NaOH |

The post peak in SE-HPLC was significantly reduced for all the tested resins, both for gradients using sodium chloride and ammonium sulfate, with the exception of Capto Phenyl High sub. It was consequently confirmed that the conformational variant can be removed using process suitable HIC resins with either sodium chloride or ammonium sulfate gradients.

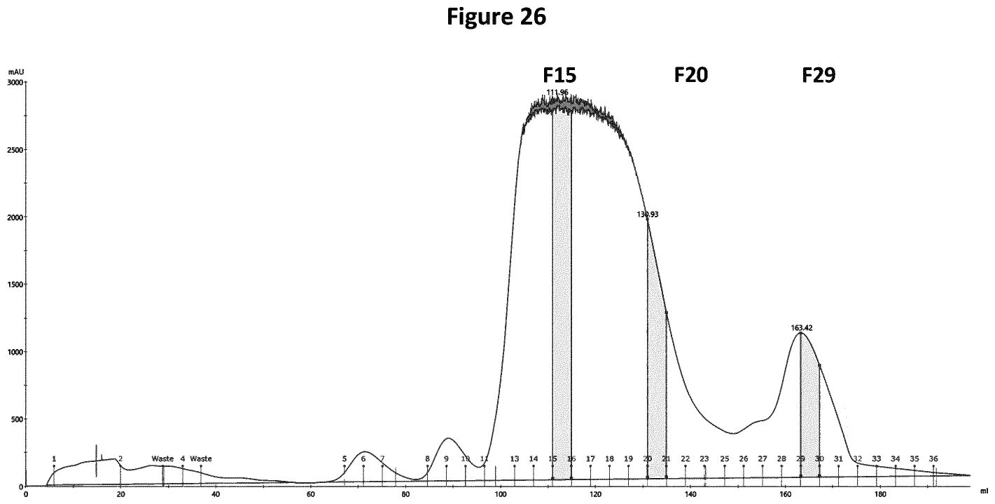

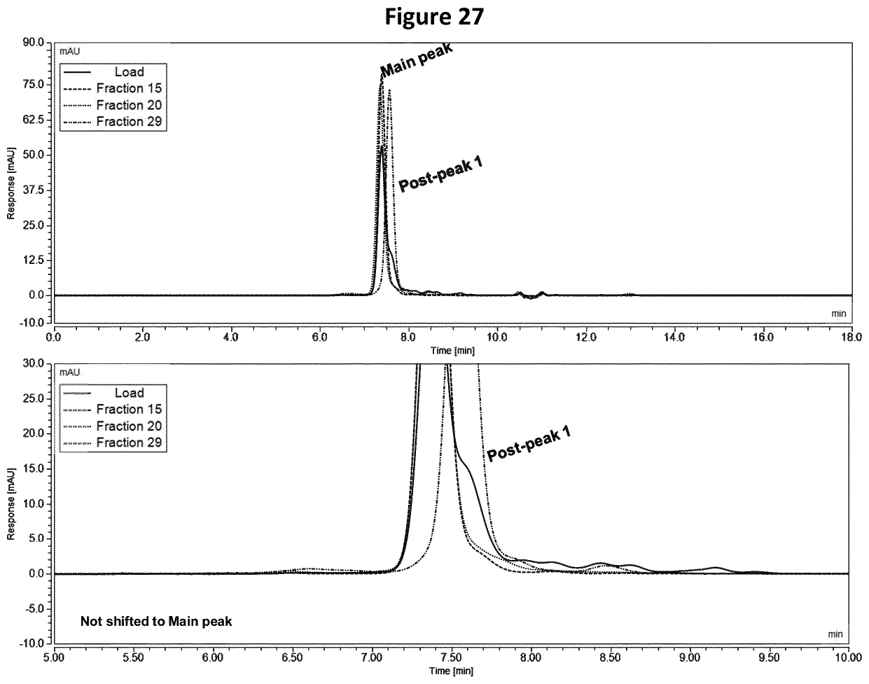

The HIC chromatogram of the resin Capto Butyl Impres and an ammonium sulfate gradient is presented in FIG. 26. As seen on the chromatogram, the tested gradient led to two separated peaks, a 1^{st} (main) peak followed by a smaller 2^{nd} (side) peak. Several fractions of the main peak (F15 and F20) and one fraction of the 2^{nd} (side) peak (F29) were further analysed by SE-HPLC. The resulting chromatograms (FIG. 27) demonstrated that fraction F29 exclusively contained the later eluting conformational variant (almost 100% SE-HPLC post peak 1; see peak shift compared to the load peak). In contrast, fractions 15 and 20 of the main peak did not show the presence of the SE-HPLC post peak 1 demonstrating that these fractions are depleted for the later eluting, undesired conformational variant.

Accordingly, using Capto Butyl Impres resin, a good separation of the conformational variant of compound A using a gradient on hydrophobic interaction was achieved. Thus, this resin was shown to be usable for the removal of the conformational variant from a mixture of both conformational variant and intact form of compound A.

### Use of Membrane-Based HIC for Removal of Conformational Variant of Compound A

As the separation of the conformational variant and the intact form could be well achieved using HIC resin in columns, a step in flow-through mode with the desired intact form in the flow-through was developed. Therefore, an additional HIC setup using the HIC membrane Sartobind Phenyl (Sartorius) was performed. The conditions of screening on Sartobind phenyl membrane (filter plate) are described in Table 11.

TABLE 11
Screening conditions on Sartobind Phenyl membrane (filter plate)
for the removal of undesired conformational variant.
Conditions

| | |
|---|---|
| Buffers | Phosphate pH 6.0 and pH 7.0 |
| Salt | Ammonium Sulfate (700-50 mM) |
| | Sodium Sulfate (700-50 mM) |
| | Sodium Chloride (3000-400 mM) |
| Start material | Polish eluate |

| | 1 | 2 | 3 | 4 | 5 | 6 |
|---|---|---|---|---|---|---|
| | Ammonium Sulfate | | Sodium Phosphate | | Sodium Chloride | |
| | (mM) | | (mM) | | (mM) | |
| | | Sodium | | Sodium | | Sodium |
| | Sodium | Phos- | Sodium | Phos- | Sodium | Phos- |
| | Phosphate | phate | Phosphate | phate | Phosphate | phate |
| | pH 6.0 | pH 7.0 | pH 6.0 | pH 7.0 | pH 6.0 | pH 7.0 |
| A | 700 | 700 | 700 | 700 | 3000 | 3000 |
| B | 600 | 600 | 600 | 600 | 2500 | 2500 |
| C | 500 | 500 | 500 | 500 | 2000 | 2000 |
| D | 400 | 400 | 400 | 400 | 1500 | 1500 |
| E | 300 | 300 | 300 | 300 | 1000 | 1000 |
| F | 200 | 200 | 200 | 200 | 800 | 800 |
| G | 100 | 100 | 100 | 100 | 600 | 600 |
| H | 50 | 50 | 50 | 50 | 400 | 400 |

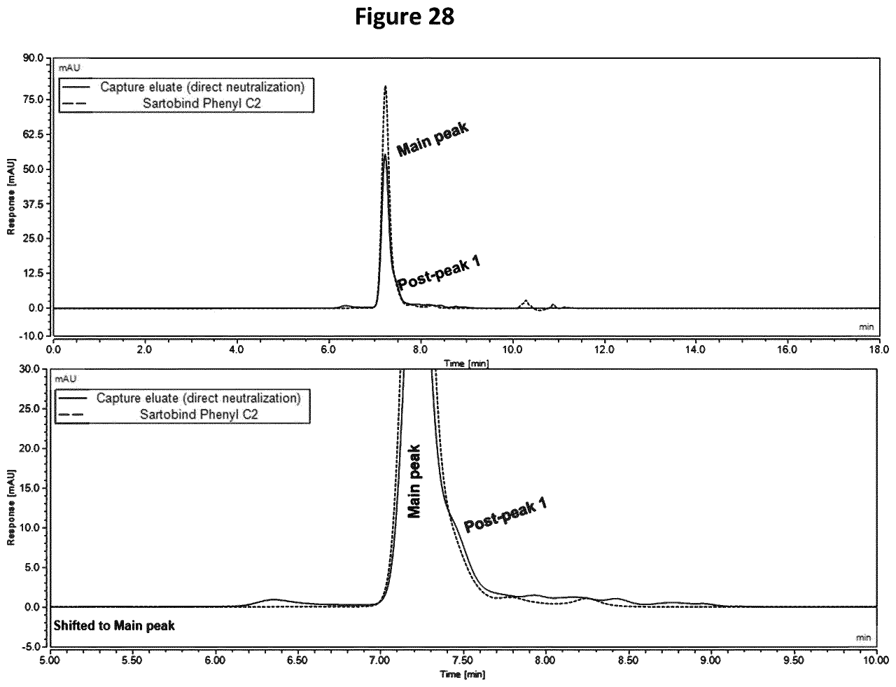

The SE-HPLC profile of a representative condition (condition C2) is presented in FIG. 28. The SE-HPLC post peak 1 was significantly reduced compared to a reference sample containing the conformational variant. As reference was used a capture eluate (from protein A-affinity chromatography) that did not undergo low pH treatment but was directly neutralized to pH 7.4. The reference was not subject to HIC. The conformational variant was thus neither removed nor converted from the reference sample.

Further optimization was performed using a 3 mL Sartobind phenyl membrane. The conditions are described in Table 12. Ammonium sulfate and sodium chloride were used at different concentrations to optimize recovery of the intact form in the flow-through.

TABLE 12
Screening conditions on Sartobind Phenyl membrane for
the removal of conformational variant of compound A.

| | | | Flow rate |
|---|---|---|---|
| Phase | Buffer | MV | (MV/min) |
| Venting | Equilibration buffer | 7 (2x) | NA |
| CIP | 1M NaOH | 30 | 1 |
| Flush | H2O | 25 | 5 |
| Equilibration | 50 mM Sodium Phosphate pH 6 + | 10 | 5 |
| | 461 mM Ammonium Sulfate/ | | |
| | 1080 mM Sodium Chloride | | |
| Load | CEX eluate diluted ½ with 100 mM | | 5 |
| | Sodium Phosphate pH 6.0 + 800 mM | | |
| | Ammonium Sulfate/2000 mM | | |
| | Sodium Chloride | | |
| Wash | 50 mM Sodium Phosphate pH 6 + | 20 | 5 |
| | 461 mM Ammonium Sulfate/ | | |
| | 1080 mM Sodium Chloride | | |
| Strip | 50 mM Sodium Phosphate pH 6.0 | 15 | 5 |
| Regeneration | 70% Ethanol | 10 | 5 |
| Storage | 20% EtOH | | 5 |

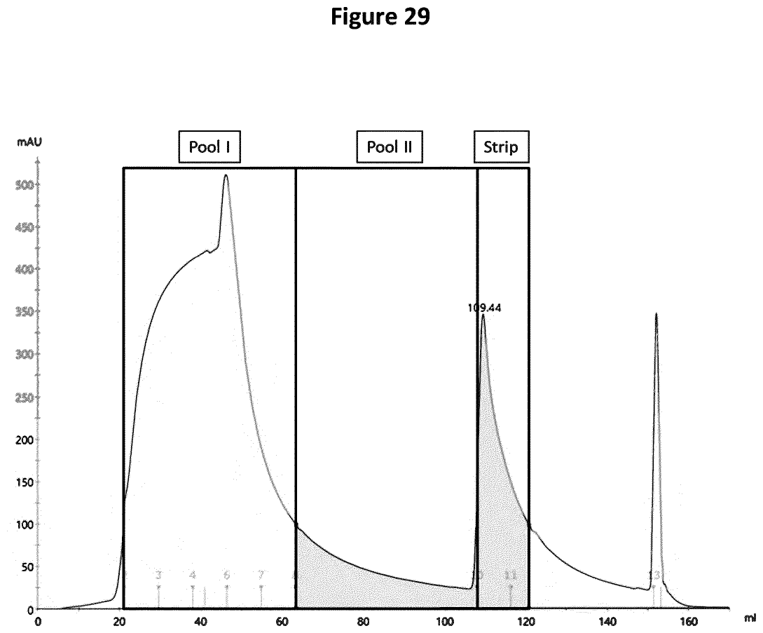

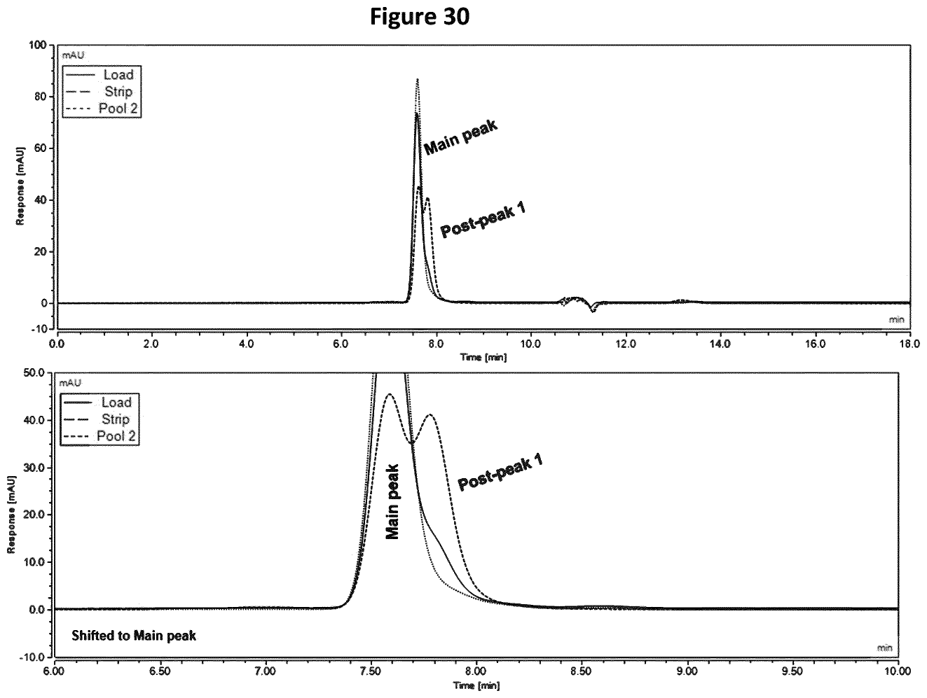

The HIC chromatogram for the optimal condition is presented in FIG. 29. The SE-HPLC data from the load, fraction pool 2, and strip fraction are shown in FIG. 30. The SE-HPLC post peak 1 was significantly reduced for pool 2 from the flow-through of the membrane. The strip was enriched in SE-HPLC post-peak shoulder i.e., undesired conformational variant. Accordingly, the conformational variant was removed from the desired intact form of compound A using HIC phenyl membrane in flow-through mode. The recovery was 74% using Ammonium Sulfate (pool 2) and 63% using Sodium Chloride (pool 2).

### 6.7 Example 7: Identification and Initial Characterization of a Compact Variant of Compound B

In order to confirm that a compact variant also appears for other multivalent ISVD constructs, further investigations were made for compound B.

Compound B (SEQ ID NO: 2) is a multivalent ISVD construct comprising four different sequence optimized variable domains of heavy-chain llama antibodies that bind to three different targets. The ISVD building blocks are fused head-to-tail (N-terminus to C-terminus) with a G/S linker in the following format: a TNFα-binding ISVD—9GS linker—an IL23p19-binding ISVD—9GS linker—a human serum albumin-binding ISVD—9GS linker—an IL23p19-binding ISVD and having the following sequence:

TABLE 13
Amino acid sequence of compound B.
Compound B (SEQ ID NO: 2)

DVQLVESGGGVVQPGGSLRLSCTASGFTFSTADMGWFRQAPGKGREFVARISGIDGTTYYDEPVKGRFT
ISRDNSKNTVYLQMNSLRPEDTALYYCRSPRYADQWSAYDYWGQGTLVTVSSGGGGSGGGSEVQLVES
GGGVVQPGGSLRLSCAASGRIFSLPASGNIFNLLTIAWYRQAPGKQRELVATIESGSRTNYADSVKGRFTI
SRDNSKKTVYLQMNSLRPEDTALYYCQTSGSGSPNFWGQGTLVTVSSGGGGSGGGSEVQLVESGGGV
VQPGGSLRLSCAASGFTFRSFGMSWVRQAPGKGPEWVSSISGSGSDTLYADSVKGRFTISRDNSKNTLY
LQMNSLRPEDTALYYCTIGGSLSRSSQGTLVTVSSGGGGSGGGSEVQLVESGGGVVQPGGSLRLSCAAS
GRTLSSYAMGWFRQAPGKEREFVARISQGGTAIYYADSVKGRFTISRDNSKNTVYLQMNSLRPEDTALY
YCAKDPSPYYRGSAYLLSGSYDSWGQGTLVKVSSA

Quality of the compound B protein was evaluated by—amongst other techniques—analytical IEX-HPLC (conditions as set forth in Table C, IEX-HPLC protocol II).

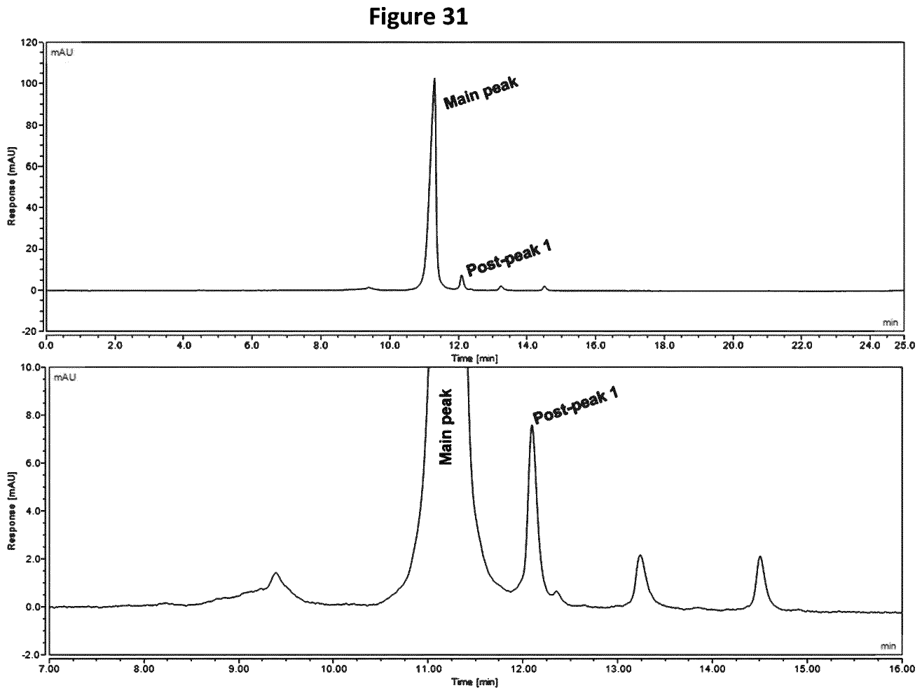

For the purified compound B protein some distinct side peaks were observed in the IEX-HPLC profile (FIG. 31). A 2D-LC multiple heart cutting analysis in line with the mass spectrometer (MS) was performed to identify the variants. By 2D-LC-MS, the top fraction of every peak observed in the IEX-HPLC (1D) profile was collected separately and—after a desalting step (2D)—analyzed on a Q-TOF mass spectrometer resulting in determination of the molecular masses of the protein represented by the IEX peaks. The 2D-LC-MS analysis showed that post-peak 1 has the same molecular mass as the product (main peak), concluding that post-peak 1 is an “intact mass variant” with an altered surface charge distribution compared to the product and so potentially a compact form (data not shown).

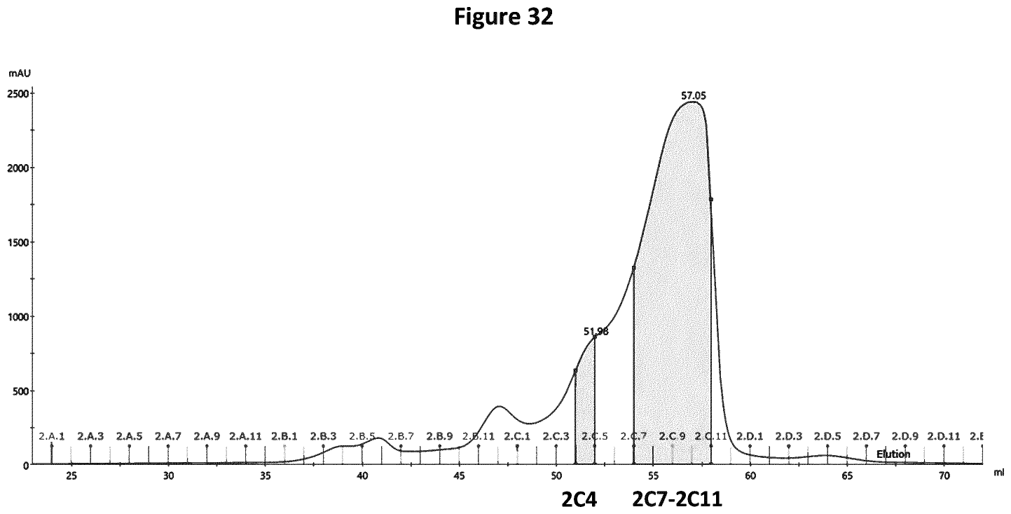

Additionally, during the polish process step of compound B, several CEX (cation exchange chromatography) resins showed a chromatographic profile (i.e., main peak with a pre-peak “shoulder”) similar to those previously observed for compound A on CEX (see e.g., Examples 1 and 2). Therefore, material generated during the polish process step of compound B was subsequently analysed by IEX-HPLC. A gradient using a CEX resin had been performed during polish process, the run conditions are presented in Table 14 and the chromatogram is presented in FIG. 32.

TABLE 14
Conditions for gradient on CEX resin during
polish process step of compound B.
Buffers

| | |
|---|---|
| Equilibration | 50 mM Histidine pH 6.0 |
| Elution | 50 mM Histidine pH 6.0 + 300 mM NaCl |
| CIP | 1M NaOH |
| Storage | 10 mM NaOH |

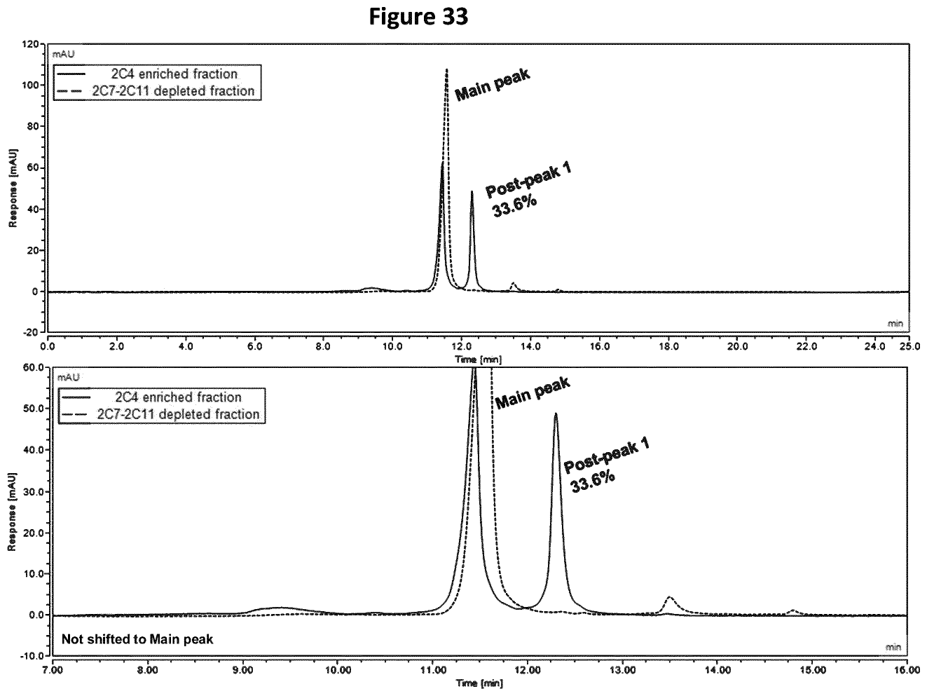

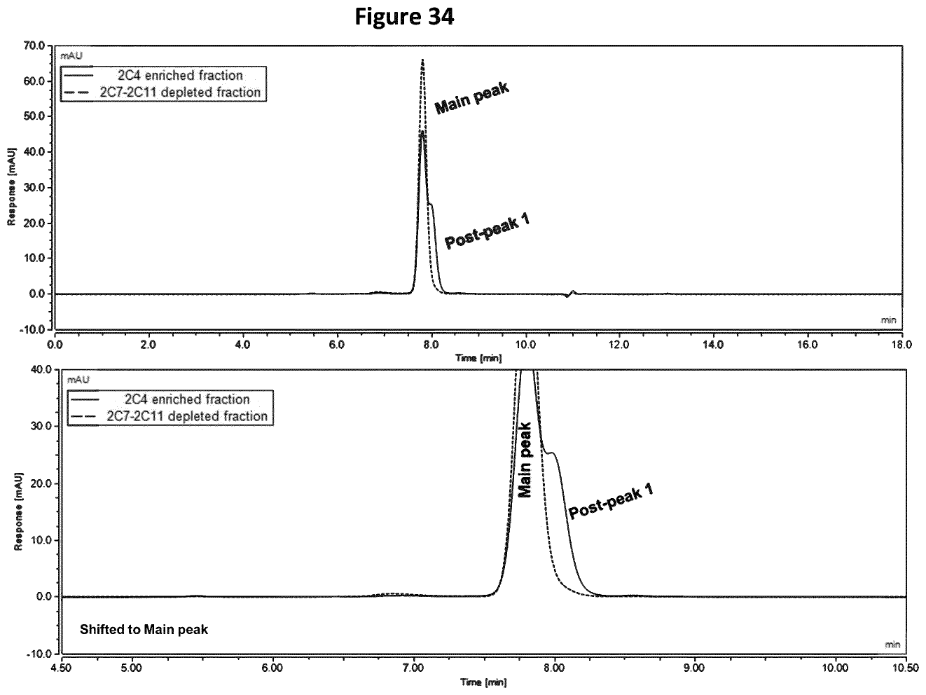

Fraction 2C4 and pool of fractions 2C7-2C11 (FIG. 32) were submitted for IEX-HPLC analysis and SE-HPLC analysis (conditions as set forth in Table C). Results are presented in FIG. 33 and FIG. 34, respectively.

In IEX-HPLC analysis (FIG. 33), fraction 2C4 contained 33.6% of IEX-HPLC post-peak 1, while this variant was <1.0% present in the pool of fractions 2C7-2C11. SE-HPLC results showed a chromatographic profile similar to those observed for compound A with fraction 2C4 displaying a post peak shoulder compared to fractions 2C7-2C11. Together these results implied that IEX-HPLC post-peak 1 could be a “compact” variant that could potentially have an impact on potency as observed for compound A. Therefore, fraction 2C4 and the pool of fractions 2C7-2C11 were submitted for potency analysis.

The potencies of compound B towards TNFα, IL-23 and HSA were determined as described in item 5.4.5:
Cell based reporter assay for the potency testing of the TNF-alpha binding moiety;
Cell based reporter assay for the potency testing of the IL-23 binding moiety;
ELISA based albumin binding assay for potency testing of the albumin binding moiety.

Results of the potency analyses are presented in Table 15.

TABLE 15
Potency results for compound B compact variant-enriched
and -depleted fractions obtained in CEX.

| Sample | HSA | IL-23 | TNFα |
|---|---|---|---|
| Enriched fraction (2C4) | 0.743 | 0.830 | 0.543 |
| depleted fraction (2C7-2C11) | 1.098 | 0.950 | 1.155 |

A significant drop was observed at least for potency towards TNFα for the enriched fraction 2C4 containing 33.6% IEX-HPLC post-peak 1 compared to the pool fraction 2C7-2C11. It was concluded that on top of affecting compound B hydrodynamic volume and charge, the conformational change also impacts at least the binding to TNFα.

Means of removing/converting the compact variant were therefore investigated.

### 6.8 Example 8: Determining Conditions that Influence the Conformation of Compound B

### Low pH Treatment of Compound B

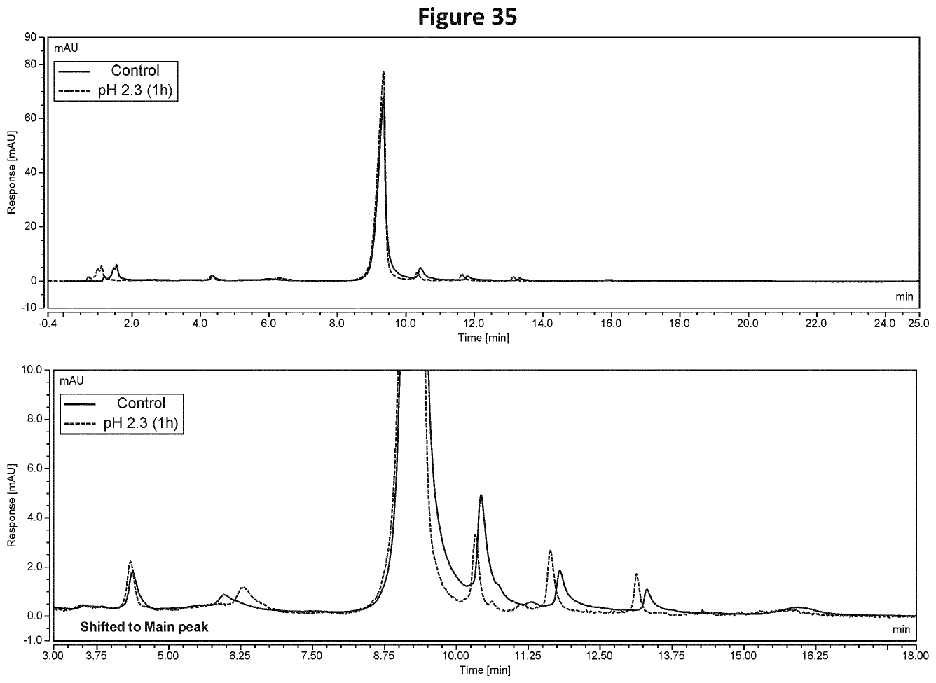

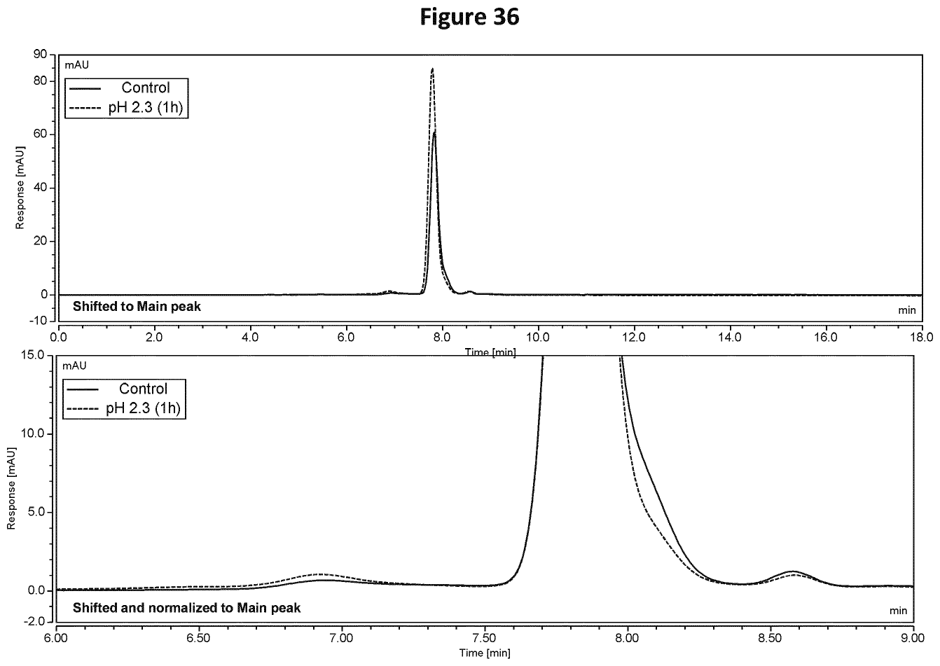

Based on the observations made for compound A, a low pH incubation on capture eluate material of compound B was tested. The pH of the capture eluate was decreased to pH 2.1, pH 2.3 or pH 2.5 with 1M HCl. After a 1 h incubation at low pH, samples were adjusted to pH 5.5 with 1M sodium acetate. The product quality of the different low pH treated samples was compared to the capture eluate directly adjusted to pH 5.5 with 1M sodium acetate (control) and analyzed by IEX-HPLC (Table 16 and FIG. 35) and SE-HPLC (FIG. 36) (conditions as set forth in Example 7 as well as in Table C; IEX-HPLC protocol II).

TABLE 16
IEX-HPLC analysis of the low pH treatment impact.

| | Capture eluate | Capture eluate | Capture eluate | Capture eluate |
|---|---|---|---|---|
| | directly adjusted | 1 h at pH 2.1 and | 1 h at pH 2.3 and | 1 h at pH 2.5 and |
| | to pH 5.5 | adjusted to pH | adjusted to pH | adjusted to pH |
| | (control) | 5.5 | 5.5 | 5.5 |
| IEX-HPLC Main | 84.9 | 86.8 | 87.7 | 87.3 |
| peak (%) | | | | |
| IEX-HPLC post- | 6.8 | 5.1 | 3.2 | 3.4 |
| peak 1 | | | | |

The results in IEX-HPLC analysis show that the low pH treatment leads to an increase in product (% main peak purity) as well as a decrease in compact variant (% IEX-HPLC post-peak 1). Additionally, and similarly to compound A, the main peak observed on SE-HPLC “sharpens” after low pH treatment implying the presence of a compact variant in the capture eluate directly adjusted to pH 5.5. Altogether these results demonstrate the presence of a compact variant that can be converted to product (main peak on IEX-HPLC and/or SE-HPLC) and therefore active product as observed for compound A.

Based on the observations made for compound A and in order to assess if the IEX-HPLC post-peak 1 can be converted, conditions based on chaotropic agents, heat or low pH were tested on compound B. The samples were then analysed by RP-UHPLC, SE-HPLC and IEX-HPLC. Only results with changes relevant to IEX-HPLC post-peak 1 are presented here.

### Low pH Treatment

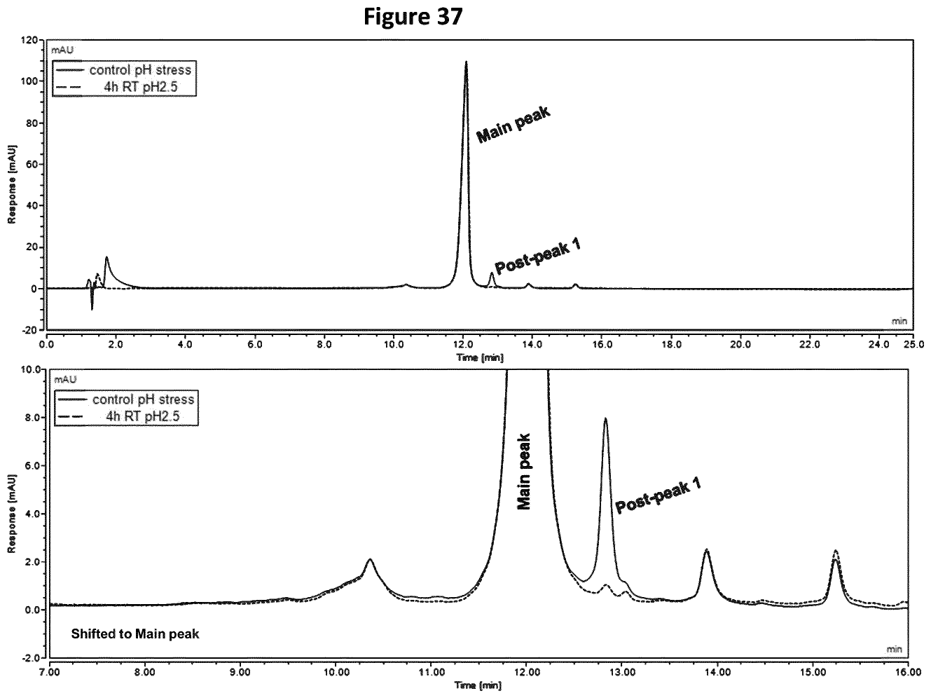

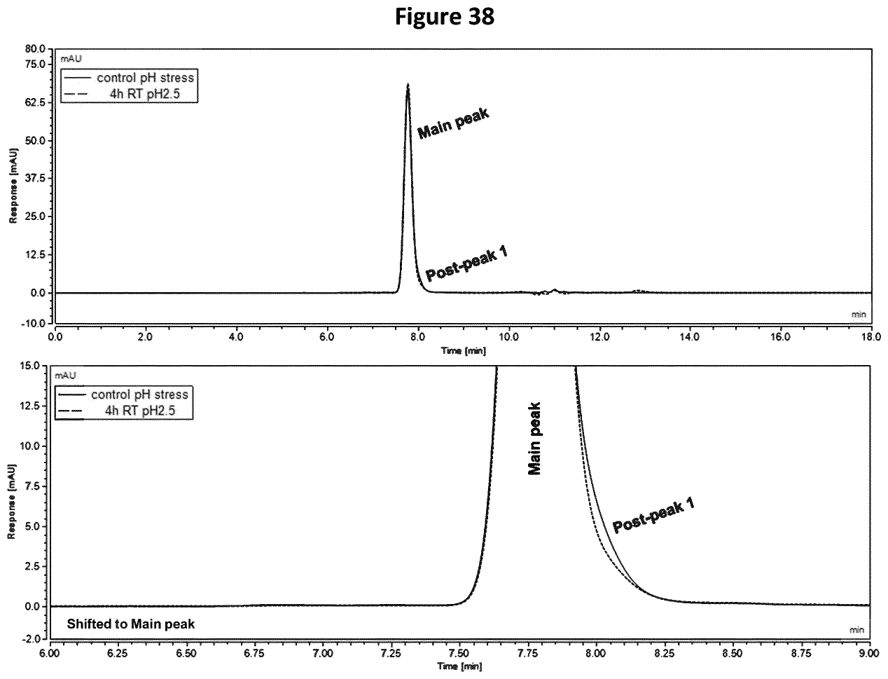

For the low pH treatment, compound B was treated with 100 mM final glycine pH 2.5, pH 3.0 or pH 3.5 or with formulation buffer pH 6.5 (control). After a 4 h incubation at RT, samples were analysed or neutralized with 0.1M NaOH and then analysed. The results of IEX-HPLC and SE-HPLC of the non-neutralized samples are shown in FIGS. 37 and 38, respectively; all results are summarized in Table 17.

TABLE 17
IEX-HPLC and SE-HPLC analysis of the low pH treatment impact.

| | IEX-HPLC | IEX-HPLC | SE-HPLC |
|---|---|---|---|
| | Main peak (%) | Post-peak 1 | HMW species (%) |
| No low pH | 86.8 | 5.2 | 0.4 |
| treatment | | | |
| (control) | | | |
| 4 h at RT pH 2.5 | 90.5 | 1.5 | 0.2 |
| without | | | |
| neutralization | | | |
| 4 h at RT pH 2.5 | 93.3 | 0.8 | 0.3 |
| with | | | |
| neutralization | | | |
| 4 h at RT pH 3.0 | 88.8 | 3.8 | 0.8 |
| without | | | |
| neutralization | | | |
| 4 h at RT pH 3.0 | 87.1 | 4.8 | 0.5 |
| with | | | |
| neutralization | | | |
| 4 h at RT pH 3.5 | 88.6 | 4.8 | 0.7 |
| without | | | |
| neutralization | | | |
| 4 h at RT pH 3.5 | 88.8 | 4.5 | 0.3 |
| with | | | |
| neutralization | | | |

When the sample was treated with 100 mM glycine final pH 2.5 and incubated for 4 h at RT, with or without neutralization, the IEX-HPLC % main peak of compound B increased while the % IEX-HPLC post-peak 1 decreased compared to the control (Table 17 and FIG. 37), implying for IEX-HPLC post-peak 1 to be a conformational variant. IEX-HPLC post-peak 1 can be converted to main peak and therefore active product. Additionally, no significant changes in IEX-HPLC results compared to the control were observed when the sample was treated with 100 mM glycine final pH 3.5 and incubated for 4 h at RT, with or without neutralization and limited decrease for IEX-HPLC post-peak 1 could be observed after pH 3.0 treatment. With regard to SE-HPLC results (Table 17 and FIG. 38), no increase in HMW species was observed indicating that the IEX-HPLC post-peak 1 was not converted into HMW species (e.g., soluble aggregates). Moreover, SE-HPLC results revealed that the pH 2.5 treatment affects the shape of the main peak. The main peak “sharpens” after pH 2.5 treatment which correlates with IEX-HPLC results and results generated on compound A.

Treatment with Chaotropic Agents

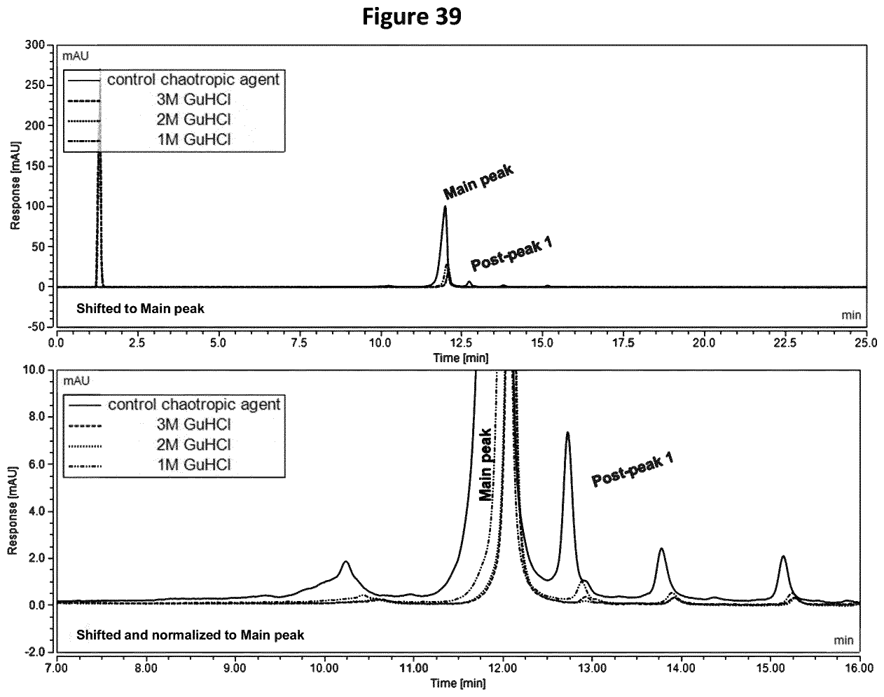

For the treatment with chaotropic agents, compound B was treated with either 3M final guanidine hydrochloride, 2M final guanidine hydrochloride, 1M final guanidine hydrochloride, or Milli Q (control) and was subsequently incubated for 0.5 hours at RT. The results of IEX-HPLC are shown in FIG. 39.

The presence of GuHCl in the sample interfered with the IEX-HPLC method conditions, resulting in a decreased UV signal of the treated samples compared to the control. The integrated data were due to the low signal unreliable (and therefore not shown), but an overlay of the chromatograms indicates that addition of GuHCl could decrease the compact variant peak (IEX-HPLC post-peak 1). These results are in line with the results obtained for compound A.

### Heat Stress Treatment

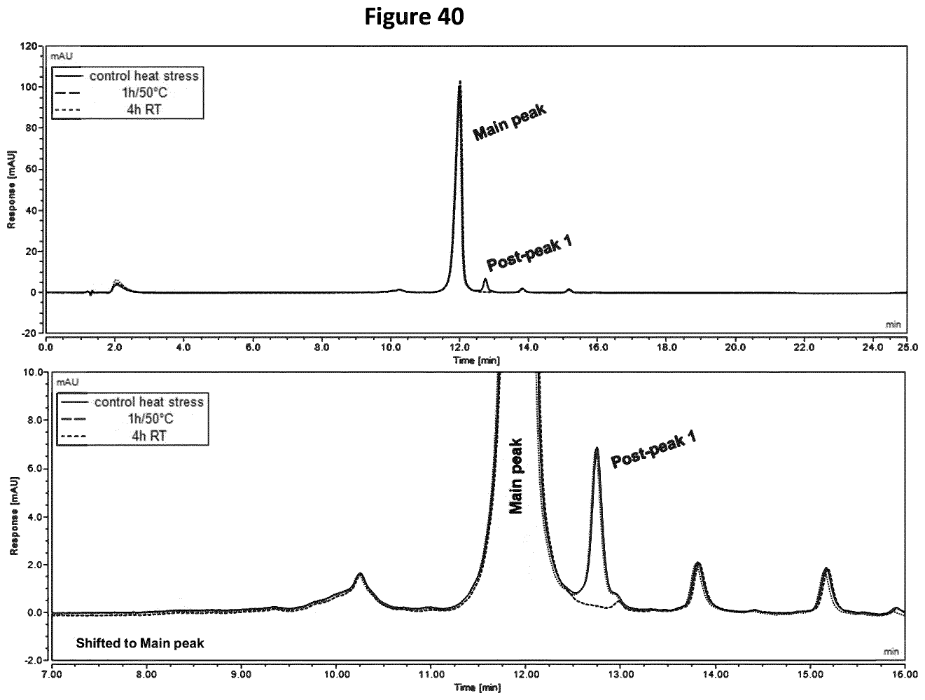

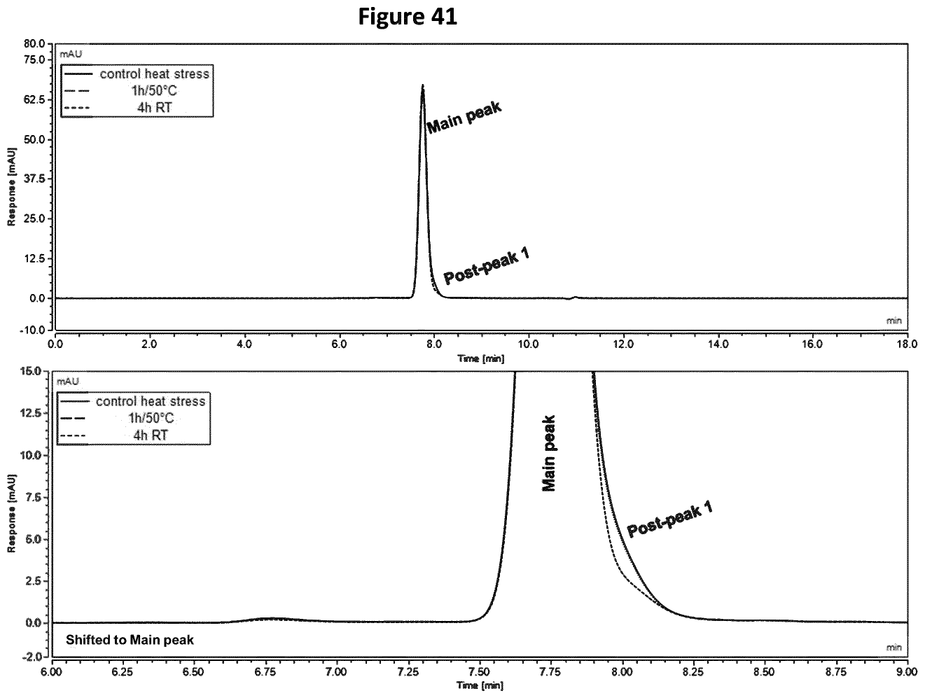

For the treatment with heat, compound B was incubated 1 h at 50° C., 4 h at 50° C., 1 h at 60° C., 4 h at 60° C. (subsequently re-equilibrated to RT), 4 h at RT, or not incubated (control). The results of IEX-HPLC and SE-HPLC are shown in FIGS. 40 and 41 (for 1 h at 50° C.), respectively, and summarized in Table 18.

TABLE 18
Results IEX-HPLC and SE-HPLC analysis of the heat treatment
impact on conversion of the conformational variant.

| | IEX-HPLC | IEX-HPLC | SE-HPLC |
|---|---|---|---|
| | Main peak | Post-peak 1 | HMW species |
| | (%) | (%) | (%) |
| No incubation (control) | 87.9 | 4.8 | 1.0 |
| 1 h at 50° C. | 91.2 | 1.3 | 0.6 |
| 4 h at 50° C. | 92.3 | 1.0 | 0.6 |
| 1 h at 60° C. | 92.7 | 0.9 | 0.6 |
| 4 h at 60° C. | 92.4 | 1.1 | 0.7 |
| 4 h at RT | 88.4 | 4.8 | 0.9 |

When treated with heat for 1 h at 50° C., 4 h at 50° C., 1 h at 60° C., or 4 h at 60° C., the IEX-HPLC % main peak of compound B increased while the % IEX-HPLC post-peak 1 decreased compared to the control, implying for IEX-HPLC post-peak 1 to be a conformational variant (Table 18 and FIG. 40). IEX-HPLC post-peak 1 could potentially be converted to main peak and therefore active product. Additionally, no significant changes compared to the control were observed when incubated for 4 h at RT. When these samples were analyzed by SE-HPLC, no increase in HMW species was observed, indicating that the IEX-HPLC post-peak 1 was not converted into HMW species (e.g. soluble aggregates). Moreover, SE-HPLC results (Table 18 and FIG. 41) revealed that the heat treatment affects the shape of the main peak. The main peak “sharpens” once heat treated which correlates with IEX-HPLC results and results generated on compound A.

### Summary

Altogether, these results confirmed that IEX-HPLC post-peak 1 was a conformational variant of compound B (herein referred to as the less potent “compact variant”) that could be converted to the more potent intact form of the main peak (herein referred to as “intact product”) in IEX-HPLC and SE-HPLC by a low pH treatment at pH 2.5, GuHCl treatment and/or heat treatment.

### 6.9 Example 9: Optimization of Low pH Treatment for Compound B

Based on the treatments for compound A and the compound B results described in Example 8 above, the low pH treatment as a mean of converting a compact variant was optimized for compound B.

After expression of compound B in P. pastoris and harvest, a capture chromatography using Amsphere A3 resin was used to isolate compound B from other impurities.

The column was first equilibrated with PBS buffer pH 7.5 and loaded with clarified cell-free harvest material containing the compound of interest. Compound B binds to the Amsphere A3 resin and impurities flow through the column. Subsequently, the loaded resin was washed with the same PBS buffer as the equilibration step, followed by tris buffer to wash. The tris buffer contained 100 mM tris, and 1M NaCl at pH 8.5. The resin was further washed with a second 100 mM Tris buffer at pH 5.5. Compound B was eluted from the column with a low pH glycine buffer. The low pH glycine elution buffer contained 100 mM Glycine at pH 3. Finally, the resin was cleaned with 100 mM NaOH before storage in the same PBS buffer as equilibration. All buffers are were run at 183 cm/h.

After capture chromatography, the pH of the product eluting from the chromatography column was pH 3.8. Then, a low pH incubation step was applied to compound B.

### Low pH Incubation Time

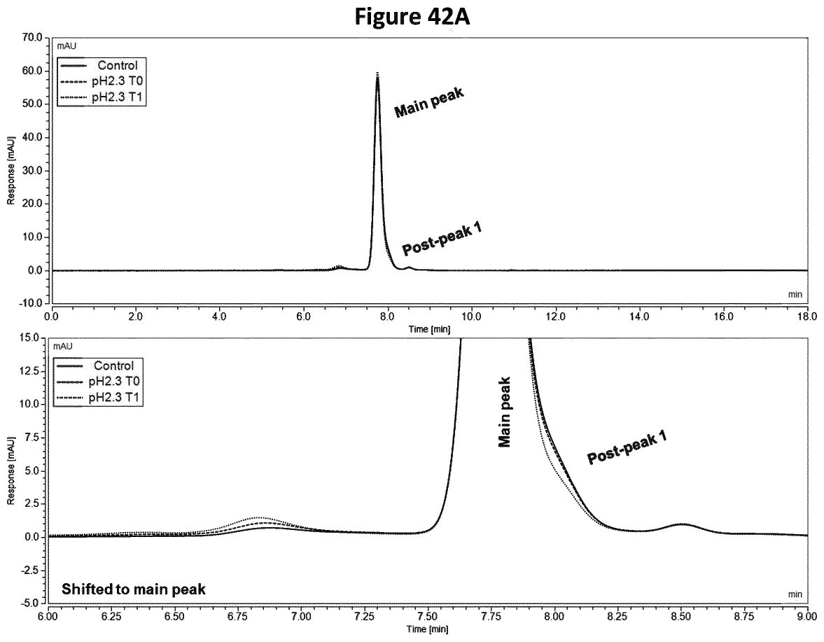

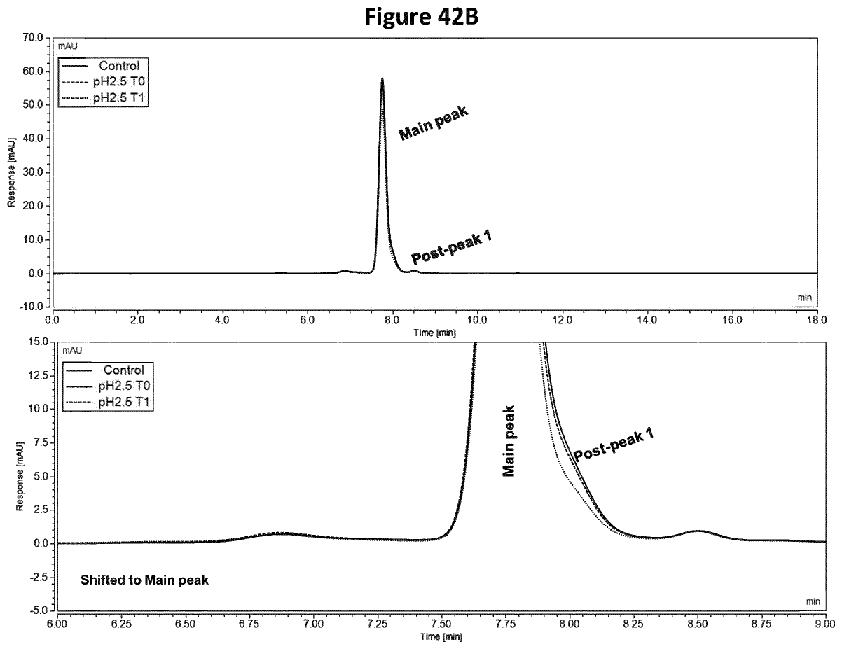

(1) Initial experiment: First, the impact of the low pH treatment at pH 2.3 and pH 2.5 (see Example 1) was confirmed in a subsequent experiment and the length of the incubation at low pH was further assessed. The pH of the capture eluate was decreased to pH 2.3 or pH 2.5 with 1M HCl and was directly adjusted to pH 5.5 with 1M sodium acetate (T0), incubated for 1 h at low pH and then adjusted with 1M sodium acetate (T1), incubated for 2 h at low pH and then adjusted with 1M sodium acetate (T2), or incubated for 4 h at low pH and then adjusted with 1M sodium acetate (T4). The product quality of the different low pH treated samples was compared to the capture eluate directly adjusted to pH 5.5 with 1M sodium acetate (control) and analysed by IEX-HPLC, SE-HPLC and CGE (conditions as set forth in Table C; IEX-HPLC protocol II). The results of SE-HPLC are shown in FIGS. 42A and 42B and summarized in Table 19.

TABLE 19
Results of IEX-HPLC, SE-HPLC and CGE analysis of the low pH
treatment impact on conversion of the conformational variant.

| | IEX-HPLC | IEX-HPLC | SE-HPLC | CGE |
|---|---|---|---|---|
| | Main peak | Post-peak 1 | HMW species | Main peak |
| | (%) | (%) | (%) | (%) |
| No low pH | 81.9 | 4.4 | 3.3 | 90 |
| treatment | | | | |
| (control) | | | | |
| pH 2.3 T0 | 81.5 | 4.5 | 4.8 | 90 |
| pH 2.3 T1 | 85.7 | 1.2 | 5.7 | 90 |
| pH 2.3 T2 | 84.1 | 1.5 | 5.1 | 91 |
| pH 2.3 T4 | 87.1 | 0.6 | 4.9 | 90 |
| pH 2.5 T0 | 81.7 | 4.3 | 3.8 | 90 |
| pH 2.5 T1 | 83.3 | 2.4 | 3.9 | 90 |
| pH 2.5 T2 | 85.1 | 1.4 | 3.5 | 90 |
| pH 2.5 T4 | 87.4 | 0.7 | 3.7 | 91 |

With regard to IEX-HPLC results (Table 19), no differences were observed between the control, pH 2.3 T0 and pH 2.5 T0. A significant increase in % main peak purity as well as a decrease in % IEX-HPLC post-peak 1 (compact variant) was observed for a 1 h incubation, 2 h incubation and 4 h incubation at low pH. Furthermore, the reduction in IEX-HPLC post-peak 1 was the most efficient at the longest incubation time. With regard to SE-HPLC results (Table 19, FIGS. 42A and 42B), decreasing the pH of the capture eluate to pH 2.3 or pH 2.5 led to a slight increase in HMW species (pre-peaks) but also mainly to a narrowing of the main peak as observed previously. CGE profiles (Table 19) did not show significant differences between the different samples for main peak purity, confirming the initial 2D-LC results (Example 7) that the compact variant does not have a different molecular weight than the intact product. Altogether, these results confirmed that IEX-HPLC post-peak 1 was a compact variant that could be converted to main peak in IEX-HPLC and SE-HPLC by a low pH 2.3 and pH 2.5 treatment for 1 h, 2 h and 4 h.

(2) Additional experiment: the low pH treatment of the initial experiment was then broadened. The pH of the capture material of compound B was decreased to pH 2.7, pH 2.9, pH 3.1, pH 3.3, pH 3.5, and pH 3.9 with 1M HCl. After a 2 h and 4 h of incubation at low pH, samples were adjusted to pH 5.5 with 1M sodium acetate.

The T0 was generated by decreasing the pH of the capture eluate of compound B to the target low pH (i.e., pH 2.7 to 3.9 as indicated above) with 1M HCl and directly adjusting to pH 5.5 with 1M sodium acetate (T0).

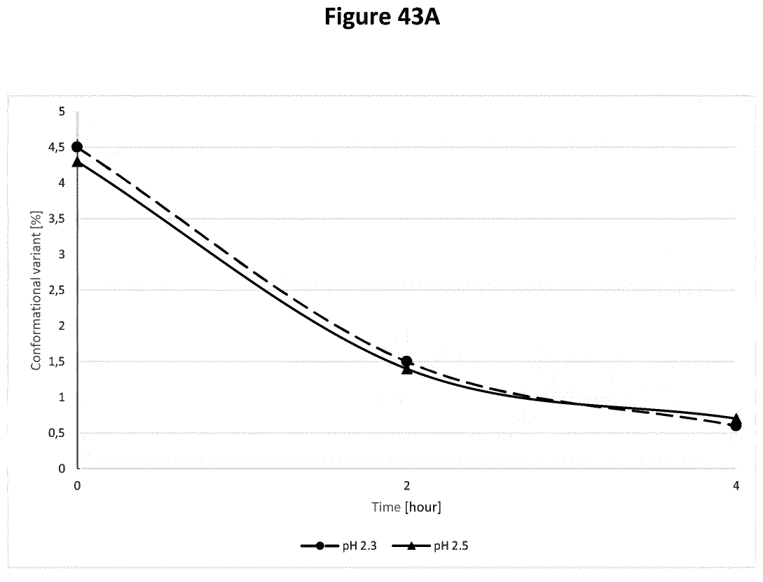

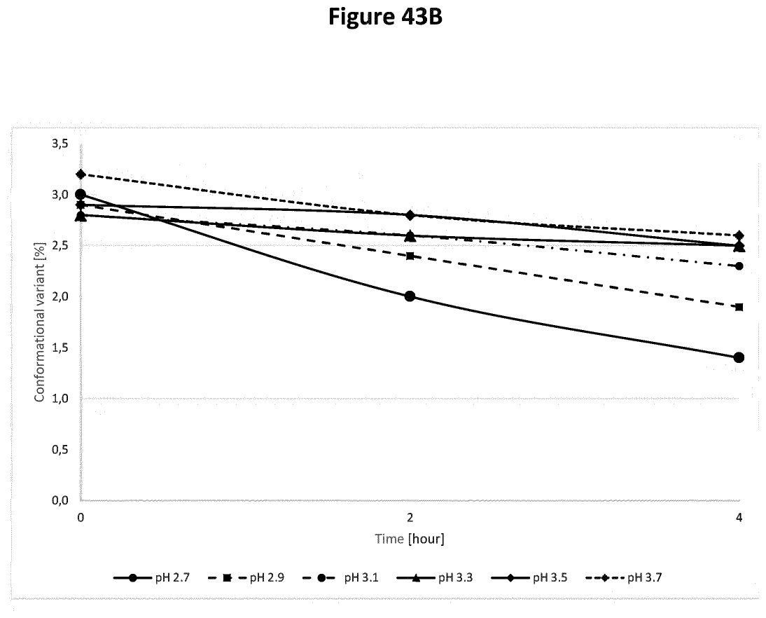

The influence of the low pH treatment on the product quality in function of time was analysed by IEX-HPLC. See Table 20 and FIGS. 43A and B.

TABLE 20
Results of IEX-HPLC analysis of the low pH treatment impact
on conversion of the conformational variant. Also included
are the T0, T2, and T4 results observed for pH treatment at
pH 2.3 and pH 2.5 of the initial experiment of Example 9 above.

| pH | Time point [hour] | IEX-HPLC Post-peak 1 (%) |
|---|---|---|
| 2.3 | 0 | 4.5 |
| 2.3 | 2 | 1.5 |
| 2.3 | 4 | 0.6 |
| 2.5 | 0 | 4.3 |
| 2.5 | 2 | 1.4 |
| 2.5 | 4 | 0.7 |
| 2.7 | 0 | 3.0 |
| 2.7 | 2 | 2.0 |
| 2.7 | 4 | 1.4 |
| 2.9 | 0 | 2.9 |
| 2.9 | 2 | 2.4 |
| 2.9 | 4 | 1.9 |
| 3.1 | 0 | 2.8 |
| 3.1 | 2 | 2.6 |
| 3.1 | 4 | 2.3 |
| 3.3 | 0 | 2.8 |
| 3.3 | 2 | 2.6 |
| 3.3 | 4 | 2.5 |
| 3.5 | 0 | 2.9 |
| 3.5 | 2 | 2.8 |
| 3.5 | 4 | 2.5 |
| 3.7 | 0 | 3.2 |
| 3.7 | 2 | 2.8 |
| 3.7 | 4 | 2.6 |

The IEX-HPLC results show the positive influence of a low pH treatment on the presence of conformational variant in the sample. The level of conformational variant in the T0 sample was similar in all samples tested. In the initial set of experiments, i.e, pH 2.3 and 2.5, the level was at about 4.5%. In the additional experiment, the level of conformational variant in the control samples at T0 (pH 2.7, 2.9, 3.1, 3.3, 3.5 and pH 3.7) was about 3%.

After 2 h of incubation at low pH, the level of conformational variant decreased in all pHs tested. The positive effect of low pH on the conformational variant increased with lower pHs, i.e., below pH 3.0.

After 4 h of incubation at low pH, the level of conformational variant further decreased for all pH tested. The best reduction was obtained at pH 2.3 up to pH 2.9.

All results obtained in this example show the positive impact of low pH on the conformational variant, especially at pH 3 or less.

### Additional Low pH Treatments

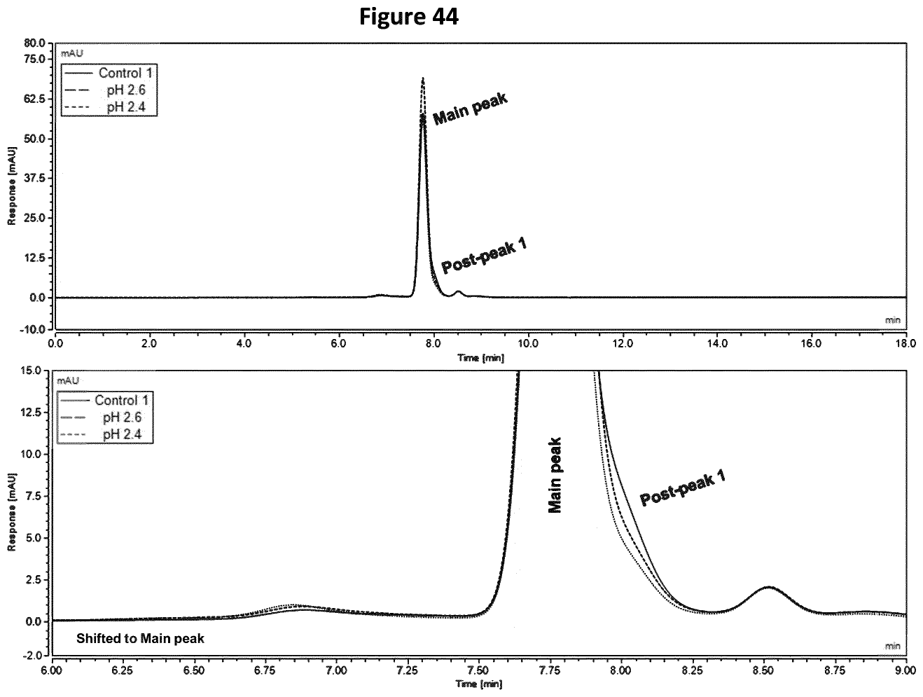

Then, in order to investigate the broadness of the working range of the low pH treatment, a 2 h low pH incubation at pH 2.4 and pH 2.6 was investigated. The pH of the capture eluate was decreased to pH 2.4 or pH 2.6 with 1M HCl and the samples were incubated at RT for 2 h. The samples were then adjusted to pH 5.5 with 1M sodium acetate. The product quality of the different low pH treated samples was compared to the capture eluate directly adjusted to pH 5.5 with 1M sodium acetate (control) and analyzed by IEX-HPLC, SE-HPLC and CGE. The results are shown in FIG. 44 and summarized in Table 21.

TABLE 21
Results of IEX-HPLC, SE-HPLC and CGE analysis of the low pH
treatment impact on conversion of the conformational variant.

| | IEX-HPLC | IEX-HPLC | SE-HPLC | CGE |
|---|---|---|---|---|
| | Main peak | Post-peak 1 | HMW species | Main peak |
| | (%) | (%) | (%) | (%) |
| No low pH | 78.6 | 5.5 | 3.0 | 87 |
| treatment | | | | |
| (control) | | | | |
| pH 2.4 2 h | 84.5 | 1.4 | 4.3 | 87 |
| pH 2.6 2 h | 84.3 | 1.7 | 3.7 | 87 |

The IEX-HPLC results (Table 21) show a significant increase in % main peak purity as well as a decrease in % IEX-HPLC post-peak 1 (compact variant) after 2 h incubation at pH 2.4 and pH 2.6. The SE-HPLC results (Table 21 and FIG. 44) show that decreasing the pH of the capture eluate to pH 2.4 or pH 2.6 led to a slight increase in HMW species, but also to a narrowing of the main peak as observed previously. The CGE profiles (Table 21) did not show significant differences between the control and the low pH treated samples, confirming the initial 2D-LC results (Example 7) that the compact variant does not have a different molecular weight than the intact product. Altogether, these results confirmed that IEX-HPLC post-peak 1 was a conformational variant that could be converted to main peak intact form in IEX-HPLC by a pH 2.4 and 2.6 treatment for 2 h.

### Low pH Adjustment Procedure

Finally, the procedure to adapt the pH was investigated in order to consider the impact on the next purification step of the process. Indeed, by increasing the pH with 1M sodium acetate after the low pH treatment to reach pH 5.5, the conductivity of the sample rose significantly. The sample had then to be highly diluted with water to a conductivity adequate to the next chromatography step (≤6.0 mS/cm). This significantly increased the load volume and consequently the process time.

The different approaches for adjusting the pH after low pH treatment were performed in two independent experiments (Table 22). In experiment 1, the capture eluate was either directly adjusted to pH 5.5 and conductivity≤6.0 mS/cm with 1M sodium acetate pH 9 (control 1) or the capture eluate was first adjusted to pH 2.4 with 1M HCl for 2 h, then adjusted to pH 5.5 with 1M sodium acetate and diluted with MilliQ water to reach a conductivity (≤6.0 mS/cm).

In experiment 2, the capture eluate was either directly adjusted to pH 5.5 and conductivity≤6.0 mS/cm with 1M sodium acetate pH 9 (control 2) or the capture eluate was first adjusted to pH 2.6 with 1M HCl for 2 h, then adjusted to pH 5.5 and conductivity 6.0 mS/cm by (i) adding a given volume of 1M sodium acetate pH 5.5 to reach z50 mM sodium acetate, (ii) adjusting to pH 5.5 with 0.1M NaOH and (iii) adjusting to conductivity 6.0 mS/cm with water if necessary.

TABLE 22
Impact of different pH adjustment approaches for the low pH treatment.

| | Experiment 1 | | | |
|---|---|---|---|---|
| | | Capture eluate | Experiment 2 | |
| | Capture eluate | 2 h at pH 2.4 | Capture eluate | Capture eluate |
| | directly adjusted | and adjusted | directly adjusted | 2 h at pH 2.6 |
| | to pH 5.5 with | to pH 5.5 with | to pH 5.5 with | and adjusted |
| | 1M sodium acetate | 1M sodium acetate | 1M sodium acetate | to pH 5.5 with |
| | pH 9 (control 1) | pH 9 and MilliQ. | pH 9 (control 2) | new approach |
| IEX-HPLC Main peak (%) | 78.6 | NA^{a} | 79.7 | 84.2 |
| IEX-HPLC Post-peak 1 (%) | 5.5 | NA^{a} | 5.6 | 1.7 |
| SE-HPLC HMW species (%) | 3.0 | NA^{a} | 3.0 | 2.8 |
| CGE Main peak (%) | 87 | NA^{a} | 87 | 87 |
| Conductivity final (mS/cm) | 4.92 | 4.97 | 5.4 | 6.0 |
| Dilution factor (volume adjusted | 1.06 | 10.08 | 1.08 | 1.74 |
| eluate pH 5.5/volume capture eluate) | | | | |

^{a}NA: not applicable (not tested); see Table 21 for data obtained under comparative conditions.

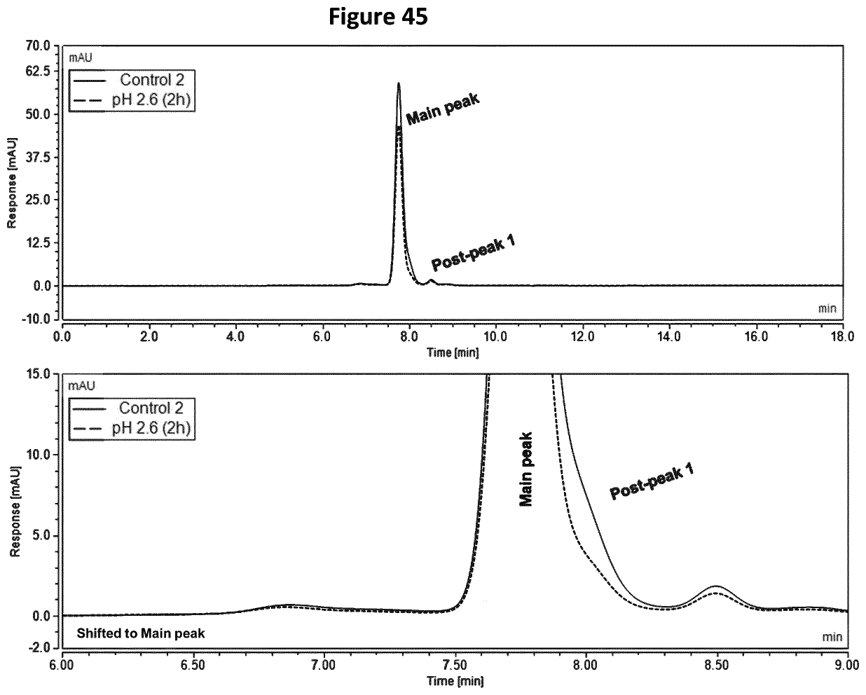

Independently of the approach for increasing the pH to pH 5.5 after the low pH treatment (Table 21 and Table 22), a similar decrease in % IEX-HPLC post-peak 1 was observed on IEX-HPLC. Surprisingly, compared to previous compound B results, there was no increase in HMW species observed in SE-HPLC with the new pH adjustment approach (mix 1M sodium acetate pH 5.5 and 0.1 M NaOH) (Table 22 and FIG. 45). Moreover, the narrowing of the SE-HPLC main peak was still observed after low pH treatment at pH 2.6 and the new pH adjustment approach (FIG. 45). Finally, the dilution factor (volume adjusted eluate pH 5.5/volume capture eluate) was significantly lower with the new pH adjustment approach (Table 22) therefore improving the overall process time by decreasing the volume to be processed on the next purification step.

### 6.10 Example 10: Impact of Low pH Treatment on Compound B

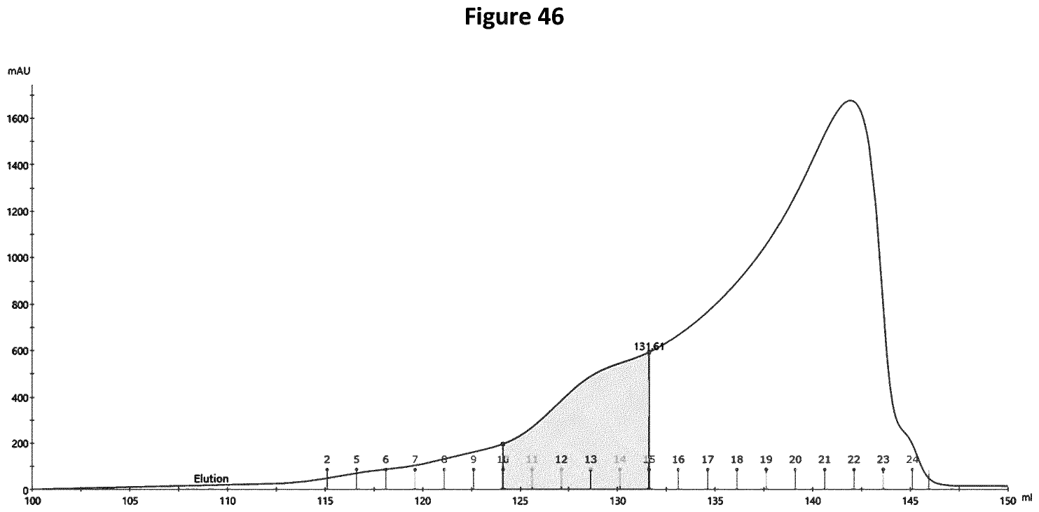

As the initial characterization showed a drop in potency for fractions enriched in IEX-HPLC post-peak 1 (i.e., compact variant) and as the low pH treatment converts compound B compact variant into the more active intact product, the impact of the low pH treatment on compound B conformational variant was hereunder investigated in order to assess if the potency was restored. A gradient using a CEX resin was performed, with run conditions as presented in Table 23. The chromatogram is presented in FIG. 46.

TABLE 23
Conditions for gradient on CEX resin for
compound B compact variant enrichment.
Buffers

| | |
|---|---|
| Equilibration | 25 mM sodium acetate pH 5.5 |
| Elution | 25 mM sodium acetate pH 5.5 + 175 mM NaCl |
| CIP | 1M NaOH |
| Storage | 10 mM NaOH |
| Run number | B23/190207/1 |

The CEX chromatogram displayed the expected main peak shoulder containing the compact variant. A pool of fractions 10-14 (FIG. 46) was submitted to IEX-HPLC analysis (conditions as set forth in Table C; IEX-HPLC protocol II) without low pH treatment or after low pH treatment at pH 2.5. A summary of the IEX-HPLC results is presented in Table 24.

TABLE 24
Results of IEX-HPLC of the compact variant enriched fraction obtained
in CEX chromatography with or without low pH treatment.

| | IEX-HPLC Main peak (%) | IEX-HPLC Post-peak 1 (%) |
|---|---|---|
| fractions 10-14 | 63.3 | 19.5 |
| without low pH | | |
| treatment | | |
| fractions 10-14 | 75.0 | 8.0 |
| with low pH | | |
| treatment at | | |
| pH 2.5 | | |

The low pH treatment converted the IEX-HPLC post-peak 1 compact variant to the main peak intact product, as is evidenced by the reduction of IEX-HPLC post-peak 1 from 19.5% to 8.0%. The low pH treated sample was submitted for potency analysis and compared with results generated previously (Table 25). The low pH treatment restored the potency, especially towards TNFα, by converting the compact variant into active product. The low pH treatment is therefore a mean of converting the compound B compact variant into the active intact product.

TABLE 25
Results of potency analysis.

| | IEX-HPLC | | | |
|---|---|---|---|---|
| | Post-peak 1 | | | |
| Sample | (%) | HSA | IL-23 | TNFα |
| Enriched fraction (2C4) | 33.6 | 0.743 | 0.830 | 0.543 |
| Main peak fraction depleted | 0.9 | 1.098 | 0.950 | 1.155 |
| fraction (2C7-2C11) | | | | |
| Fraction 10-14 with low | 8.0 | 1.050 | 0.723 | 0.819 |
| pH treatment at pH 2.5 | | | | |

### 6.11 Example 11: Use of HIC for the Removal of the Less Potent Compact Variant of Compound B

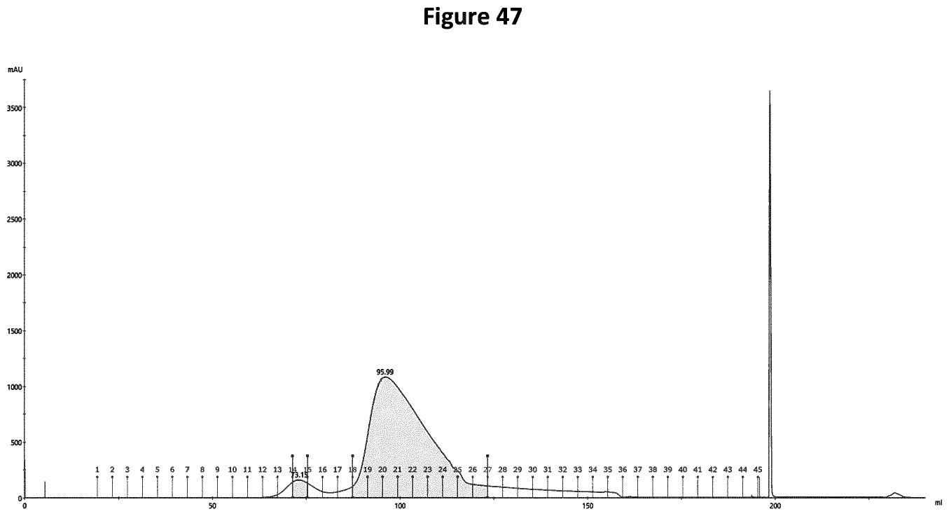

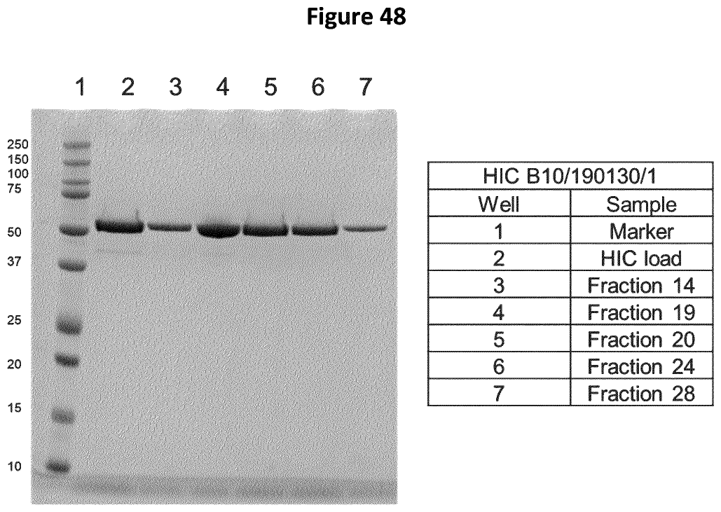

As hydrophobic interaction chromatography (HIC) was successful for removing/enriching compound A compact variant, HIC was also tested for removal of the compact variant of compound B. A gradient using Capto Butyl ImpRes resin (GE Healthcare) was performed with run conditions as presented in Table 26. The chromatogram is presented in FIG. 47. HIC load (polish eluate buffer exchanged in suitable loading condition) and elution fractions 14/19/20/24/28 were analysed by SDS-PAGE (FIG. 48). Fraction 14 and fractions 18 to 26 were analysed by IEX-HPLC (Table 27).

TABLE 26
Conditions for gradient on Capto Butyl ImpRes resin for
the removal of compound B compact variant.
Buffers

| | |
|---|---|
| Loading condition/ | 50 mM Sodium Phosphate pH 6.0 + |
| Equilibration | 0.4M Ammonium sulfate |
| Elution | 50 mM Sodium Phosphate pH 6.0 |
| CIP | 1M NaOH |
| Storage | 10mM NaOH |

TABLE 27
Results of IEX-HPLC (conditions as set forth in Table C;
protocol II) of the different fractions obtained in HIC.

| Fraction | | |
|---|---|---|
| number | IEX-HPLC Main peak (%) | IEX-HPLC post-peak 1 (%) |
| 14 | 47.9 | 52.1 |
| 18 | 68.4 | n.d.^{a} |
| 19 | 96.5 | n.d.^{a} |
| 20 | 98.4 | n.d.^{a} |
| 21 | 96.9 | n.d.^{a} |
| 22 | 96.9 | n.d.^{a} |
| 23 | 96.7 | n.d.^{a} |
| 24 | 95.6 | n.d.^{a} |
| 25 | 94.8 | n.d.^{a} |
| 26 | 93.5 | n.d.^{a} |

^{a}n.d.: not detected

As observed on the chromatographic HIC profile (FIG. 47), the gradient led to 2 separated peaks. The SDS-PAGE analysis (FIG. 48) showed that the main band of the different fractions had a similar molecular weight, as was expected for the compact variant. Interestingly, the IEX-HPLC analysis (Table 27) revealed that only the first peak of the HIC profile (fraction 14) contained the active product and the less active compact variant, with 47.9% of “intact product” and 52.1% of “compact variant” respectively. Moreover, the IEX-HPLC analysis (Table 27) showed that no compact variant was present in the second peak of the HIC profile (fraction 19-26). A conformational variant of compound B could therefore be completely removed and/or enriched for by hydrophobic interaction chromatography.

### 6.12 Example 12: Removal/Reduction of the Less Potent Compact Variant by Increasing the Load Factor on the Capture Column

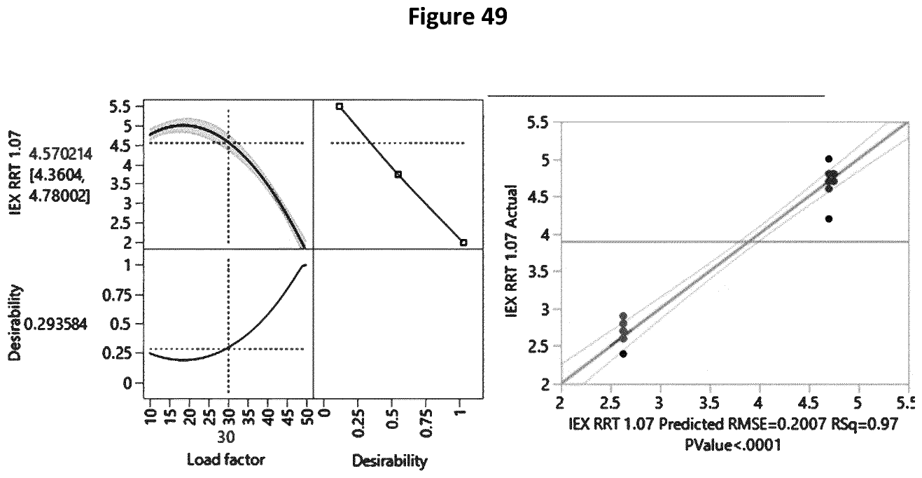

In order to optimize the capture step for compound B, different parameters (i.e. factors) such as load factor (mg product/ml resin), load flow rate (cm/h), pH of elution buffer, load pH and wash buffers of the purification process were assessed using a design of experiment (DOE) approach using Definitive Screening Design (DSD) from JMP (SAS Institute) software. Different outputs (i.e. responses) were measured in order to assess the impact of the factors on the responses. Responses included, but were not limited to, IEX-HPLC analysis in order to assess whether it is possible to reduce/remove the IEX-HPLC post-peak 1 during the capture step. DOE results were analyzed by JMP software following the DSD approach. Interestingly, out of the different factors tested, only the load factor had an impact on the IEX-HPLC post-peak 1 (FIG. 49). Surprisingly, the compact variant IEX-HPLC post-peak 1 could be significantly removed/reduced by increasing the load factor (Table 28). Therefore, increasing the load factor on the capture column with ISVD product could be used as a mean of reducing/removing the undesired less potent compact variant.

TABLE 28
Results of IEX-HPLC (conditions as set forth in Table
C; protocol II) of the capture eluate during DOE.

| | Load | IEX-HPLC | IEX-HPLC |
|---|---|---|---|
| | factor | Main peak | post-peak 1 |
| Run number | (mg/mL) | (%) | (%) |
| DOE Run 1 | 45 | 87.6 | 2.6 |
| DOE Run 2 | 9 | 83.6 | 4.8 |
| DOE Run 3 | 27 | 83.1 | 4.7 |
| DOE Run 4 | 45 | 87.1 | 2.8 |
| DOE Run 5 | 27 | 82.3 | 4.7 |
| DOE Run 6 | 45 | 86.6 | 2.6 |
| DOE Run 7 | 27 | 81.9 | 4.8 |
| DOE Run 8 | 9 | 83.1 | 4.2 |
| DOE Run 9 | 27 | 82.6 | 4.8 |
| DOE Run 10 | 9 | 83.0 | 4.6 |
| DOE Run 11 | 9 | 83.1 | 4.7 |
| DOE Run 12 | 9 | 82.8 | 4.8 |
| DOE Run 13 | 45 | 87.6 | 2.4 |
| DOE Run 14 | 45 | 86.8 | 2.9 |
| DOE Run 15 | 45 | 87.5 | 2.7 |
| DOE Run 16 | 9 | 83.2 | 5.0 |
| DOE Run 17 | 45 | 88.4 | 2.4 |
| DOE Run 18 | 9 | 83.5 | 4.8 |

### 6.13 Example 13: Scale Up of the Low pH Treatment of Compound B (10 L and 100 L)

Based on examples above, the conditions selected for the low pH incubation of compound B were a target pH of 2.5 for 2 h at room temperature. The pH of the capture eluate was lowered using 1M HCl and then adjusted to pH 5.5 and conductivity≤6.0 mS/cm after 2 h by (i) adding a given volume of 1M sodium acetate pH 5.5 to reach≈50 mM sodium acetate, (ii) adjusting to pH 5.5 with 0.1 M NaOH and (iii) adjusting to conductivity 6.0 mS/cm with water if necessary. The production process for compound B was then scaled up to fermentation scales of 10 L and 100 L for further purification. The analytical methods SE-HPLC, IEX-HPLC, CGE were used to analyze the product quality of the capture eluate before low pH treatment (i.e., capture eluate) and of the capture eluate after low pH treatment followed by pH adjustment to 5.5 as described above and filtration (i.e., capture filtrate). 2 cycles of capture step were performed for each scale. The results for the different scales are presented in Table 29.

TABLE 29
Impact of the low pH treatment on the product
quality of compound B during scaling up.

| | IEX-HPLC | IEX-HPLC | SE-HPLC | CGE |
|---|---|---|---|---|
| | Main peak | Post-peak 1 | HMW species | Main peak |
| | (%) | (%) | (%) | (%) |
| 10 L capture | 71.5 | 4.6 | 3.8 | 83.2 |
| eluate cycle 1 | | | | |
| 10 L capture | 76.9 | 1.1 | 2.6 | 83.7 |
| filtrate cycle 1 | | | | |
| 10 L capture | 70.8 | 4.5 | 3.3 | 82.8 |
| eluate cycle 2 | | | | |
| 10 L capture | 76.3 | 1.2 | 2.6 | 83.4 |
| filtrate cycle 2 | | | | |
| 100 L capture | 70.9 | 4.4 | 3.0 | 82.3 |
| eluate cycle 1 | | | | |
| 100 L capture | 74.8 | 1.3 | 2.9 | 82.4 |
| filtrate cycle 1 | | | | |
| 100 L capture | 71.4 | 4.3 | 3.0 | 81.6 |
| eluate cycle 2 | | | | |
| 100 L capture | 73.8 | 1.4 | 2.6 | 82.6 |
| filtrate cycle 2 | | | | |

(SE-HPLC and IEX-HPLC conditions as set forth in Table C; IEX-HPLC protocol II)

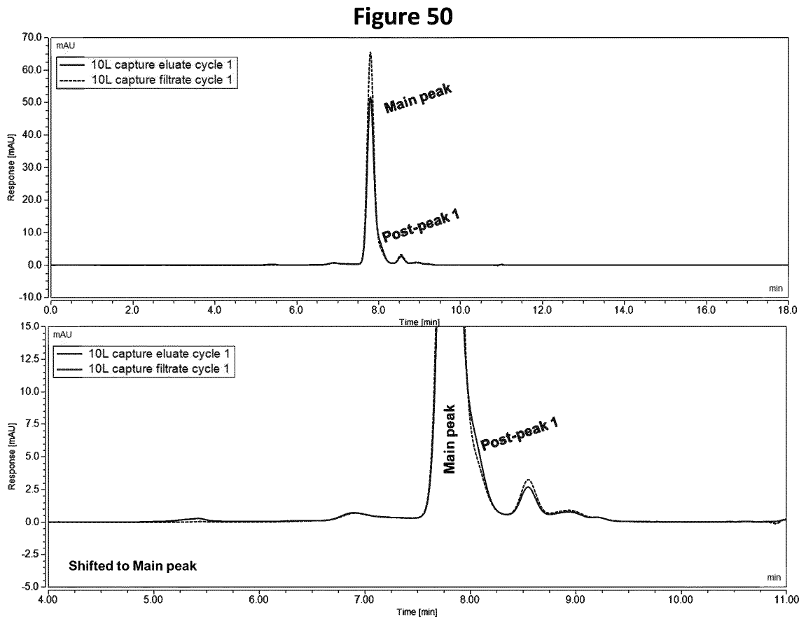

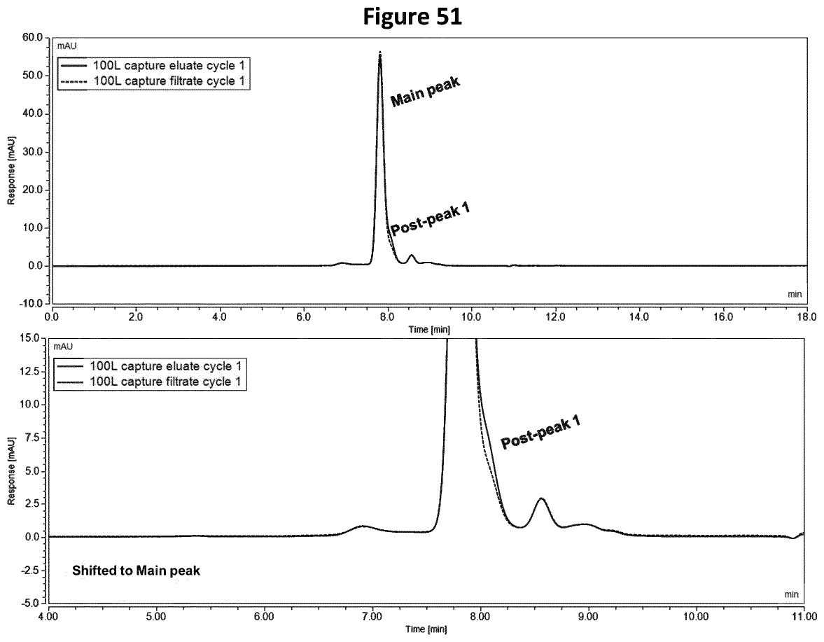

First, independently of the fermentation and purification scales, the low pH treatment and filtration step had no impact on the product quality with regard to % main peak on CGE analysis and the CGE profiles, of which the results were within method variability (Table 29). Surprisingly, a decrease in % HMW species was observed in both scale up when comparing the capture filtrates and the capture eluates that could therefore be due to the low pH treatment and/or the filtration step (Table 29). Additionally, SE-HPLC results (FIG. 50 and FIG. 51) confirmed that the low pH treatment affects the shape of the main peak. The main peak “sharpens” after low pH treatment (e.g. in capture filtrate) which correlates with IEX-HPLC results and results generated for compound A. Finally, as observed before at smaller scale, a significant increase in % main peak purity as well as a decrease in % IEX-HPLC post-peak 1 (compact variant) was observed on IEX-HPLC after low pH treatment when comparing capture filtrates with capture eluates (Table 29).

Altogether, the results showed that the low pH treatment was a scalable process and was efficient at converting a less potent and undesired compact variant of a multivalent ISVD construct in the potent intact product.

### 6.14 Example 14: Identification and Initial Characterization of a Compact Variant of Compound C

In order to confirm that a compact variant also appears for other multivalent ISVD constructs, further investigations were made for compound C.

Compound C (SEQ ID NO: 69) is a multivalent ISVD construct comprising three immunoglobulin single variable domains of heavy-chain llama antibodies that bind to two different targets. The ISVD building blocks are fused head-to-tail (N-terminus to C-terminus) with a G/S linker in the following format: an TNFα-binding ISVD—9GS linker—a human serum albumin-binding ISVD—9GS linker—a TNFα-binding ISVD and have the following sequence:

TABLE 30
Amino acid sequence of compound C.
Compound C (SEQ ID NO: 69)

EVQLVESGGGLVQPGGSLRLSCAASGFTFSDYWMYWVRQAPGKGLEWVSEINTNGLITKYPDSVKGRF
TISRDNAKNTLYLQMNSLRPEDTAVYYCARSPSGFNRGQGTLVTVSSGGGGSGGGSEVQLVESGGGLV
QPGNSLRLSCAASGFTFSSFGMSWVRQAPGKGLEWVSSISGSGSDTLYADSVKGRFTISRDNAKTTLYLQ
MNSLRPEDTAVYYCTIGGSLSRSSQGTLVTVSSGGGGSGGGSEVQLVESGGGLVQPGGSLRLSCAASGF
TFSDYWMYWVRQAPGKGLEWVSEINTNGLITKYPDSVKGRFTISRDNAKNTLYLQMNSLRPEDTA
VYYCARSPSGFNRGQGTLVTVSS

After expression of compound C in P. pastoris and harvesting of the compound by tangential flow filtration, a capture chromatography using Amsphere A3 resin was used to isolate compound C from other impurities.

The column was first equilibrated with PBS buffer pH 7.3 and loaded with clarified cell-free harvest material containing compound C. Compound C binds to the Amsphere A3 resin and impurities flow through the column. Subsequently, the loaded resin was washed with the same PBS buffer as the equilibration step. Compound C was eluted from the column with a low pH glycine buffer. The low pH glycine elution buffer contained 100 mM Glycine at pH 3.0. Finally, the resin was cleaned with 100 mM NaOH before storage in the same PBS buffer as equilibration. All buffers were run at 183 cm/h.

After capture chromatography, pH of the product eluting from the chromatography column was pH 3.5. Compound C was subsequently submitted to low pH incubation. The pH of the capture eluate was decreased to pH 2.5 or pH 3.0 with 1M HCl. After a 2 h and 4 h of incubation at low pH, samples were adjusted to pH 5.5 with 1M sodium acetate pH6.0. The T0 was generated by decreasing compound C to the target low pH (i.e pH 2.5 or 3.0) with 1M HCl and directly adjusted to pH 5.5 with 1M sodium acetate (T0).

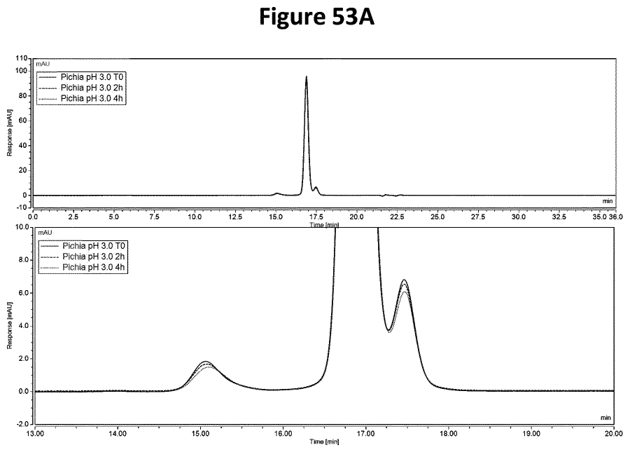

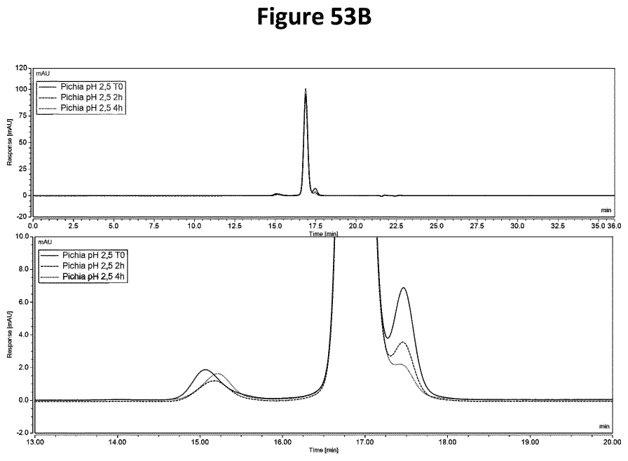

Quality of the compound C protein was evaluated by SE-HPLC. Also for compound C, a distinct post peak was observed in the SE-HPLC (FIGS. 53A and B).

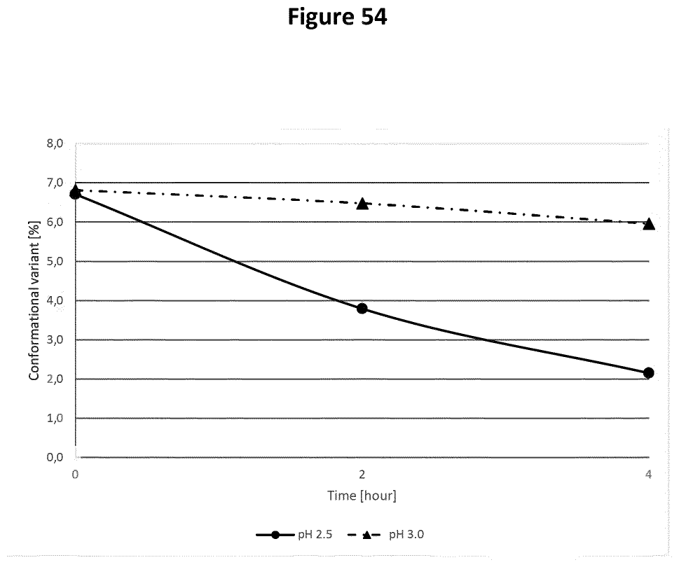

The influence of pH on the product quality was analyzed in function of time by SE-HPLC (see Table 31 and FIG. 54).

TABLE 31
Results of SE-HPLC analysis of the low pH treatment
impact on conversion of the conformational variant.

| pH | Time point [hour] | SE-HPLC Post-peak 1 (%) |
|---|---|---|
| Capture eluate | N/A | 7.0 |
| at pH 3.5 | | |
| Capture eluate | N/A | 6.9 |
| adjusted at pH 5.5 | | |
| 2.5 | 0 | 6.7 |
| 2.5 | 2 | 3.8 |
| 2.5 | 4 | 2.2 |
| 3 | 0 | 6.8 |
| 3 | 2 | 6.5 |
| 3 | 4 | 6.0 |

The SE-HPLC results show the positive influence of a low pH treatment on the presence of conformational variant in the sample. The level of conformational variant in the T0 sample was similar in the two samples tested, i.e., 6.7% of compact variant for the pH 2.5 sample and 6.8% of conformational variant for the pH 3.0 sample. These two values are similar to the initial sample, i.e., the capture eluate not treated by low pH, where the level of conformational variant was 6.9%. After 2 h of incubation at low pH, a decrease of conformational variant was observed for all pHs tested. This decrease was further continued over time until 4 h of incubation at low pH.

All results obtained in this example show the positive impact of low pH on the percentage of conformational variant.

### 6.15 Example 15: Absence of Compact Variant Upon ISVD Production in CHO Cells

After expression of compound C (SEQ ID NO: 69) in CHO cells, a capture chromatography using MabSelect Xtra resin was used to isolate compound C from other impurities.

The column was first equilibrated with Tris buffer and loaded with clarified cell-free harvest material containing the compound of interest. The equilibration buffer contained 50 mM Tris, 150 mM NaCl at pH 7.5. Compound C binds to the MabSelect Xtra resin and impurities flow through the column. Subsequently, the loaded resin was washed with the same Tris buffer as the equilibration step followed by a second wash with Tris wash buffer. The wash buffer contained 10 mM Tris, 10 mM NaCl at pH 7.5. Compound C was eluted from the column with a low pH glycine buffer. The low pH glycine elution buffer contained 50 mM Glycine at pH 3.0. Finally, the resin was regenerated with 100 mM glycine buffer pH 2.5 and cleaned with 50 mM NaOH, 1M NaCl before storage in Et-OH. All buffers were run at 191 cm/h.

After capture chromatography, pH of the product eluting from the chromatography column was 3.4. Compound C was subsequently submitted to low pH incubation. The pH of the capture eluate was decreased to pH 2.5 or pH 3.0 with 1M HCl. After 2 h of incubation at low pH, samples were adjusted to pH 5.5 with 1M HEPES pH 7.0. The capture eluate immediately adjusted to pH 5.5 was the control sample in this experiment.

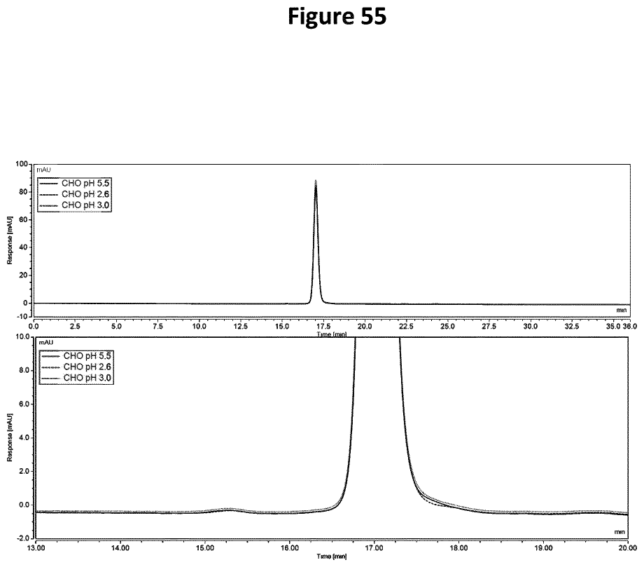

Quality of the compound C protein was evaluated by SE-HPLC. No post peak was observed in the SE-HPLC when compound C was produced in CHO cells (FIG. 55).

The SE-HPLC results showed the absence of conformational variant in the sample.

### 6.16 Example 16: Identification and Initial Characterization of a Conformational Variant of Compound D

Compound D (SEQ ID NO: 70) is a multivalent ISVD construct comprising four immunoglobulin single variable domains of heavy-chain llama antibodies that bind to three different targets. The ISVD building blocks are fused head-to-tail (N-terminus to C-terminus) with a G/S linker in the following format: an TNFα-binding ISVD—9GS linker—IL-6-binding ISVD—9GS linker—a human serum albumin-binding ISVD—9GS linker—a IL-6-binding ISVD and have the following sequence:

TABLE 32
Amino acid sequence of compound D
Compound D (SEQ ID NO: 70)

DVQLVESGGGVVQPGGSLRLSCTASGFTFSTADMGWFRQAPGKGREFVARISGIDGTTYYDEPVKGRF
TISRDNSKNTVYLQMNSLRPEDTALYYCRSPRYADQWSAYDYWGQGTLVTVSSGGGGSGGGSEVQLV
ESGGGVVQPGGSLRLSCAASGRTFSSYVMGWFRQAPGKEREFVSTINWAGSRGYYADSVKGRFTISRD
NAKNTVYLQMNSLRPEDTALYYCAASAGGFLVPRVGQGYDYWGQGTLVTVSSGGGGSGGGSEVQLV
ESGGGVVQPGGSLRLSCAASGFTFRSFGMSWVRQAPGKGPEWVSSISGSGSDTLYADSVKGRFTISRD
NSKNTLYLQMNSLRPEDTALYYCTIGGSLSRSSQGTLVTVSSGGGGSGGGSEVQLVESGGGVVQPGGS
LRLSCAASGFSLDYYGVGWFRQAPGKEREGVSCISSSEGDTYYADSVKGRFTISRDNAKNTVYLQMNSL
RPEDTALYYCATDLSDYGVCSRWPSPYDYWGQGTLVKVSSA

After expression of compound D in Pichia and harvest, a capture chromatography using Amsphere A3 resin was used to isolate compound D from other impurities.

The column was first equilibrated with PBS buffer pH 7.5 and loaded with clarified cell-free harvest material containing the compound of interest. Compound D binds to the Amsphere A3 resin and impurities flow through the column. Subsequently, the loaded resin was washed with the same PBS buffer as the equilibration step. Compound D was eluted from the column with a low pH glycine buffer. The low pH glycine elution buffer contained 100 mM Glycine at pH 3.0. Finally, the resin was cleaned with 100 mM NaOH before storage in the same PBS buffer as equilibration. All buffers were run at 233 cm/h.

Compound D was submitted to low pH incubation. The pH of the capture eluate was decreased to pH 2.5, pH 2.7, pH 2.9, pH 3.1, pH 3.2, pH 3.4 and pH 3.6 with 1M HCl. After a 2 h and 4 h of incubation at low pH, samples were adjusted to pH 5.5 with 0.1 M sodium acetate pH 5.6. The T0 was generated by decreasing compound D to the target low pH (i.e pH 2.3, pH 2.7, pH 2.9, pH 3.1, pH 3.2, pH 3.4 and pH 3.6) with 1M HCl and directly adjusting to pH 5.5 with 1M sodium acetate (T0).

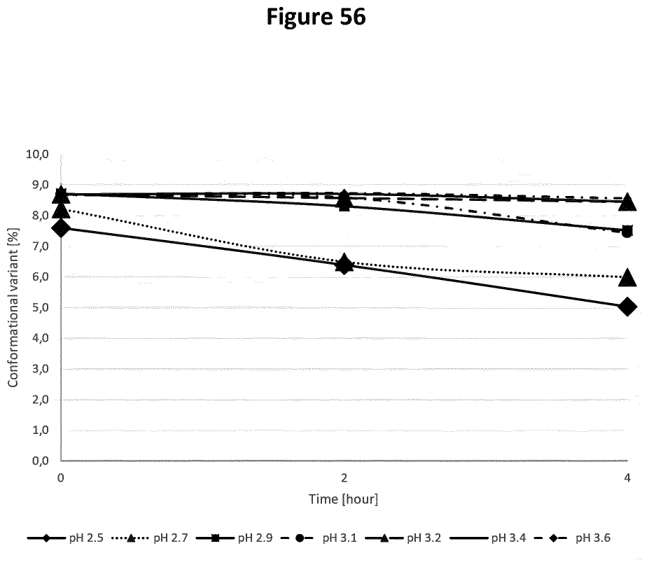

The influence of pH on the product quality in function of time was analyzed by SE-HPLC (Table 33 and FIG. 56).

TABLE 33
Results of SE-HPLC analysis of the low pH treatment impact
on conversion of the conformational variant of compound D.

| pH | Time point [hour] | SE-HPLC Post-peak 1 (%) |
|---|---|---|
| 2.5 | 0 | 7.6 |
| | 2 | 6.4 |
| | 4 | 5.0 |
| 2.7 | 0 | 8.2 |
| | 2 | 6.5 |
| | 4 | 6.0 |
| 2.9 | 0 | 8.7 |
| | 2 | 8.3 |
| | 4 | 7.5 |
| 3.1 | 0 | 8.7 |
| | 2 | 8.6 |
| | 4 | 7.4 |
| 3.2 | 0 | 8.7 |
| | 2 | 8.6 |
| | 4 | 8.5 |
| 3.4 | 0 | 8.7 |
| | 2 | 8.7 |
| | 4 | 8.4 |
| 3.6 | 0 | 8.7 |
| | 2 | 8.7 |
| | 4 | 8.6 |

SE-HPLC results show the positive influence of a low pH treatment on the presence of conformational variants in the sample. The level of conformational variants in the T0 sample was similar in all samples tested. The level of conformational variant in the control samples at T0, pH 2.9, 3.1, 3.2, 3.4 and pH 3.6, was about 8.7%. At lower pH, i.e., pH 2.5, pH 2.7, the start amount was lower (pH 7.6 and pH 8.2) due to the positive influence of the pH.

After 2 h of incubation at low pH, the level of conformational variant decreased. The positive effect of low pH on the conformational variant increased with lower pH.

After 4 h of incubation at low pH, the level of conformational variant further decreased. The best reduction was obtained at pH 2.3 up to pH 3.1.

All results obtained in this example show the positive impact of low pH on conformational variant over time.

### 6.17 Example 17: Identification and Initial Characterization of a Conformational Variant of Compound E

Compound E (SEQ ID NO: 71) is a multivalent ISVD construct comprising four immunoglobulin single variable domains of heavy-chain llama antibodies that bind to three different targets. The ISVD building blocks are fused head-to-tail (N-terminus to C-terminus) with a G/S linker in the following format: an TNFα-binding ISVD—9GS linker—IL-6-binding ISVD—9GS linker—a human serum albumin-binding ISVD—9GS linker—a IL-6-binding ISVD and have the following sequence:

TABLE 34
Amino acid sequence of compound E
Compound E (SEQ ID NO: 71)

DVQLVESGGGVVQPGGSLRLSCTASGFTFSTADMGWFRQAPGKGREFVARISGIDGTTYY
DEPVKGRFTISRDNSKNTVYLQMNSLRPEDTALYYCRSPRYADQWSAYDYWGQGTLVTVS
SGGGGSGGGSEVQLVESGGGVVQPGGSLRLSCAASGIIFSINAMGWYRQAPGKQRELVAD
IFPFGSTEYADSVKGRFTISRDNAKNTVYLQMNSLRPEDTALYYCHSYDPRGDDYWGQGT
LVTVSSGGGGSGGGSEVQLVESGGGVVQPGGSLRLSCAASGFTFRSFGMSWVRQAPGKGP
EWVSSISGSGSDTLYADSVKGRFTISRDNSKNTLYLQMNSLRPEDTALYYCTIGGSLSRS
SQGTLVTVSSGGGGSGGGSEVQLVESGGGVVQPGGSLRLSCAASGRTFSSYVMGWFRQAP
GKEREFVSTINWAGSRGYYADSVKGRFTISRDNAKNTVYLQMNSLRPEDTALYYCAASAG
GFLVPRVGQGYDYWGQGTLVKVSSA

After expression of compound E in Pichia and harvest, a capture chromatography using Amsphere A3 resin was used to isolate compound E from other impurities.

The column was first equilibrated with PBS buffer pH 7.5 and loaded with clarified cell-free harvest material containing the compound of interest. Compound E binds to the Amsphere A3 resin and impurities flow through the column. Subsequently, the loaded resin was washed with the same PBS buffer as the equilibration step. Compound E was eluted from the column with a low pH glycine buffer. The low pH glycine elution buffer contained 100 mM Glycine at pH 3.0. Finally, the resin was cleaned with 100 mM NaOH before storage in the same PBS buffer as equilibration. All buffers were run at 233 cm/h.

Compound E was submitted to low pH incubation. The pH of the capture eluate was decreased to pH 2.5, pH 2.7, pH 2.9, pH 3.1, pH 3.2, pH 3.4 and pH 3.6 with 1M HCl. After a 2 h of incubation at low pH, samples were adjusted to pH 5.5 with 0.1 M sodium acetate pH 5.6. The T0 was generated by decreasing compound E to the target low pH (i.e pH 2.5, pH 2.7, pH 2.9, pH 3.1, pH 3.2, pH 3.4 and pH 3.6) with 1M HCl and directly adjusting to pH 5.5 with 1M sodium acetate (T0).

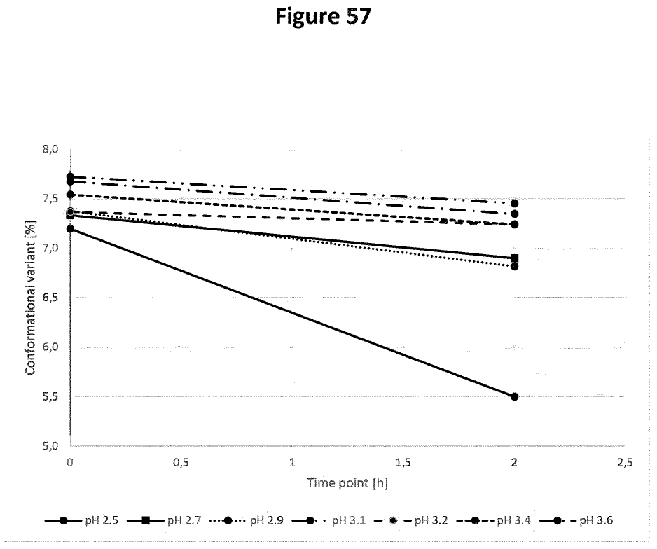

The influence of pH on the product quality in function of time was analyzed by SE-HPLC (Table 35 and FIG. 57).

TABLE 35
Results of SE-HPLC analysis of the low pH treatment impact
on conversion of the conformational variant of compound E.

| pH | Time point [hour] | SE-HPLC Post-peak 1 (%) |
|---|---|---|
| 2.5 | 0 | 7.2 |
| | 2 | 5.5 |
| 2.7 | 0 | 7.3 |
| | 2 | 6.9 |
| 2.9 | 0 | 7.4 |
| | 2 | 6.8 |
| 3.1 | 0 | 7.7 |
| | 2 | 7.5 |
| 3.2 | 0 | 7.4 |
| | 2 | 7.2 |
| 3.4 | 0 | 7.5 |
| | 2 | 7.2 |
| 3.6 | 0 | 7.7 |
| | 2 | 7.3 |

SE-HPLC results show the positive influence of a low pH treatment on the presence of conformational variants in the sample. The level of conformational variants in the T0 sample was similar in all samples tested. The level of conformational variant in the control samples at T0, pH 2.9, 3.1, 3.2, 3.4 and pH 3.6, was about 7.5% (or higher). At lower pH, i.e., pH 2.5, pH 2.7, the start amount was lower (pH 7.2) due to the positive influence of the pH.

After 2 h of incubation at low pH, the level of conformational variant decreased. The positive effect of low pH on the conformational variant increased with lower pH. All results obtained in this example show the positive impact of low pH on conformational variant over time. The best reduction was obtained at pH 2.5 up to pH 2.9.

1. A method of isolating or purifying a polypeptide that comprises or consists of at least three or at least four immunoglobulin single variable domains (ISVDs) from a composition comprising the polypeptide and a conformational variant thereof, the method comprising:
a) applying conditions that convert the conformational variant into the polypeptide;
b) removing the conformational variant; or
c) a combination of (a) and (b).

2. The method according to claim 1, wherein the polypeptide to be isolated or purified is obtainable by expression in a host.

3. The method according to claim 2, wherein the polypeptide to be isolated or purified is obtainable by expression in a host that is not CHO cells.

4. The method according to claim 2 or 3, wherein the polypeptide to be isolated or purified is obtainable by expression in a host that is a lower eukaryotic host.

5. The method according to claim 4, wherein the lower eukaryotic host comprises yeast such as Pichia, Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis.

6. The method according to claim 5, wherein the yeast is Pichia such as Pichia pastoris.

7. The method according to any one of claims 1 to 6, wherein the polypeptide comprises or consists of at least four immunoglobulin single variable domains (ISVDs).

8. The method according to any one of claims 1 to 7, wherein the conformational variant is characterized by a more compact form compared to the polypeptide.

9. The method according to any one of claims 1 to 8, wherein the conformational variant has a decreased hydrodynamic volume compared to the polypeptide.

10. The method according to any one of claims 1 to 9, wherein the conformational variant is characterized by an increased retention time in SE-HPLC compared to the polypeptide.

11. The method according to any one of claims 1 to 10, wherein the conformational variant is characterized by an altered retention time in IEX-HPLC compared to the polypeptide.

12. The method according to any one of claims 1 to 11, wherein the conditions that convert the conformational variant into the polypeptide are selected from:
i) applying a low pH treatment in a step of the isolation or purification process, optionally wherein the low pH treatment comprises decreasing the pH of the composition to about pH 3.2 or less, or to about pH 3.0 or less;
ii) applying a chaotropic agent in a step of the isolation or purification process, optionally wherein the chaotropic agent is guanidinium hydrochloride (GuHCl);
iii) applying a heat stress in a step of the isolation or purification process, optionally comprising incubating the conformational variant at about 40° C. to about 60° C.; or
iv) a combination of any of i) to iii).

13. The method according to any one of claims 1 to 6, wherein the polypeptide comprises or consists of at least four immunoglobulin single variable domains (ISVDs), and wherein the low pH treatment comprises decreasing the pH of the composition to about pH 3.0 or less.

14. The method according to claim 12 or 13, wherein the pH is decreased to between about pH 3.2 and about 2.1, to between about pH 3.0 and about 2.1, to between about pH 2.9 and about pH 2.1, to between about pH 2.7 and about pH 2.1, or to between about pH 2.6 and about pH 2.3.

15. The method according to any one of claims 12 to 14, wherein the low pH treatment is applied for at least about 0.5 hours, for at least about 1 hour, for at least about 2 hours, or for at least about 4 hours.

16. The method according to any one of claims 12 to 15, wherein the pH is decreased to between about pH 3.2 and about pH 2.1 for at least about 0.5 hours, such as for at least about 1.0 hour.

17. The method according to any one of claims 12 to 16, wherein the pH is decreased to between about pH 3.0 and about pH 2.1 for at least about 0.5 hours, such as for at least about 1.0 hour.

18. The method according to any one of claims 12 to 17, wherein the pH is decreased to between about pH 2.9 and about pH 2.1 for at least about 0.5 hours, such as for at least about 1.0 hour.

19. The method according to any one of claims 12 to 18, wherein the pH is decreased to between about pH 2.7 and about pH 2.1 for at least about 0.5 hours, such as for at least about 1.0 hour.

20. The method according to any one of claims 12 to 19, wherein the low pH treatment is applied before a purification step based on a chromatographic technique, during a purification step based on a chromatographic technique or after a purification step based on a chromatographic technique.

21. The method according to claim 20, wherein the low pH treatment is applied before applying the composition to the stationary phase of a chromatographic technique, or after eluting the composition from the stationary phase of a chromatographic technique.

22. The method according to any one of claims 12 to 21, wherein the chaotropic agent is guanidinium hydrochloride (GuHCl) in a final concentration of at least about 1 M, or at least about 2 M.

23. The method according to claims 12 to 22, wherein the GuHCl is applied for at least 0.5 hours, or for at least 1 hour.

24. The method according to any one of claims 12 to 23, wherein the heat stress is applied for at least about 1 hour.

25. The method according to any one of claims 1 to 11, wherein the conformational variant is removed by one or more chromatographic techniques, optionally wherein the conformational variant has been identified by analytical chromatographic techniques such as SE-HPLC and IEX-HPLC before being removed by the one or more chromatographic techniques.

26. The method according to claim 25, wherein the chromatographic technique is a chromatographic technique based on hydrodynamic volume, surface charge or surface hydrophobicity.

27. The method according to claim 26, wherein the chromatographic technique is selected from any of size exclusion chromatography (SEC), ion-exchange chromatography (IEX) e.g., cation-exchange chromatography (CEX), mixed-mode chromatography (MMC), and hydrophobic interaction chromatography (HIC).

28. The method according to claim 27, wherein the HIC is based on a HIC column resin.

29. The method according to claim 27, wherein the HIC is based on a HIC membrane.

30. The method according to any one of claims 1 to 29, wherein isolation or purification of the polypeptide comprises applying the composition to a chromatography column, wherein the composition is applied to the column using a load factor of at least 20 mg protein/ml resin, at least 30 mg protein/ml resin, at least 45 mg protein/ml resin, optionally wherein the chromatographic column is a Protein A column.

31. The method according to any one of claims 1 to 30, wherein one or more of the conditions that convert the conformational variant into the polypeptide are applied alone, or in combination with one or more techniques that remove the conformational variant.

32. A method of isolating or purifying a polypeptide that comprises or consists of at least three or at least four immunoglobulin single variable domains (ISVDs), the method comprising one or more of the following:
i) applying a low pH treatment to a composition comprising the polypeptide in a step of the isolation or purification process, optionally wherein the low pH treatment comprises decreasing the pH of the composition to about pH 3.2 or less, or to about pH 3.0 or less;
ii) applying a chaotropic agent to a composition comprising the polypeptide in a step of the isolation or purification process, optionally wherein the chaotropic agent is GuHCl;
iii) applying a heat stress to a composition comprising the polypeptide in a step of the isolation or purification process, optionally comprising incubating the composition at about 40° C. to about 60° C.;
iv) applying the composition comprising the polypeptide to a chromatography column using a load factor of at least 20 mg/ml, at least 30 mg/ml, at least 45 mg/ml, optionally wherein the chromatographic column is Protein A column; or
v) a combination of any of i) to iv).

33. The method according to claim 32, wherein the polypeptide to be isolated or purified is obtainable by expression in a host.

34. The method according to claim 33, wherein the polypeptide to be isolated or purified is obtainable by expression in a host that is not a CHO cell.

35. The method according to claim 33 or 34, wherein the polypeptide to be isolated or purified is obtainable by expression in a host that is a lower eukaryotic host.

36. The method according to claim 35, wherein the lower eukaryotic host comprises yeast such as Pichia, Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis.

37. The method according to claim 36, wherein the yeast is Pichia such as Pichia pastoris.

38. The method according to any one of claims 32 to 37, wherein the polypeptide comprises or consists of at least four immunoglobulin single variable domains (ISVDs), optionally wherein the low pH treatment comprises decreasing the pH of the composition to about pH 3.0 or less.

39. The method according to any one of claims 32 to 38, wherein the pH is decreased to between about pH 3.2 and about pH 2.1, to between about pH 3.0 and about pH 2.1, to between about pH 2.9 and about pH 2.1, to between about pH 2.7 and about pH 2.1, or to between about pH 2.6 and about pH 2.3.

40. The method according to any one of claims 32 to 39, wherein the low pH treatment is applied for at least about 0.5 hour, for at least about 1 hour, for at least about 2 hours, or for at least about 4 hours.

41. The method according to any one of claim 39 or 40, wherein the pH is decreased to between about pH 3.2 and about pH 2.1 for at least about 0.5 hour, such as for at least about 1.0 hour.

42. The method according to any one of claims 39 to 41, wherein the pH is decreased to between about pH 3.0 and about pH 2.1 for at least about 0.5 hour, such as for at least about 1.0 hour.

43. The method according to any one of claims 39 to 42, wherein the pH is decreased to between about pH 2.9 and about pH 2.1 for at least about 0.5 hour, such as for at least about 1.0 hour.

44. The method according to any one of claims 39 to 43, wherein the pH is decreased to between about pH 2.7 and about pH 2.1 for at least about 0.5 hour, such as for at least about 1.0 hour.

45. The method according to any one of claims 32 to 44, wherein the low pH treatment is applied before a purification step based on a chromatographic technique, during a purification step based on a chromatographic technique or after a purification step based on a chromatographic technique.

46. The method according to claim 45, wherein the low pH treatment is applied before applying the composition to the stationary phase of a chromatographic technique or after eluting the composition from the stationary phase of a chromatographic technique.

47. The method according to any one of claims 32 to 46, wherein the chaotropic agent is GuHCl in a final concentration of at least about 1 M, or at least about 2 M.

48. The method according to any one of claims 32 to 47, wherein the GuHCl is applied for at least 0.5 hours, or for at least 1 hour.

49. The method according to any one of claims 32 to 48, wherein the heat stress is applied for at least about 1 hour.

50. A method of producing a polypeptide that comprises at least three or at least four immunoglobulin single variable domains (ISVDs), wherein the method comprises the purification or isolation according to the method of any one of claims 1 to 49.

51. The method according to claim 50, at least comprising the following steps:
a) Optionally cultivating a host or host cell under conditions that are such that the host or host cell will multiply;
b) maintaining the host or host cell under conditions that are such that the host or host cell expresses and/or produces said polypeptide; and
c) isolating and/or purifying the secreted polypeptide from the medium comprising one or more of the isolation or purification methods according to any of claims 1 to 49.

52. The method according to claim 50 or 51, wherein the host is not a CHO cell.

53. The method according to any one of claims 50 to 52, wherein the host is a lower eukaryotic host.

54. The method according to claim 53, wherein the lower eukaryotic host comprises yeasts such as Pichia, Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis.

55. The method according to claim 54, wherein the yeast is Pichia such as Pichia pastoris.

56. A method for isolating or purifying a polypeptide that comprises or consists of at least three or at least four immunoglobulin single variable domains (ISVDs) from a composition comprising the polypeptide and a conformational variant thereof, the method comprising:
(1) Identifying the conformational variant by analytical chromatographic techniques such as SE-HPLC and IEX-HPLC;
(2) Adjusting the chromatographic conditions to allow specific removal of the conformational variant; and
(3) Removing the conformational variant from the composition comprising the polypeptide and the conformational variant thereof by one or more chromatographic techniques.

57. A method for optimizing one or more chromatographic techniques to allow isolation or purification of a polypeptide that comprises or consists of at least three or at least four immunoglobulin single variable domains (ISVDs) from a composition comprising the polypeptide and a conformational variant thereof by the one or more chromatographic techniques, the method comprising:
(1) Identifying the conformational variant by analytical chromatographic techniques such as SE-HPLC and IEX-HPLC;
(2) Optimizing the chromatographic conditions to allow specific removal of the conformational variant.

58. The method according to claim 56 or 57, wherein the polypeptide to be isolated or purified is obtainable by expression in a host.

59. The method according to claim 58, wherein the polypeptide to be isolated or purified is obtainable by expression in a host that is not a CHO cell.

60. The method according to claim 58 or 59, wherein the polypeptide to be isolated or purified is obtainable by expression in a host that is a lower eukaryotic host.

61. The method according to claim 60, wherein the lower eukaryotic host comprises yeast such as Pichia, Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis

62. The method according to claim 61, wherein the yeast is Pichia such as Pichia pastoris.

63. The method according to claims 56 to 62, wherein the conformational variant is characterized as in claims 8 to 11.

64. The method according to any one of claims 56 to 63, wherein the chromatographic technique is a chromatographic technique based on hydrodynamic volume, surface charge or surface hydrophobicity.

65. The method according to claim 64, wherein the chromatographic technique is selected from any of size exclusion chromatography (SEC), ion-exchange chromatography (IEX), mixed-mode chromatography (MMC), and hydrophobic interaction chromatography (HIC).

66. The method according to claim 65, wherein the ion-exchange chromatography (IEX) is cation-exchange chromatography (CEX).

67. The method according to claim 65, wherein the HIC is based on a HIC column resin.

68. The method according to claim 67 wherein the HIC resin is selected from any of Capto Phenyl ImpRes, Capto Butyl ImpRes, Phenyl HP, and Capto Butyl.

69. The method according to claim 65, wherein the HIC is based on a HIC membrane.


SEQUENCE LISTING (71 sequences)

SEQ ID NO: 1 - protein, 630 aa, Artificial Sequence
  Compound A
DVQLVESGGGVVQPGGSLRLSCAASGRTFSSIYAKGWFRQAPGKEREFVAAISRSGRSTSYADSVKGRFTISRDNSKNTVYLQMNSLRPEDTALYYCAAVGGATTVTASEWDYWGQGTLVTVSSGGGGSGGGSEVQLVESGGGVVQPGGSLRLSCAASGRTFSSIYAKGWFRQAPGKEREFVAAISRSGRSTSYADSVKGRFTISRDNSKNTVYLQMNSLRPEDTALYYCAAVGGATTVTASEWDYWGQGTLVTVSSGGGGSGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVKVSSA

SEQ ID NO: 2 - protein, 517 aa, Artificial Sequence
  Compound B
DVQLVESGGGVVQPGGSLRLSCTASGFTFSTADMGWFRQAPGKGREFVARISGIDGTTYYDEPVKGRFTISRDNSKNTVYLQMNSLRPEDTALYYCRSPRYADQWSAYDYWGQGTLVTVSSGGGGSGGGSEVQLVESGGGVVQPGGSLRLSCAASGRIFSLPASGNIFNLLTIAWYRQAPGKQRELVATIESGSRTNYADSVKGRFTISRDNSKKTVYLQMNSLRPEDTALYYCQTSGSGSPNFWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGVVQPGGSLRLSCAASGRTLSSYAMGWFRQAPGKEREFVARISQGGTAIYYADSVKGRFTISRDNSKNTVYLQMNSLRPEDTALYYCAKDPSPYYRGSAYLLSGSYDSWGQGTLVKVSSA

SEQ ID NO: 3 - protein, 3 aa, Artificial Sequence
  3A linker
AAA

SEQ ID NO: 4 - protein, 5 aa, Artificial Sequence
  5GS linker
GGGGS

SEQ ID NO: 5 - protein, 7 aa, Artificial Sequence
  7GS linker
SGGSGGS

SEQ ID NO: 6 - protein, 8 aa, Artificial Sequence
  8GS linker
GGGGSGGS

SEQ ID NO: 7 - protein, 9 aa, Artificial Sequence
  9GS linker
GGGGSGGGS

SEQ ID NO: 8 - protein, 10 aa, Artificial Sequence
  10GS linker
GGGGSGGGGS

SEQ ID NO: 9 - protein, 15 aa, Artificial Sequence
  15GS linker
GGGGSGGGGSGGGGS

SEQ ID NO: 10 - protein, 18 aa, Artificial Sequence
  18GS linker
GGGGSGGGGSGGGGSGGS

SEQ ID NO: 11 - protein, 20 aa, Artificial Sequence
  20GS linker
GGGGSGGGGSGGGGSGGGGS

SEQ ID NO: 12 - protein, 25 aa, Artificial Sequence
  25GS linker
GGGGSGGGGSGGGGSGGGGSGGGGS

SEQ ID NO: 13 - protein, 30 aa, Artificial Sequence
  30GS linker
GGGGSGGGGSGGGGSGGGGSGGGGSGGGGS

SEQ ID NO: 14 - protein, 35 aa, Artificial Sequence
  35GS linker
GGGGSGGGGSGGGGSGGGGSGGGGSGGGGSGGGGS

SEQ ID NO: 15 - protein, 40 aa, Artificial Sequence
  40GS linker
GGGGSGGGGSGGGGSGGGGSGGGGSGGGGSGGGGSGGGGS

SEQ ID NO: 16 - protein, 15 aa, Artificial Sequence
  G1 hinge
EPKSCDKTHTCPPCP

SEQ ID NO: 17 - protein, 24 aa, Artificial Sequence
  9GS-G1 hinge
GGGGSGGGSEPKSCDKTHTCPPCP

SEQ ID NO: 18 - protein, 12 aa, Artificial Sequence
  Llama upper long hinge region
EPKTPKPQPAAA

SEQ ID NO: 19 - protein, 62 aa, Artificial Sequence
  G3 hinge
ELKTPLGDTTHTCPRCPEPKSCDTPPPCPRCPEPKSCDTPPPCPRCPEPKSCDTPPPCPRCP

SEQ ID NO: 20 - protein, 4 aa, Artificial Sequence
  Motif
KERE

SEQ ID NO: 21 - protein, 4 aa, Artificial Sequence
  Motif
KQRE

SEQ ID NO: 22 - protein, 4 aa, Artificial Sequence
  Motif
GLEW

SEQ ID NO: 23 - protein, 5 aa, Artificial Sequence
  Motif
KEREL

SEQ ID NO: 24 - protein, 5 aa, Artificial Sequence
  Motif
KEREF

SEQ ID NO: 25 - protein, 5 aa, Artificial Sequence
  Motif
KQREL

SEQ ID NO: 26 - protein, 5 aa, Artificial Sequence
  Motif
KQREF

SEQ ID NO: 27 - protein, 5 aa, Artificial Sequence
  Motif
KEREG

SEQ ID NO: 28 - protein, 5 aa, Artificial Sequence
  Motif
KQREW

SEQ ID NO: 29 - protein, 5 aa, Artificial Sequence
  Motif
KQREG

SEQ ID NO: 30 - protein, 4 aa, Artificial Sequence
  Motif
TERE

SEQ ID NO: 31 - protein, 5 aa, Artificial Sequence
  Motif
TEREL

SEQ ID NO: 32 - protein, 4 aa, Artificial Sequence
  Motif
TQRE

SEQ ID NO: 33 - protein, 5 aa, Artificial Sequence
  Motif
TQREL

SEQ ID NO: 34 - protein, 4 aa, Artificial Sequence
  Motif
KECE

SEQ ID NO: 35 - protein, 5 aa, Artificial Sequence
  Motif
KECEL

SEQ ID NO: 36 - protein, 5 aa, Artificial Sequence
  Motif
KECER

SEQ ID NO: 37 - protein, 4 aa, Artificial Sequence
  Motif
KQCE

SEQ ID NO: 38 - protein, 5 aa, Artificial Sequence
  Motif
KQCEL

SEQ ID NO: 39 - protein, 4 aa, Artificial Sequence
  Motif
RERE

SEQ ID NO: 40 - protein, 5 aa, Artificial Sequence
  Motif
REREG

SEQ ID NO: 41 - protein, 4 aa, Artificial Sequence
  Motif
RQRE

SEQ ID NO: 42 - protein, 5 aa, Artificial Sequence
  Motif
RQREL

SEQ ID NO: 43 - protein, 5 aa, Artificial Sequence
  Motif
RQREF

SEQ ID NO: 44 - protein, 5 aa, Artificial Sequence
  Motif
RQREW

SEQ ID NO: 45 - protein, 4 aa, Artificial Sequence
  Motif
QERE

SEQ ID NO: 46 - protein, 5 aa, Artificial Sequence
  Motif
QEREG

SEQ ID NO: 47 - protein, 4 aa, Artificial Sequence
  Motif
QQRE

SEQ ID NO: 48 - protein, 5 aa, Artificial Sequence
  Motif
QQREW

SEQ ID NO: 49 - protein, 5 aa, Artificial Sequence
  Motif
QQREL

SEQ ID NO: 50 - protein, 5 aa, Artificial Sequence
  Motif
QQREF

SEQ ID NO: 51 - protein, 4 aa, Artificial Sequence
  Motif
KGRE

SEQ ID NO: 52 - protein, 5 aa, Artificial Sequence
  Motif
KGREG

SEQ ID NO: 53 - protein, 4 aa, Artificial Sequence
  Motif
KDRE

SEQ ID NO: 54 - protein, 5 aa, Artificial Sequence
  Motif
KDREV

SEQ ID NO: 55 - protein, 5 aa, Artificial Sequence
  Motif
DECKL

SEQ ID NO: 56 - protein, 5 aa, Artificial Sequence
  Motif
NVCEL

SEQ ID NO: 57 - protein, 4 aa, Artificial Sequence
  Motif
GVEW

SEQ ID NO: 58 - protein, 4 aa, Artificial Sequence
  Motif
EPEW

SEQ ID NO: 59 - protein, 4 aa, Artificial Sequence
  Motif
GLER

SEQ ID NO: 60 - protein, 4 aa, Artificial Sequence
  Motif
DQEW

SEQ ID NO: 61 - protein, 4 aa, Artificial Sequence
  Motif
DLEW

SEQ ID NO: 62 - protein, 4 aa, Artificial Sequence
  Motif
GIEW

SEQ ID NO: 63 - protein, 4 aa, Artificial Sequence
  Motif
ELEW

SEQ ID NO: 64 - protein, 4 aa, Artificial Sequence
  Motif
GPEW

SEQ ID NO: 65 - protein, 4 aa, Artificial Sequence
  Motif
EWLP

SEQ ID NO: 66 - protein, 4 aa, Artificial Sequence
  Motif
GPER

SEQ ID NO: 67 - protein, 4 aa, Artificial Sequence
  Motif
GLER

SEQ ID NO: 68 - protein, 4 aa, Artificial Sequence
  Motif
ELEW

SEQ ID NO: 69 - protein, 363 aa, Artificial Sequence
  Compound C
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 70 - protein, 515 aa, Artificial Sequence
  Compound D
DVQLVESGGGVVQPGGSLRLSCTASGFTFSTADMGWFRQAPGKGREFVARISGIDGTTYYDEPVKGRFTISRDNSKNTVYLQMNSLRPEDTALYYCRSPRYADQWSAYDYWGQGTLVTVSSGGGGSGGGSEVQLVESGGGVVQPGGSLRLSCAASGRTFSSYVMGWFRQAPGKEREFVSTINWAGSRGYYADSVKGRFTISRDNAKNTVYLQMNSLRPEDTALYYCAASAGGFLVPRVGQGYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGVVQPGGSLRLSCAASGFSLDYYGVGWFRQAPGKEREGVSCISSSEGDTYYADSVKGRFTISRDNAKNTVYLQMNSLRPEDTALYYCATDLSDYGVCSRWPSPYDYWGQGTLVKVSSA

SEQ ID NO: 71 - protein, 505 aa, Artificial Sequence
  Compound E
DVQLVESGGGVVQPGGSLRLSCTASGFTFSTADMGWFRQAPGKGREFVARISGIDGTTYYDEPVKGRFTISRDNSKNTVYLQMNSLRPEDTALYYCRSPRYADQWSAYDYWGQGTLVTVSSGGGGSGGGSEVQLVESGGGVVQPGGSLRLSCAASGIIFSINAMGWYRQAPGKQRELVADIFPFGSTEYADSVKGRFTISRDNAKNTVYLQMNSLRPEDTALYYCHSYDPRGDDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGVVQPGGSLRLSCAASGRTFSSYVMGWFRQAPGKEREFVSTINWAGSRGYYADSVKGRFTISRDNAKNTVYLQMNSLRPEDTALYYCAASAGGFLVPRVGQGYDYWGQGTLVKVSSA